(19) United States
(12) Patent Application Publication
(10) Pub. No.: US 2024/0312564 A1
(43) Pub. Date: Sep. 19, 2024

(54) WHITE BLOOD CELL CONTAMINATION DETECTION

(71) Applicant: GRAIL, LLC, Menlo Park, CA (US)

(72) Inventors: Sahar Nohzadeh-Malakshah, Union City, CA (US); Qinwen Liu, Fremont, CA (US); Seyedmehdi Shojaee, San Francisco, CA (US); Jing Xiang, San Carlos, CA (US); Joseph Marcus, Burlingame, CA (US); Chenlu Hou, San Carlos, CA (US); John F. Beausang, Menlo Park, CA (US); Jonathan Heiss, Chicago, IL (US); Oliver Claude Venn, San Francisco, CA (US)

(21) Appl. No.: 18/604,046

(22) Filed: Mar. 13, 2024

Related U.S. Application Data
(60) Provisional application No. 63/489,986, filed on Mar. 13, 2023.
(60) Provisional application No. 63/495,766, filed on Apr. 12, 2023.
(60) Provisional application No. 63/518,881, filed on Aug. 11, 2023.

(51) Int. Cl. G16B 30/00; C12Q 1/6869; G16B 40/20

(52) U.S. Cl. CPC G16B 30/00; C12Q 1/6869; G16B 40/20; C12Q 2600/154

(57) ABSTRACT

Methods for WBC contamination detection are disclosed. The computer-implemented methods for WBC contamination detection aim to assess whether a sample is contaminated by the WBC-shed DNA and may further determine a level of contamination. A first coverage-based approach assesses normalized coverage of sequence reads of a test sample at each genomic locus in a feature set of genomic loci. A contamination metric may be calculated based on a distance of the test sample's normalized coverage to a distribution of purified cfDNA samples. A second methylation-based approach deconvolves tissue type based on methylation features. A distribution is generated based on tissue type fractions of purified cfDNA samples from non-cancer subjects. The contamination metric is calculated based on a distance relative to the distribution of tissue type fractions. A third quantitative coverage-based approach generates distributions of coverage for cfDNA samples and for WBC samples for each genomic locus. A contamination metric is calculated as a fractional contribution of WBC-shed DNA that maximizes a likelihood based on the distributions of coverage.

### CROSS-REFERENCE TO RELATED APPLICATIONS

The present application claims the benefit of and priority to U.S. Provisional Application No. 63/489,986 filed on Mar. 13, 2023, U.S. Provisional Application No. 63/495,766 filed on Apr. 12, 2023, and U.S. Provisional Application No. 63/518,881 filed on Aug. 11, 2023, all of which are incorporated by reference.

### BACKGROUND

Cancer is a leading cause of death worldwide. The fatality of cancer is heightened by the fact that cancer is usually detected in latter stages, limiting efficacy of treatment options for long-term survival. Current detection methods generally are cancer type specific, i.e., each cancer type is individually screened for. Each individual screening process is tailored to the cancer type. For example, mammography scans are utilized in breast cancer detection, whereas colonoscopy or fecal tests have helped with colorectal cancer detection. Each varied screening method is not cross-applicable to other cancer types. For example, to screen one individual for three different possible cancer types, a healthcare provider would need to perform or order to be performed three different screening processes. Each of those screening processes may entail a combination of invasive and/or non-invasive procedures to identify tumorous growths, collect a biopsy of the growth, and perform analysis on the tissue biopsy.

Furthermore, present screening methods are encumbered by low detection rates or high false positive rates. Low detection rates often fail to detect early-stage cancers as the cancers are just developing. A high positive rate misdiagnoses cancer-free individuals as positive for cancer status. As a result, most screening tests are only practical when they are used to test individuals who have a high risk of developing the screened cancer, and they have limited ability to detect cancers in the general population.

Novel research has implicated aberrant DNA methylation in many disease processes, including cancer. DNA methylation plays a role in regulating gene expression. Thus, aberrant DNA methylation can create issues in normal gene expression pathways, thereby leading to cancer or other diseases. For example, specific patterns of differentially methylated regions may be useful as molecular markers for various disease states. Nonetheless, even such models face a number of challenges. Early cancer detection is particularly challenging due to the miniscule ratio of tumor cells to non-cancer cells in the subject. The miniscule ratio may be on the order of 1:1000, 1:10,000, or even 1:100,000. This creates a challenge of detecting small amounts of cancer signal amidst healthy signal. Moreover, DNA may be shed by blood cells which may comprise age-related genetic variations, often resembling cancerous aberrant methylation. These informatively methylated fragments shed from blood cells can often ostensibly inflate cancer signal.

Further challenges arise during cfDNA sample preparation with white blood cell (WBC) contamination of plasma in a blood sample. The WBC signal also generally include high genetic variations due to natural operation of the white blood cells. However, this WBC signal is generally predicted to be artificial cancer signal, thereby skewing analyses in cancer classification with the WBC contaminated samples.

The present disclosure is directed to addressing the above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The invention(s) described herein this disclosure provide for improvements to cancer detection and treatment, in particular, improving contamination detection to ensure sample quality is maintained from collection through sequencing and analysis to prediction. The invention(s) describe implementation of one or more contamination prediction model(s). The contamination prediction model performs analyses with sequencing data involving one or more contamination markers to assess WBC contamination in a sample.

Remedial measures may be taken with samples deemed to have been contaminated by WBC-shed cfDNA. As one example, a contaminated sample may be withheld from downstream analyses so as to prevent skewed results. Moreover, contaminated samples may be physically discarded or otherwise labeled unusable. A new sample may also be collected from the individual. Furthermore, the contamination detection workflow may be used to assess the source of contamination. For example, the contamination detection workflow may assess sample quality before and after a step in the sample processing workflow. If there's degradation in the sample quality, then that indicates the tested step to be a contributing factor to the contamination. Or, as another example, the contamination detection workflow may be used to assess sample quality from two parallel workflows (or two sequencing assays) to assess which better maintains sample quality. This improved contamination detection improves the assaying of samples to ensure accuracy of analytical predictions and to avoid returning predictions based on contaminated samples.

The invention(s) comprise screening for cancer signal in a cell-free deoxyribonucleic acid (cfDNA) sample of a subject. Such cfDNA samples may comprise thousands, tens of thousands, hundreds of thousands, millions, or more of cfDNA fragments, thereby resulting in a similar order of sequence reads output by a sequencer, or even a multiple of such order based on a sequencing depth of the sample. Each sequence read relating to cfDNA fragments can vary in length, e.g., up to 250, 300, 350, 400, 450, 500, 550, 600, 650, 700, 750, 800, 850, 900, 950, or 1000 bp in length. These next-generation sequencing techniques greatly increase volume of fragments that can be sequenced and analyzed, thereby enabling such models to identify even miniscule amounts of cancer signal in a sample. The invention(s) are capable of screening for cancer generally, or for a plurality of cancer types from a single sample. This improves over conventional screening methods tailored per cancer type by providing a single comprehensive screening that is capable of screening a variety of cancer types from a single cfDNA sample. For example, screening for different cancers generally involved separate screening processes that would target detecting tissue growths

The invention(s) implement computer models to assess whether a sample is contaminated by WBC-shed DNA. This process may be termed WBC contamination detection. Generally, blood samples used in cancer detection is processed to separate out the plasma, a buffy coat containing WBC, and solid red blood cells and other particulates. The plasma generally contains cfDNA fragments that are shed from cells in the body. In some patients with cancer, the tumorous cells shed cfDNA fragments into the plasma. As such, cancer signal can be detected by analysis of the cfDNA. However, white blood cells are also prone to shed DNA fragments through their normal course of operation. Such DNA shed by the white blood cells may confound cancer detection when analyzed in conjunction with the cfDNA in the plasma, which may lead to false positive calls of cancer. The computer models implemented in WBC contamination detection aim to assess whether a sample is contaminated by the WBC-shed DNA and may further determine a level of contamination. Such screening ensures training data used to train downstream models will not be skewed due to the WBC contamination. The screening also can reduce false positive predictions by withholding contaminated samples from further analyses. Upon detection of a contaminated sample, further remedial measures may be taken to address the contamination. Remedying sources of contamination thereby improves the physical assaying process and the sample processing.

The invention(s) implement computer models to identify and quantify the cancer signal. In one or more embodiments, the computer models may identify informatively methylated fragments (also referred to fragments with informative methylation patterns). From the informatively methylated fragments, computer models may be trained to identify features for use in cancer classification. The computers models may include trained cancer classifiers configured to input a feature vector generated based on the informatively methylated fragments and to output a cancer prediction based on the input feature vector. The cancer prediction may be a binary prediction and/or a multiclass prediction. The binary prediction may be a likelihood of presence of cancer. The multiclass prediction may be a likelihood of a particular cancer type from a plurality of cancer types evaluated. Training a cancer classifier capable of screening between a plurality of cancer types enables medical care professionals to utilize a single comprehensive screening rather than multiple disparate screenings. In one or more embodiments, the cancer classifier is a machine-learning model rooted in computer functionality and not practically performable in the human mind. The cancer prediction can be used by a healthcare provider for diagnosis, prognosis, tailoring treatment, evaluation of treatment, minimum residual disease detection, etc.

Clause 1. A method for identifying contamination markers for detecting white blood cell (WBC) contamination in a test sample comprising sequence reads corresponding to cell-free DNA (cfDNA) fragments, the method comprising: obtaining a first set of cfDNA samples from a first cohort of subjects and a second set of WBC samples from the first cohort of subjects determining, for each sample, a coverage of sequence reads overlapping each genomic locus in an initial set of genomic loci; determining, for each genomic locus, a ratio of coverage between the WBC samples and the cfDNA samples; and determining a feature set of genomic loci as contamination markers with ratios of coverage above a threshold.

Clause 2. The method of clause 1 or any other clause dependent thereon, wherein each cfDNA sample consists essentially of sequence reads corresponding to cfDNA fragments, and wherein each WBC sample consists essentially of sequence reads corresponding to DNA fragments shed from WBC.

Clause 3. The method of clause 1 or any other clause dependent thereon, wherein each subject of the first cohort is associated with at least one cfDNA sample and at least one WBC sample.

Clause 4. The method of clause 1 or any other clause dependent thereon, further comprising: normalizing, for each sample, the coverage of sequence reads overlapping each genomic locus based on a sequencing depth of the sample.

Clause 5. The method of clause 1 or any other clause dependent thereon, wherein each genomic locus includes at least one CpG site.

Clause 6. The method of clause 1 or any other clause dependent thereon, further comprising: pruning one or more genomic loci with highly variable coverage across samples to generate the initial set of genomic loci.

Clause 7. The method of clause 1 or any other clause dependent thereon, further comprising: pruning one or more genomic loci with coverages that are non-normally distributed across samples.

Clause 8. The method of clause 3, wherein determining, for each genomic locus, the ratio of coverage between the WBC samples and the cfDNA samples comprises: calculating, for each subject in the first cohort, a ratio of coverage for each genomic locus by dividing the coverage at the genomic locus from the WBC sample by the coverage at the genomic locus from the cfDNA sample; and determining the ratio of coverage for each genomic locus as an average of the ratios of coverage at the genomic locus across the subjects in the first cohort.

Clause 9. A method for training a contamination model for detecting white blood cell (WBC) contamination in a test sample comprising sequence reads corresponding to cell-free DNA (cfDNA) fragments, the method comprising: obtaining a set of cfDNA samples from non-cancer subjects, each cfDNA sample comprising sequence reads corresponding to cfDNA fragments; determining, for each sample, a coverage of sequence reads overlapping each genomic locus in a feature set of genomic loci as contamination markers; generating, for each genomic locus in the feature set of genomic loci, a distribution of coverage based on coverage of a first subset of cfDNA samples; determining, for each cfDNA sample of a second subset, a statistical likelihood of observing the coverage at each genomic locus based on the distribution of coverage for the genomic locus; generating, for each cfDNA sample of the second subset, a contamination metric by combining the statistical likelihoods of the cfDNA sample across the genomic loci; determining a distribution of contamination metric for the second subset of cfDNA samples; and determining a contamination threshold based on the distribution of contamination metric, wherein the contamination model comprises the distributions of coverage across the feature set of genomic loci, the distribution of contamination metric, and the contamination threshold.

Clause 10. The method of clause 9 or any other clause dependent thereon, further comprising: normalizing, for each sample, the coverage of sequence reads overlapping each genomic locus based on a sequencing depth of the sample.

Clause 11. The method of clause 9 or any other clause dependent thereon, wherein each genomic locus includes at least one CpG site.

Clause 12. The method of clause 9 or any other clause dependent thereon, wherein the feature set of genomic loci is identified according to the method of clause 1 or any other clause dependent thereon.

Clause 13. The method of clause 9 or any other clause dependent thereon, wherein the statistical likelihood of observing the coverage at each genomic locus is a z-score based on a mean and a standard deviation defining the distribution for the genomic locus.

Clause 14. The method of clause 13, wherein the contamination metric combines absolute values of the z-scores.

Clause 15. The method of clause 9 or any other clause dependent thereon, wherein the statistical likelihood of observing the coverage at each genomic locus is a p-value based on a mean and a standard deviation defining the distribution for the genomic locus.

Clause 16. The method of clause 15, wherein the contamination metric is a truncated p-value product of the p-values of the cfDNA sample across the genomic loci.

Clause 17. The method of clause 9 or any other clause dependent thereon, wherein the contamination threshold is determined to achieve a set specificity for the contamination model.

Clause 18. The method of clause 17, wherein the specificity is one of: 95%, 96%, 97%, 98%, 99%, and 99.5%.

Clause 19. A method for detecting white blood cell (WBC) contamination in a test sample comprising sequence reads corresponding to cell-free DNA (cfDNA) fragments, the method comprising: determining, for each of a feature set of genomic loci as contamination markers, a coverage based on a count of the sequence reads of the cfDNA fragments that overlap a genomic locus; determining, for each of the feature set of genomic loci, a statistical likelihood of observing the coverage at the genomic locus based on a distribution of coverage for the genomic locus generated from purified cfDNA samples; generating a contamination metric for the test sample by combining the statistical likelihoods across the plurality of contamination markers; determining whether the test sample has WBC contamination if the contamination metric for the test sample crosses a contamination threshold; and in response to determining that the test sample has WBC contamination, performing one or more remedial measures.

Clause 20. The method of clause 19 or any other clause dependent thereon, wherein the feature set of genomic loci is identified according to the method of clause 1 or any other clause dependent thereon.

Clause 21. The method of clause 19 or any other clause dependent thereon, wherein the distributions of coverage for the genomic loci are generated according to the method of clause 9 or any other clause dependent thereon.

Clause 22. The method of clause 19 or any other clause dependent thereon, wherein the contamination threshold is identified according to the method of clause 9 or any other clause dependent thereon.

Clause 23. The method of clause 19 or any other clause dependent thereon, wherein the purified cfDNA samples consist essentially of sequence reads corresponding to cfDNA fragments.

Clause 24. The method of clause 19 or any other clause dependent thereon, wherein the statistical likelihood of observing the coverage at each genomic locus is a z-score based on a mean and a standard deviation defining the distribution for the genomic locus.

Clause 25. The method of clause 24 or any other clause dependent thereon, wherein the contamination metric combines absolute values of the z-scores.

Clause 26. The method of clause 24 or any other clause dependent thereon, wherein determining whether the contamination metric for the test sample crosses the contamination threshold comprises determining whether the contamination metric is above the contamination threshold.

Clause 27. The method of clause 19 or any other clause dependent thereon, wherein the statistical likelihood of observing the coverage at each genomic locus is a p-value based on a mean and a standard deviation defining the distribution for the genomic locus.

Clause 28. The method of clause 27 or any other clause dependent thereon, wherein the contamination metric is a truncated p-value product of the p-values of the cfDNA sample across the genomic loci.

Clause 29. The method of clause 27 or any other clause dependent thereon, wherein determining whether the contamination metric for the test sample crosses the contamination threshold comprises determining whether the contamination metric is below the contamination threshold.

Clause 30. The method of clause 19 or any other clause dependent thereon, wherein the remedial measures include any combination of: providing a notification to a healthcare provider that the test sample has WBC contamination; discarding the test sample; labeling the test sample as contaminated; providing a notification to a healthcare provider to collect a subsequent test sample; providing a notification to a clinician of a likely source of WBC contamination; and withholding the test sample from downstream analyses, optionally including cancer classification.

Clause 31. A method for training a deconvolution model configured to deconvolve tissue type fractional contribution in a cell-free DNA (cfDNA) sample, the method comprising: obtaining sets of cfDNA samples from different tissue types, wherein each cfDNA sample comprises methylation sequence reads corresponding to cfDNA fragments; determining, for each cfDNA sample, a methylation feature at each genomic locus in an initial set of genomic loci based on the methylation sequence reads; generating, for each sample, a feature vector based on the methylation features over the initial set of genomic loci; and training a deconvolution model to predict tissue type fractions based on the feature vectors from the samples.

Clause 32. The method of clause 31 or any other clause dependent thereon, further comprising: identifying a feature set of genomic loci based on information gain of the methylation feature of each genomic locus based on the trained deconvolution model; and retraining the deconvolution model with updated feature vectors based on the feature set of genomic loci.

Clause 33. The method of clause 31 or any other clause dependent thereon, wherein the tissue types consist of the group of: erythrocyte progenitor cell type, megakaryocyte cell type, monocyte cell type, granulocyte cell type, lymphocyte cell type, epithelial cell type, vascular endothelial cell type, hepatocyte cell type, adipocyte cell type, endocrine cell type, and muscle cell type.

Clause 34. The method of clause 31 or any other clause dependent thereon, wherein the tissue types consist of the group of: myeloid circulating tissue type, myeloid non-circulating tissue type, lymphocyte cell type, epithelial cell type, vascular endothelial cell type, and hepatocyte cell type.

Clause 35. The method of clause 31 or any other clause dependent thereon, wherein each cfDNA sample is purified.

Clause 36. The method of clause 31 or any other clause dependent thereon, wherein each genomic locus covers at least one CpG site.

Clause 37. The method of clause 31 or any other clause dependent thereon, wherein the methylation feature at each genomic locus is one of: methylation density across methylation sequence reads of the cfDNA sample at the genomic locus; a count or a proportion of methylation sequence reads of the cfDNA sample that are highly methylated and overlap the genomic locus; a count or a proportion of methylation sequence reads of the cfDNA sample that are highly unmethylated and overlap the genomic locus; and a count or a proportion of methylation sequence reads having a particular methylation variant at the genomic locus.

Clause 38. The method of clause 31 or any other clause dependent thereon, wherein the deconvolution model is a machine-learning model.

Clause 39. The method of clause 31 or any other clause dependent thereon, wherein the trained deconvolution model is configured to output a prediction of tissue type fractions, wherein each tissue type fraction is a fractional contribution of a tissue type to the cfDNA fragments of a cfDNA sample.

Clause 40. The method of clause 31 or any other clause dependent thereon, wherein the information gain indicates discriminative power of the methylation feature of each genomic locus in discriminating between two tissue types.

Clause 41. A method for training a contamination model for detecting white blood cell (WBC) contamination in a test sample comprising sequence reads corresponding to cell-free DNA (cfDNA) fragments, the method comprising: obtaining a set of cfDNA samples from non-cancer subjects, wherein each cfDNA sample comprises methylation sequence reads corresponding to cfDNA fragments; determining, for each cfDNA sample, a methylation feature at each genomic locus in a feature set of genomic loci based on the methylation sequence reads; generating, for each cfDNA sample, a feature vector based on the methylation features over the feature set of genomic loci; applying, to the feature vector of each cfDNA sample, a trained deconvolution model to predict tissue type fractions for the cfDNA sample; and building a distribution of the tissue type fractions for the cfDNA samples, wherein the contamination model comprises the distribution.

Clause 42. The method of clause 41 or any other clause dependent thereon, wherein the feature set of genomic loci is identified and/or the deconvolution model is trained according to the method of clause 31 or any other clause dependent thereon.

Clause 43. The method of clause 41 or any other clause dependent thereon, wherein each cfDNA sample is purified.

Clause 44. The method of clause 41 or any other clause dependent thereon, wherein each genomic locus covers at least one CpG site.

Clause 45. The method of clause 41 or any other clause dependent thereon, wherein the methylation feature at each genomic locus is one of: methylation density across methylation sequence reads of the cfDNA sample at the genomic locus; a count or a proportion of methylation sequence reads of the cfDNA sample that are highly methylated and overlap the genomic locus; a count or a proportion of methylation sequence reads of the cfDNA sample that are highly unmethylated and overlap the genomic locus; and a count or a proportion of methylation sequence reads having a particular methylation variant at the genomic locus.

Clause 46. The method of clause 41 or any other clause dependent thereon, wherein the distribution is a multivariate distribution in multivariate vector space with the number of variables equal to the number of tissue types.

Clause 47. A method for detecting white blood cell (WBC) contamination in a test sample comprising methylation sequence reads corresponding to cell-free DNA (cfDNA) fragments, the method comprising: determining a methylation feature at each genomic locus in a feature set of genomic loci based on the methylation sequence reads; generating a feature vector based on the methylation features over the feature set of genomic loci; applying, to the feature vector of the test sample, a trained deconvolution model to predict tissue type fractions for the test sample; generating a contamination metric for the test sample based on a distance of the tissue type fractions for the test sample relative to a distribution of tissue type fractions generated from cfDNA samples from non-cancer subjects, wherein the contamination metric indicates a likelihood that the cfDNA sample has WBC contamination; determining whether the test sample has WBC contamination if the contamination metric for the test sample crosses a contamination threshold; and in response to determining that the test sample has WBC contamination, performing one or more remedial measures.

Clause 48. The method of clause 47 or any other clause dependent thereon, wherein the feature set of genomic loci is identified and/or the deconvolution model is trained according to the method of clause 31 or any other clause dependent thereon.

Clause 49. The method of clause 47 or any other clause dependent thereon, wherein the deconvolution model is trained according to the method of clause 41 or any other clause dependent thereon.

Clause 50. The method of clause 47 or any other clause dependent thereon, wherein each genomic locus covers at least one CpG site.

Clause 51. The method of clause 47 or any other clause dependent thereon, wherein the methylation feature at each genomic locus is one of: methylation density across methylation sequence reads of the cfDNA sample at the genomic locus; a count or a proportion of methylation sequence reads of the cfDNA sample that are highly methylated and overlap the genomic locus; a count or a proportion of methylation sequence reads of the cfDNA sample that are highly unmethylated and overlap the genomic locus; and a count or a proportion of methylation sequence reads having a particular methylation variant at the genomic locus.

Clause 52. The method of clause 47 or any other clause dependent thereon, wherein the distance is a Mahalanobis distance based on the distribution of tissue type fractions.

Clause 53. The method of clause 47 or any other clause dependent thereon, wherein the contamination metric is a p-value.

Clause 54. The method of clause 53, wherein determining whether the contamination metric for the test sample crosses the contamination threshold comprises determining whether the contamination metric is below the contamination threshold.

Clause 55. The method of clause 47 or any other clause dependent thereon, wherein the contamination threshold is determined to achieve a set specificity for the contamination model.

Clause 56. The method of clause 47 or any other clause dependent thereon, wherein the remedial measures include any combination of: providing a notification to a healthcare provider that the test sample has WBC contamination; discarding the test sample; labeling the test sample as contaminated; providing a notification to a healthcare provider to collect a subsequent test sample; providing a notification to a clinician of a likely source of WBC contamination; and withholding the test sample from downstream analyses optionally including cancer classification.

Clause 57. A method for training a contamination model for detecting white blood cell (WBC) contamination in a test sample comprising sequence reads corresponding to cell-free DNA (cfDNA) fragments, the method comprising: obtaining a first set of cfDNA samples and a second set of WBC samples, each sample comprising sequence reads corresponding to DNA fragments; determining, for each sample of the first set and the second set, a mean coverage of sequence reads overlapping an initial set of genomic loci; determining, for each sample of the first set and the second set, a normalized coverage for each genomic locus in the initial set of genomic loci based on sequence reads overlapping the genomic locus normalized by the mean coverage of the sample; and generating, for each genomic locus, a first distribution of coverage for cfDNA samples and a second distribution of coverage for WBC samples; identifying highly discriminatory genomic loci between cfDNA samples and WBC samples based on the distributions of coverage; determining a discriminatory score for each genomic locus with a two-sample t-test; and determining a feature set of genomic loci as contamination markers based on the discriminatory scores.

Clause 58. The method of clause 57 or any other clause dependent thereon, wherein each cfDNA sample consists essentially of sequence reads corresponding to cfDNA fragments, and wherein each WBC sample consists essentially of sequence reads corresponding to DNA fragments shed from WBC.

Clause 59. The method of clause 57 or any other clause dependent thereon, wherein each genomic locus includes at least one CpG site.

Clause 60. The method of clause 57 or any other clause dependent thereon, further comprising: pruning one or more genomic loci with highly variable coverage across samples to generate the initial set of genomic loci.

Clause 61. The method of clause 57 or any other clause dependent thereon, further comprising: pruning one or more genomic loci with coverages that are non-normally distributed across samples.

Clause 62. The method of clause 57 or any other clause dependent thereon, wherein identifying highly discriminatory genomic loci between cfDNA samples and WBC samples based on the distributions of coverage comprises: assessing, for each genomic locus, a distance between the first distribution of coverage for cfDNA samples and the second distribution of coverage for WBC samples.

Clause 63. The method of clause 62, wherein the distance for each genomic locus is based on an area under the curve (AUC) for binary classification between cfDNA and WBC based on coverage at the genomic locus.

Clause 64. The method of clause 63, wherein the distance for each genomic locus is further based on a Hodges-Lehmann estimator.

Clause 65. The method of clause 57 or any other clause dependent thereon, wherein the two-sample t-test evaluates the distinctiveness of paired cfDNA sample and WBC sample for each subject.

Clause 66. The method of clause 57 or any other clause dependent thereon, wherein the discriminatory score is based on Bonferroni correction.

Clause 67. The method of clause 57 or any other clause dependent thereon, further comprising: pruning any genomic locus with non-normal distribution of coverage for cfDNA samples or non-normal distribution of coverage for WBC samples.

Clause 68. The method of clause 57 or any other clause dependent thereon, further comprising: pruning any genomic locus with a difference between a mean of the distribution of coverage for cfDNA samples and a mean of the distribution of coverage for WBC samples below a threshold.

Clause 69. The method of clause 57 or any other clause dependent thereon, wherein determining the feature set of genomic loci as contamination markers based on the discriminatory scores comprises: identifying genomic loci with a discriminatory score above a threshold.

Clause 70. The method of clause 57 or any other clause dependent thereon, wherein determining the feature set of genomic loci as contamination markers based on the discriminatory scores comprises: ranking the initial set of genomic loci based on the discriminatory scores; and selecting a top number of genomic loci from the ranking to form the feature set of genomic loci.

Clause 71. A method for detecting white blood cell (WBC) contamination in a test sample comprising sequence reads corresponding to cell-free DNA (cfDNA) fragments, the method comprising: determining a mean coverage of sequence reads overlapping a feature set of genomic loci; determining a normalized coverage for each genomic locus in the feature set of genomic loci based on sequence reads overlapping the genomic locus normalized by the mean coverage of the sample; applying a contamination model to determine a contamination metric as a fractional contribution of WBC-shed DNA to the test sample that maximizes a likelihood of observing the normalized coverages over the feature set based on distributions of coverage for cfDNA samples and for WBC samples for the feature set of genomic loci; determining whether the test sample has WBC contamination if the contamination metric for the test sample crosses a contamination threshold; and in response to determining that the test sample has WBC contamination, performing one or more remedial measures.

Clause 72. The method of clause 71 or any other clause dependent thereon, wherein the feature set of genomic loci is identified, the contamination model is trained, and/or the distributions of coverage for cfDNA samples and for WBC samples are generated according to the method of clause 57 or any other clause dependent thereon.

Clause 73. The method of clause 71 or any other clause dependent thereon, wherein the contamination model comprises, for each genomic locus, a first distribution of coverage for cfDNA samples and a second distribution of coverage for WBC samples.

Clause 74. The method of clause 71 or any other clause dependent thereon, wherein determining whether the contamination metric for the test sample crosses the contamination threshold comprises determining whether the contamination metric is below the contamination threshold.

Clause 75. The method of clause 71 or any other clause dependent thereon, wherein the contamination threshold is determined to achieve a set specificity for the contamination model.

Clause 76. The method of clause 71 or any other clause dependent thereon, wherein the remedial measures include any combination of: providing a notification to a healthcare provider that the test sample has WBC contamination; discarding the test sample; labeling the test sample as contaminated; providing a notification to a healthcare provider to collect a subsequent test sample; providing a notification to a clinician of a likely source of WBC contamination; and withholding the test sample from downstream analyses optionally including cancer classification.

Clause 77. A method for determining a source of white blood cell (WBC) contamination in one or more samples comprising sequence reads corresponding to cell-free DNA (cfDNA) fragments, the method comprising: obtaining a first set of samples from a first sample processing workflow; obtaining a second set of samples from a second sample processing workflow, wherein the second sample processing workflow includes a first protocol different from the first sample processing workflow, a first clinical product different from the first sample processing workflow, a first sequencing device, or some combination thereof; applying a contamination model to the first set of samples and the second set of samples to determine a contamination metric for each sample; determining a first aggregate metric for the first sample processing workflow based on a combination of the contamination metrics for the first set of samples; determining a second aggregate metric for the second sample processing workflow based on a combination of the contamination metrics for the second set of samples; determining that the source of the WBC contamination arises from the first protocol, the first clinical product, the first sequencing device, or some combination thereof based on determining that the second aggregate metric is greater than the first aggregate metric; and performing one or more remedial measures to mitigate the WBC contamination from the second sample processing workflow.

Clause 78. The method of clause 77, wherein the contamination model is trained according to the method of: clause 9 or any other clause dependent thereon; clause 41 or any other clause dependent thereon; or clause 57 or any other clause dependent thereon.

Clause 79. The method of clause 77 or any other clause dependent thereon, wherein the remedial measures include any combination of: changing the first protocol in the second sample processing workflow to another protocol; changing the first clinical product in the second sample processing workflow to another clinical product; changing the first sequencing device in the second sample processing workflow to another sequencing device; obtaining a third set of samples in place of the second set of samples sequenced from the first sample processing workflow; and discarding the second set of samples obtained from the second sample processing workflow.

Clause 80. The method of clause 77 or any other clause dependent thereon, wherein the first aggregate contamination metric is an average of the contamination metrics of the first set of samples, and wherein the second aggregate contamination metric is an average of the contamination metrics of the second set of samples.

Clause 81. The method of clause 77 or any other clause dependent thereon, wherein the first aggregate contamination metric is a first percentage or a first count of samples in the first set of samples having a corresponding contamination metric above a contamination threshold, and wherein the second aggregate contamination metric is a second percentage or a second count of samples in the second set of samples having a corresponding contamination metric above the contamination threshold.

Clause 82. A method for training a cancer classifier with samples comprising sequence reads corresponding to cell-free DNA (cfDNA) fragments, the method comprising: obtaining a first set of samples obtained from a first cohort of healthy subjects and a second set of samples obtained from a second cohort of subjects diagnosed with cancer; applying a contamination model to the first set of samples and the second set of samples to determine a contamination metric for each sample indicating an amount of white blood cell (WBC) contamination in the sample; determining one or more of the samples to be contaminated having a corresponding contamination metric above a contamination threshold; filtering the contaminated samples, optionally wherein filtering comprises discard the contaminated samples; determining a feature vector for each remaining sample based on the sequence reads of that sample; and training the cancer classifier with the feature vectors for the remaining samples, wherein the trained cancer classifier is configured to predict likelihood of presence of cancer based on an input feature vector derived based on sequence reads in a test cfDNA sample.

Clause 83. The method of clause 82, wherein the contamination model is trained according to the method of: clause 9 or any other clause dependent thereon; clause 41 or any other clause dependent thereon; or clause 57 or any other clause dependent thereon.

Clause 84. The method of clause 82 or any clause dependent thereon, wherein the cancer classifier is a machine-learning model.

Clause 85. The method of clause 82 or any clause dependent thereon, wherein the cancer classifier is trained to predict a binary prediction between presence of cancer or absence of cancer.

Clause 86. The method of clause 82 or any clause dependent thereon, wherein the second set of samples include one or more subsets of samples, wherein each subset of samples are obtained from subjects diagnosed from one of a plurality of cancer types.

Clause 87. The method of clause 86, wherein the cancer classifier is trained to predict a multiclass prediction as a likelihood of presence of one of the plurality of cancer types.

Clause 88. The method of clause 82 or any clause dependent thereon, wherein the sequence reads for each sample are methylation sequence reads, and wherein the feature vector is based on methylation sequence reads with an informative methylation pattern.

Clause 89. A non-transitory computer-readable storage medium storing instructions that, when executed by one or more computer processors, cause the one or more computer processors to perform the method of any of the preceding clauses.

Clause 90. A system comprising: one or more computer processors; and the non-transitory computer-readable storage medium of clause 89.

Clause 91. A treatment kit comprising: a collection vessel for collecting a DNA sample from a subject; optionally, one or more reagents for isolating DNA fragments in the DNA sample; optionally, one or more contamination probes targeting one or more genomic loci selected from Table 1; and the non-transitory computer-readable storage medium of clause 89.

### BRIEF DESCRIPTION OF DRAWINGS

FIG. 1 is an exemplary flowchart describing an overall workflow of cancer classification of a sample, according to one or more embodiments.

FIG. 2A is an exemplary flowchart describing a process of sequencing a fragment of cell-free (cf) DNA to obtain a methylation state vector, according to one or more embodiments.

FIG. 2B is an exemplary illustration of the process of FIG. 2A of sequencing a fragment of cell-free (cf) DNA to obtain a methylation state vector, according to one or more embodiments.

FIG. 3A illustrates a flowchart describing the identification of features for Coverage-Based WBC Contamination Detection, according to one or more embodiments.

FIG. 3B illustrates a flowchart describing the training of a contamination model to predict WBC contamination based on coverage over a feature set of genomic loci, according to one or more embodiments.

FIG. 3C illustrates a flowchart describing prediction of WBC contamination with the contamination model, according to one or more embodiments.

FIG. 4A illustrates a flowchart describing the training of a deconvolution model for Methylation-Based WBC Contamination Detection, according to one or more embodiments.

FIG. 4B illustrates a flowchart describing the training of a contamination model to predict WBC contamination based on predicted tissue type fractions, according to one or more embodiments.

FIG. 4C illustrates a flowchart describing WBC contamination prediction with the contamination model, according to one or more embodiments.

FIG. 5A illustrates a flowchart describing the feature identification and training of a contamination model for Quantitative Coverage-Based WBC Contamination Detection, according to one or more embodiments.

FIG. 5B illustrates a flowchart describing WBC contamination prediction with the contamination model, according to one or more embodiments.

FIG. 6A is an exemplary flowchart describing a process of generating a control group data structure for determining informatively methylated fragments, according to one or more embodiments.

FIG. 6B is an exemplary flowchart describing a process of determining a fragment to be informatively methylated based on the control group data structure, according to one or more embodiments.

FIG. 7A is an exemplary flowchart describing a process of training a cancer classifier, according to one or more embodiments.

FIG. 7B illustrates an example generation of feature vectors used for training the cancer classifier, according to one or more embodiments.

FIG. 8A illustrates an exemplary flowchart of devices for sequencing nucleic acid samples according to one or more embodiments.

FIG. 8B is an exemplary block diagram of an analytics system, according to one or more embodiments.

FIG. 9A illustrates a volcano plot of ratios of coverage for an initial set of genomic loci considered, according to example implementations.

FIG. 9B illustrates a visual comparison of differential coverage at the selected genomic loci between cfDNA samples and WBC samples, according to example implementations.

FIG. 10 illustrates the one genomic locus in mitochondrial DNA with stark difference in representation between cfDNA samples and WBC samples, according to example implementations.

FIG. 11 illustrates a comparison of significant genomic loci in a feature set of genomic loci compared to randomly selected candidate sets, according to example implementations.

FIG. 12 illustrates example paired WBC samples and cfDNA samples from the same subjects assessed over the feature set of genomic loci, according to example implementations.

FIG. 13 illustrates samples identified as WBC contaminated by the coverage-based WBC contamination detection, according to example implementations.

FIG. 14 illustrates samples with longest fragments (top 1%) identified as WBC contaminated by the coverage-based WBC contamination detection, according to example implementations.

FIG. 15 illustrates fractional contributions of various tissue types for three different sample types: cfDNA samples, purified plasma samples, and serum samples containing only WBC DNA, according to example implementations.

FIG. 16 illustrates information gain across a set of genomic loci evaluated for inclusion as part of the deconvolution model, according to example implementations.

FIG. 17 illustrates box plots showing the predictive power of the methylation-based WBC contamination detection methodology, according to example implementations.

FIG. 18 illustrates titrated samples mixed with cfDNA and WBC serum, according to example implementations.

FIG. 19 illustrates titrated samples with calculated Mahalanobis distance, according to the second methodology, according to example implementations.

FIG. 20 illustrates titrated samples quantified for WBC contamination, according to example implementations.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

Early detection and classification of cancer is an important technology. Being able to detect cancer before it becomes symptomatic is beneficial to all parties involved, including patients, doctors, and loved ones. For patients, early cancer detection allows them a greater chance of a beneficial outcome; for doctors, early cancer detection allows more pathways of treatment that may lead to a beneficial outcome; for loved ones, early cancer detection increases the likelihood of not losing their friends and family to the disease.

Recently, early cancer detection technology has progressed towards analyzing genetic fragments (e.g., DNA) in a person's, for example, blood to determine if any of those genetic fragments originate from cancer cells. These new techniques allow doctors to identify a cancer presence in a patient that may not be detectable otherwise, e.g., in conventional screening processes. For instance, consider the example of a person at high risk for breast cancer. Traditionally, this person will regularly visit their doctor for a mammogram, which creates an image of their breast tissue (e.g., taking x-ray images) that a doctor uses to identify cancerous tissue. Unfortunately, with even the highest resolution mammograms, doctors are only able to identify tumors once they are approximately a millimeter in size. This means that the cancer has been present for some time in the person and has gone undiagnosed and untreated. Visual determinations like this are typical for most cancers—that is, only being identifiable once it has grown to a sufficient size and has become identifiable with some sort of imaging technology.

Cancer detection using analysis of genetic fragments in a patient's, e.g., blood alleviates this issue. To illustrate, cancer cells will start sloughing DNA fragments into a person's bloodstream as soon as they form. This occurs when there are very few of the cancer cells, and before they would be visible with imaging techniques. With the appropriate methods, therefore, a system that analyzes DNA fragments in the bloodstream could identify cancer presence in a person based on sloughed cancer DNA fragments, and, more importantly, they system could do so before the cancer is identifiable using more traditional cancer detection techniques.

Cancer detection based on the analysis of DNA fragments is enabled by next-generation sequencing (“NGS”) techniques. NGS, broadly, is a group of technologies that allows for high throughput sequencing of genetic material. As discussed in greater detail herein, NGS largely consists of (1) sample preparation, (2) DNA sequencing, and (3) data analysis. Sample preparation is the laboratory methods necessary to prepare DNA fragments for sequencing, sequencing is the process of reading the ordered nucleotides in the samples, and data analysis is processing and analyzing the genetic information in the sequencing data to identify cancer presence.

While these steps of NGS may help enable early cancer detection, they also introduce their own complex, detrimental problems to cancer detection and, therefore, any improvements to sample preparation, DNA sequencing, and/or data analysis, including the pre-processing, algorithmic processing, and summary or presentation of predications or conclusions, results in an improvement to cancer detection technologies and early cancer detection more generally.

To illustrate, as an example, problems introduced in (1) sample preparation include DNA sample quality, sample contamination, fragmentation bias, and accurate indexing. Remedying these problems would yield better genetic data for cancer detection. Similarly, problems introduced in (2) sequencing include, for example, errors in accurate transcribing of fragments (e.g., reading an “A” instead of a “C”, etc.), incorrect or difficult fragment assembly and overlap, disparate coverage uniformity, sequencing depth vs. cost vs. specificity, and insufficient sequencing length. Again, remedying any of these problems would yield improved genetic data for cancer detection.

The problems in (3) data analysis are the most daunting and complex. The introduced challenges stem from the vast amounts of data created by NGS sequencing techniques. The created genetic datasets are typically on the order of terabytes, and effectively and efficiently analyzing that amount of data is both procedurally and computationally demanding. For instance, analyzing NGS sequencing involves several baseline processing steps such as, e.g., aligning reads to one another, aligning and mapping reads to a reference genome, identifying and calling variant genes, identifying and calling abnormally methylated genes, generating functional annotations, etc. Performing any of these processes on terabytes of genetic data is computationally expensive for even the most powerful of computer architectures, and completely impossible for a normal human mind. Additionally, with the genetic sequencing data derived from the error-prone processes of sample preparation and sequence reading, large portions of the resulting genetic data may be low-quality or unusable for cancer identification. For example, large amounts of the genetic data may include contaminated samples, transcription errors, mismatched regions, overrepresented regions, etc. and may be unsuitable for high accuracy cancer detection. Identifying and accounting for low quality genetic data across the vast amount of genetic data obtained from NGS sequencing is also procedurally and computationally rigorous to accomplish and is also not practically performable by a human mind. Overall, any process created that leads to more efficient processing of large array sequencing data would be an improvement to cancer detection using NGS sequencing.

Finally, and perhaps most importantly, accurate identification of informative DNA from NGS data to identify a cancer presence is also difficult (much more in the early cancer detection context). To be effective, algorithms are sought to compensate for, e.g., errors generated by sample preparation and sequencing, and to overcome the large-scale data analysis problems accompanying NGS techniques. That is, designing a machine learning model or models, or other computational processing algorithms, that enable early cancer detection based on next generation sequencing techniques must be configured to account for the problems that those techniques create. Some of those techniques and models are discussed hereinbelow and particular improvements to state-of-the-art techniques and models are further discussed.

One particular challenge arises in the sample preparation phase with WBC-shed DNA. The cancer signal is typically muddled with the WBC-shed signal, which may confound cancer detection when analyzed in conjunction with the cfDNA in the plasma. This confounding of the signals may lead to false positive calls of cancer. The computer models implemented in WBC contamination detection aim to assess whether a sample is contaminated by the WBC-shed DNA and may further determine a level of contamination. Upon detection of a WBC-contaminated sample, the analytics system can perform remedial measures. For example, the analytics system may remove such sample from use in training of the cancer classification model, thereby improving the accuracy and precision of the cancer classification model. To expand further, the analytics system may perform WBC contamination detection from an initial set of training samples to determine any WBC-contaminated samples to remove form the set of training samples. The filtered set of training samples (free of WBC contamination) may be used for training of models. As another example, the analytics system may withhold a contaminated test sample from cancer classification, thereby reducing the false-positive rate of prediction of cancer. Upon detection of a contaminated sample, further remedial measures may be taken to address the contamination. Remedial measures may include identifying a source of the contamination through performing tests on varying conditions of the sample preparation process to pinpoint conditions that contribute to the WBC contamination. Remedying sources of contamination thereby improves the physical assaying process and the sample processing. Upon identifying the source of contamination, action be undertaken to remove the contamination source, or minimize the contamination in the sample processing workflow. Another remedial measure may be physically discarding the sample.

The training of the machine-learned models described herein (such as the contamination models, the cancer classifier, any other neural network, and any other model referenced herein) include the performance of one or more non-mathematical operations or implementation of non-mathematical functions at least in part by a machine or computing system, examples of which include but are not limited to data loading operations, data storage operations, data toggling or modification operations, non-transitory computer-readable storage medium modification operations, metadata removal or data cleansing operations, data compression operations, protein structure modification operations, image modification operations, noise application operations, noise removal operations, and the like. Accordingly, the training of the machine-learned models described herein may be based on or may involve mathematical concepts, but is not simply limited to the performance of a mathematical calculation, a mathematical operation, or an act of calculating a variable or number using mathematical methods.

Likewise, it should be noted that the training of these models described herein cannot be practically performed in the human mind. The models are innately complex including vast amounts of weights and parameters associated through one or more complex functions. Training and/or deployment of such models involve so great a number of operations that it is not feasibly performable by the human mind alone, nor with the assistance of pen and paper. In such embodiments, the operations may number in the hundreds, thousands, tens of thousands, hundreds of thousands, millions, billions, or trillions. Moreover, the training data may include hundreds, thousands, tens of thousands, hundreds of thousands, millions, or billions of sequence reads, each sequence read may further include anywhere from hundreds up to thousands of nucleotides. Accordingly, such models are necessarily rooted in computer-technology for their implementation and use.

### I.A. Cancer Classification Workflow

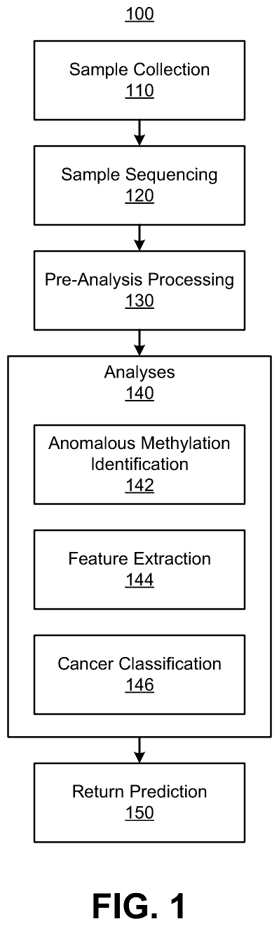

FIG. 1 is an exemplary flowchart describing an overall workflow 100 of cancer classification of a sample, according to one or more embodiments. The workflow 100 is by one or more entities, e.g., including a healthcare provider, a sequencing device, an analytics system, etc. Objectives of the workflow include detecting and/or monitoring cancer in individuals. From a healthcare standpoint, the workflow 100 can serve to supplement other existing cancer diagnostic tools. The workflow 100 may serve to provide early cancer detection and/or routine cancer monitoring to better inform treatment plans for individuals diagnosed with cancer. The overall workflow 100 may include additional/fewer steps than those shown in FIG. 1.

A healthcare provider performs sample collection 110. An individual to undergo cancer classification visits their healthcare provider. The healthcare provider collects the sample for performing cancer classification. Examples of biological samples include, but are not limited to, tissue biopsy, blood, whole blood, plasma, serum, urine, cerebrospinal fluid, fecal, saliva, sweat, tears, pleural fluid, pericardial fluid, or peritoneal fluid of the subject. In one or more embodiments, collecting of the sample is minimally invasive or non-invasive. The sample includes genetic material belonging to the individual, which may be extracted and sequenced for cancer classification. Once the sample is collected, the sample is provided to a sequencing device. Along with the sample, the healthcare provider may collect other information relating to the individual, e.g., biological sex, age, race, smoking status, other health metrics, any prior diagnoses, etc.

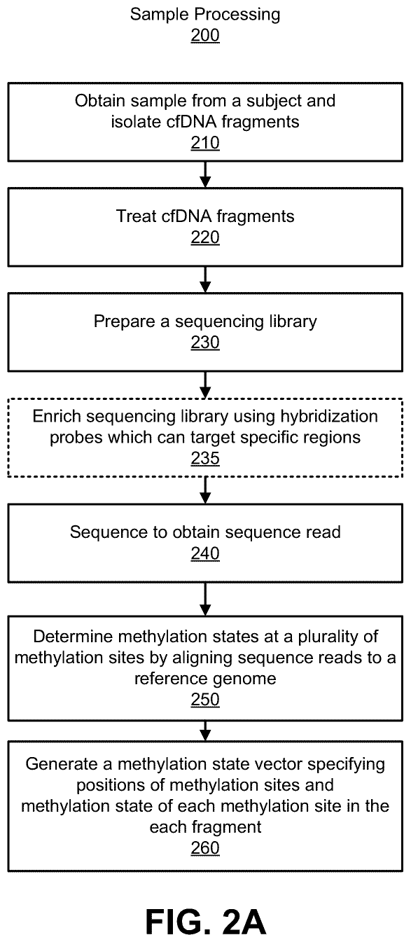

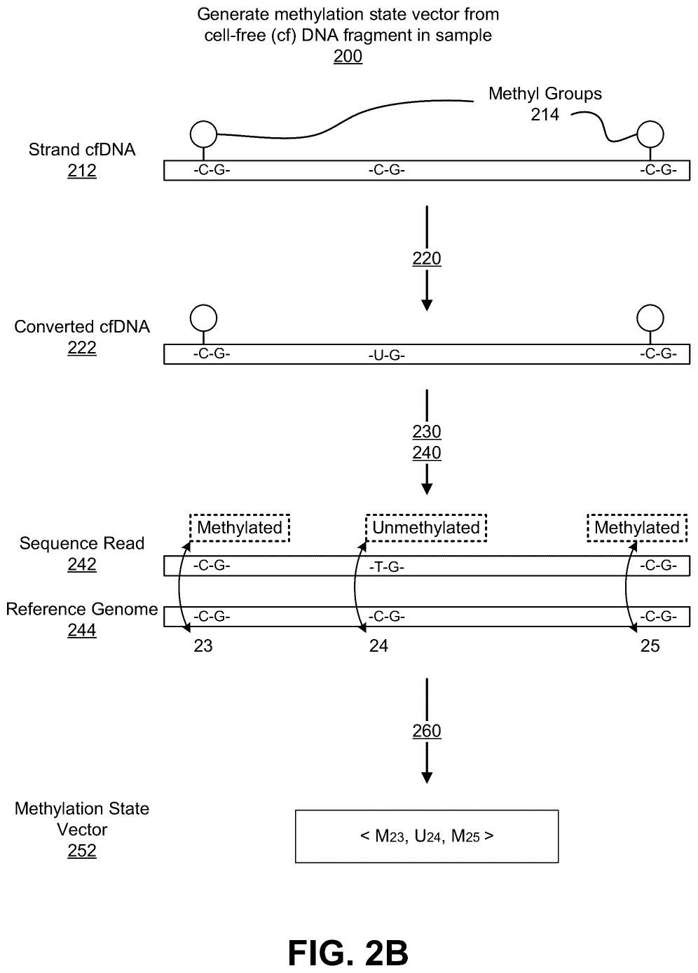

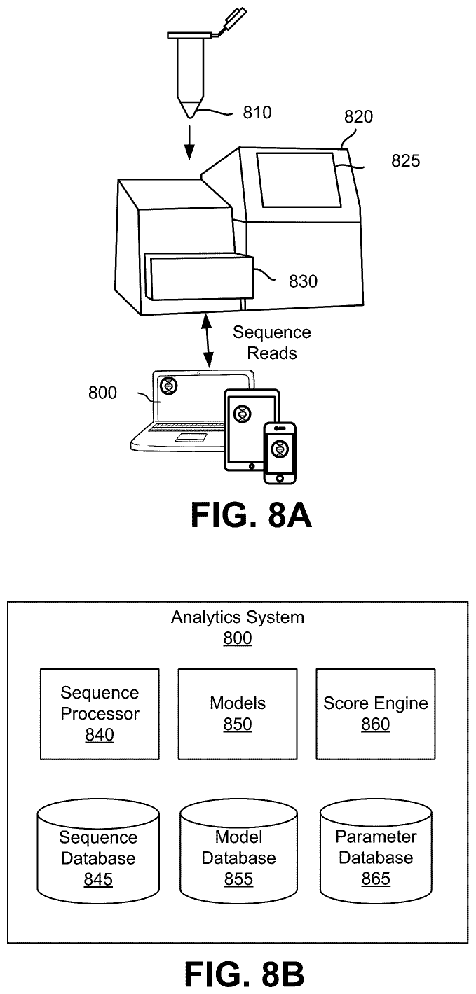

A sequencing device performs sample sequencing 120. A lab clinician may perform one or more processing steps to the sample in preparation of sequencing. Once prepared, the clinician loads the sample in the sequencing device. An example of devices utilizes in sequencing is further described in conjunction with FIGS. 8A & 8B. The sequencing device generally extracts and isolates fragments of nucleic acid that are sequenced to determine a sequence of nucleobases corresponding to the fragments. Sequencing may also include amplification of nucleic material. Different sequencing processes include Sanger sequencing, fragment analysis, and other next-generation sequencing techniques. Next-generation sequencing is capable of yielding high-throughput sequencing data, e.g., 10,000, 100,000, 1,000,000, or 10,000,000 sequence reads, and each sequence read may be of length 50 bp, 100 bp, 150 bp, 200 bp, 250 bp, 300 bp, etc. Accordingly, the high-throughput sequencing data is of a size that it is impractical for a human mind to analyze. Sequencing may be whole-genome sequencing or targeted sequencing with a target panel. In context of DNA methylation, bisulfite sequencing (e.g., further described in FIGS. 2A & 2B) can determine methylations status through bisulfite conversion of unmethylated cytosines at CpG sites. Sample sequencing 120 yields sequences for a plurality of nucleic acid fragments in the sample. In one or more embodiments, the sequences may include methylation state vectors, wherein each methylation state vector describes the methylation statuses for CpG sites on a fragment.

An analytics system performs pre-analysis processing 130. An example analytics system is described in FIG. 8B. Pre-analysis processing 130 may include, but not limited to, de-duplication of sequence reads, determining metrics relating to coverage, determining whether the sample is contaminated (including determining WBC contamination), removal of contaminated fragments, calling sequencing error, etc.

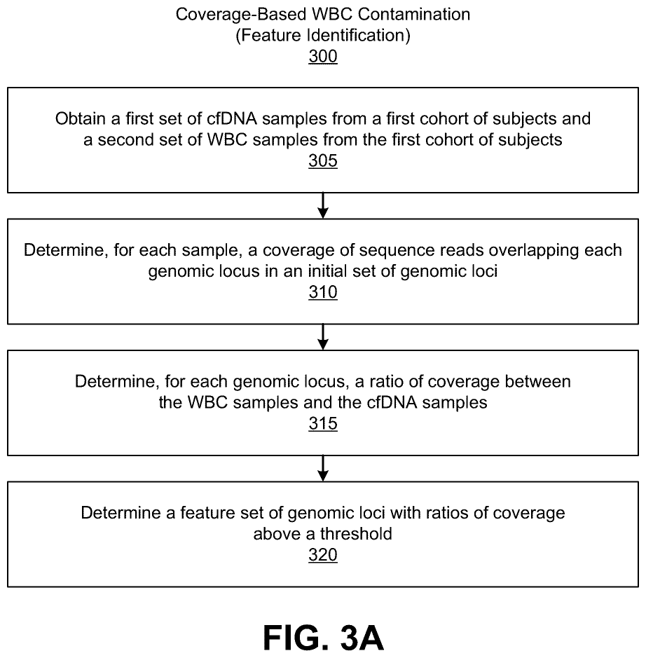

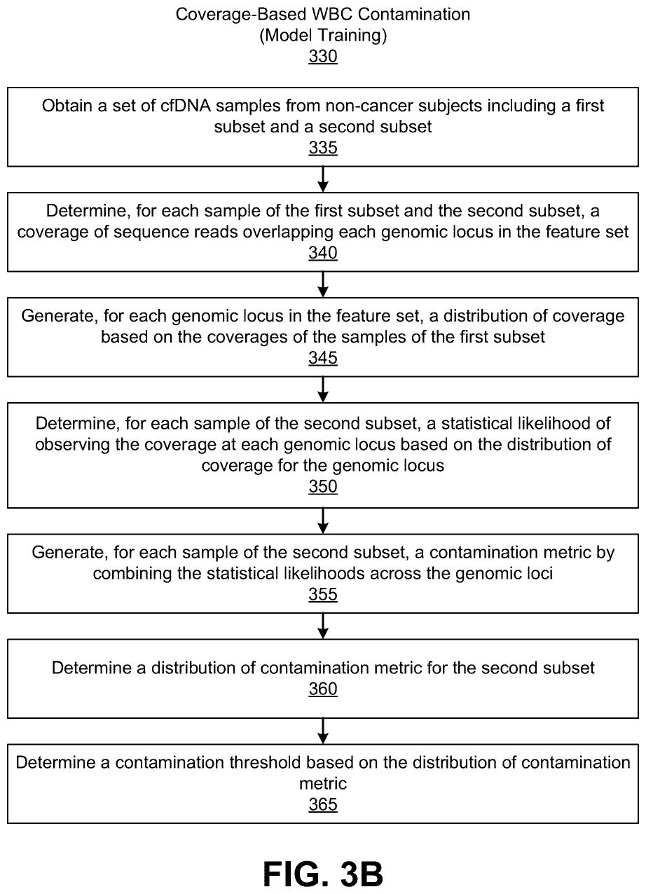

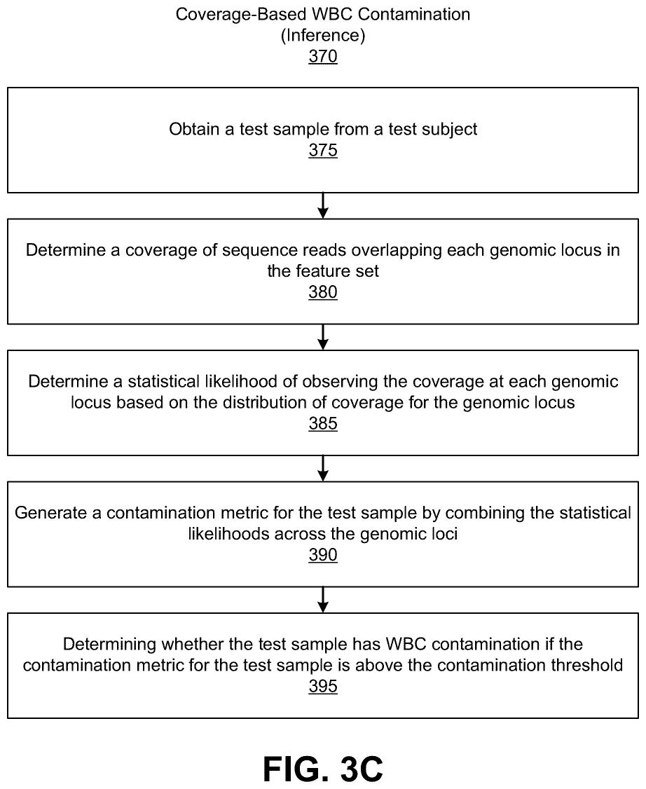

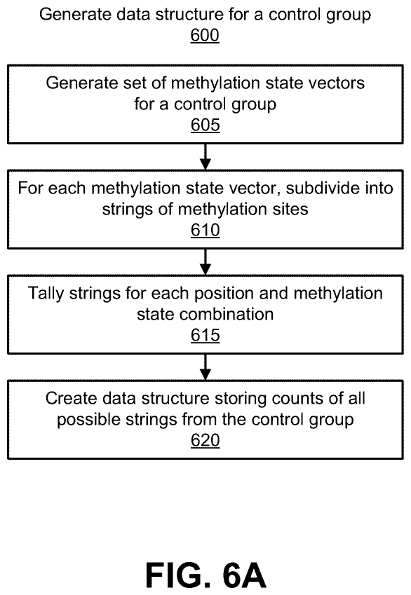

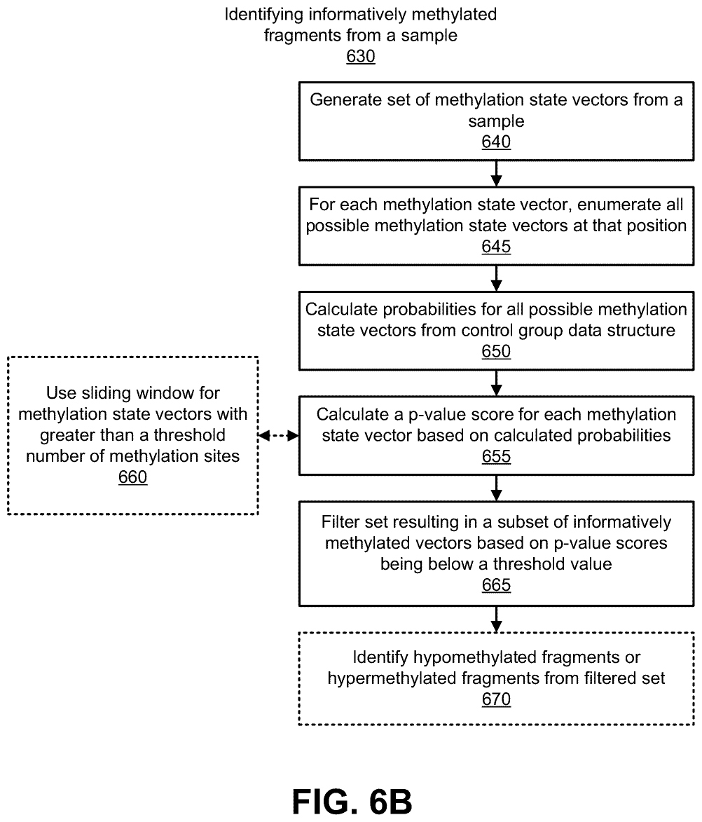

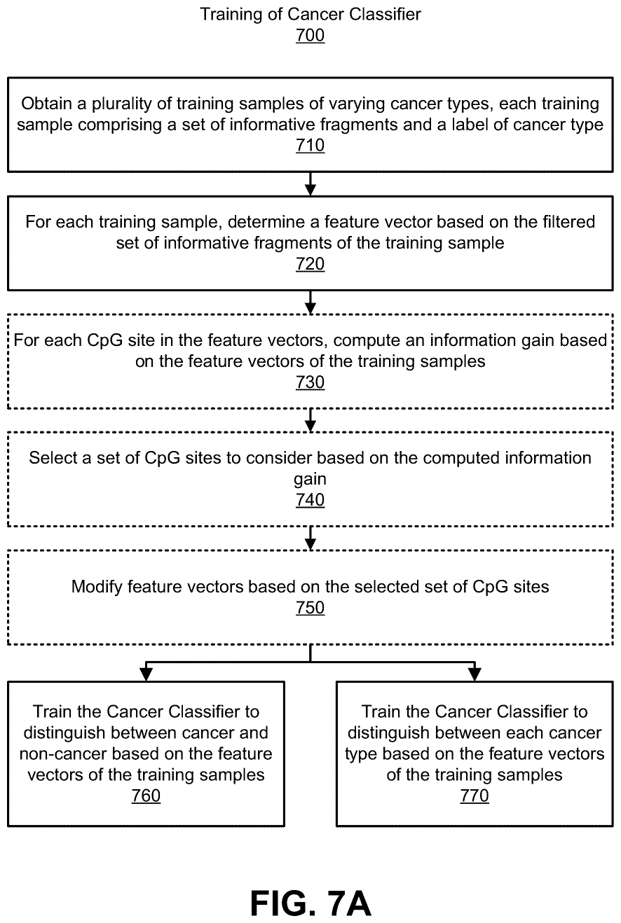

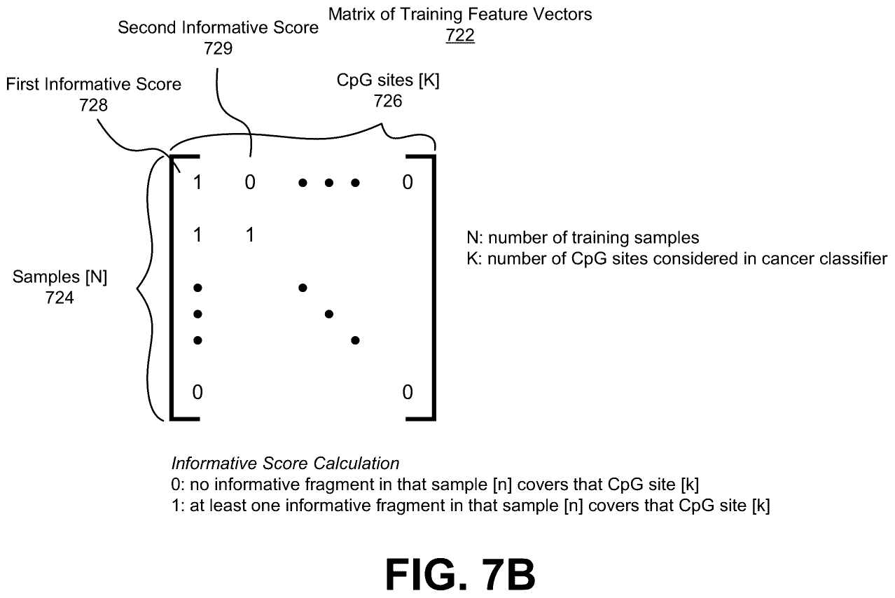

The analytics system performs one or more analyses 140. The analyses are statistical analyses or application of one or more trained models to predict at least a cancer status of the individual from whom the sample is derived. Different genetic features may be evaluated and considered, such as methylation of CpG sites, single nucleotide polymorphisms (SNPs), insertions or deletions (indels), other types of genetic mutation, etc. In context of methylation, analyses 140 may include contamination detection 142 (e.g., further described in FIGS. 3A-3C, 4A-4C, and 5A & 5B), informative methylation identification 144 (e.g., further described in FIGS. 6A & 6B), feature extraction 146 (e.g., further described 7A, and 7B), and applying a cancer classifier 148 to determine a cancer prediction (e.g., further described in FIGS. 7A & 7B). The cancer classifier 148 inputs the extracted features to determine a cancer prediction. The cancer prediction may be a label or a value. The label may indicate a particular cancer state, e.g., binary labels can indicate presence or absence of cancer, multiclass labels can indicate one or more cancer types from a plurality of cancer types that are screened for. The value may indicate a likelihood of a particular cancer state, e.g., a likelihood of cancer, and/or a likelihood of a particular cancer type.

The analytics system returns the prediction 150 to the healthcare provider. The healthcare provider may establish or adjust a treatment plan based on the cancer prediction. Optimization of treatment is further described in Section IV.C. Treatment. In some embodiments, the analytics system may leverage the cancer classification workflow for prognosis determination, treatment personalization, evaluation of treatment, monitoring cancer status, etc.

### I.B. Methylation Overview

In accordance with the present description, cfDNA fragments from an individual are treated, for example by converting unmethylated cytosines to uracils, sequenced and the sequence reads compared to a reference genome to identify the methylation states at specific CpG sites within the DNA fragments. Each CpG site may be methylated or unmethylated. Identification of informatively methylated fragments, in comparison to healthy individuals, may provide insight into a subject's cancer status. As is well known in the art, DNA methylation anomalies (compared to healthy controls) can cause different effects, which may contribute to cancer. Various challenges arise in the identification of informatively methylated cfDNA fragments. First off, determining a DNA fragment to be informatively methylated can hold weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be informatively methylated. On another note, methylation of a cytosine at a CpG site can causally influence methylation at a subsequent CpG site. To encapsulate this dependency can be another challenge in itself.

Methylation can typically occur in deoxyribonucleic acid (DNA) when a hydrogen atom on the pyrimidine ring of a cytosine base is converted to a methyl group, forming 5-methylcytosine. In particular, methylation can occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Informative DNA methylation can be identified as hypermethylation or hypomethylation, both of which may be indicative of cancer status. Throughout this disclosure, hypermethylation and hypomethylation can be characterized for a DNA fragment, if the DNA fragment comprises more than a threshold number of CpG sites with more than a threshold percentage of those CpG sites being methylated or unmethylated.

The principles described herein can be equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein can be the same, and consequently the inventive concepts described herein can be applicable to those other forms of methylation.

### I.C. Definitions

The term “cell free nucleic acid” or “cfNA” refers to nucleic acid fragments that circulate in an individual's body (e.g., blood) and originate from one or more healthy cells and/or from one or more unhealthy cells (e.g., cancer cells). The term “cell free DNA,” or “cfDNA” refers to deoxyribonucleic acid fragments that circulate in an individual's body (e.g., blood). Additionally, cfNAs or cfDNA in an individual's body may come from other non-human sources.

The term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, and which may be released into a bodily fluid of an individual (e.g., blood, sweat, urine, or saliva) as result of biological processes such as apoptosis or necrosis of dying cells or actively released by viable tumor cells.

The term “DNA fragment,” “fragment,” or “DNA molecule” may generally refer to any deoxyribonucleic acid fragments, i.e., cfDNA, gDNA, ctDNA, etc.

The term “informative fragment,” “informatively methylated fragment,” or “fragment with an informative methylation pattern” refers to a fragment that has anomalous methylation of CpG sites. Anomalous methylation of a fragment may be determined using probabilistic models to identify unexpectedness of observing a fragment's methylation pattern in a control group.

The term “unusual fragment with extreme methylation” or “UFXM” refers to a hypomethylated fragment or a hypermethylated fragment. A hypomethylated fragment and a hypermethylated fragment refers to a fragment with at least some number of CpG sites (e.g., 5) that have over some threshold percentage (e.g., 90%) of methylation or unmethylation, respectively.

The term “informative score” refers to a score for a CpG site based on a number of informative fragments (or, in some embodiments, UFXMs) from a sample overlaps that CpG site. The informative score is used in context of featurization of a sample for classification.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

As used herein, the term “biological sample,” “patient sample,” or “sample” refers to any sample taken from a subject, which can reflect a biological state associated with the subject, and that includes cell-free DNA. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, pericardial fluid, peritoneal fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. A biological sample can include any tissue or material derived from a living or dead subject. A biological sample can be a cell-free sample. A biological sample can comprise a nucleic acid (e.g., DNA or RNA) or a fragment thereof.

The term “nucleic acid” can refer to deoxyribonucleic acid (DNA), ribonucleic acid (RNA) or any hybrid or fragment thereof. The nucleic acid in the sample can be a cell-free nucleic acid. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragment sequences obtained from sequencing a sample from the subject. A reference genome can refer to a haploid or diploid genome to which nucleic acid fragment sequences from the biological sample and a constitutional sample can be aligned and compared. An example of a constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the term “cancer” or “tumor” refers to an abnormal mass of tissue in which the growth of the mass surpasses and is not coordinated with the growth of normal tissue.

As used herein, the phrase “healthy,” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that's not cytosine; however, these are rarer occurrences. Informative cfDNA methylation can be identified as hypermethylation or hypomethylation, both of which may be indicative of cancer status. DNA methylation anomalies (compared to healthy controls) can cause different effects, which may contribute to cancer. The principles described herein are equally applicable for the detection of methylation in a CpG context and non-CpG context, including non-cytosine methylation. Further, the methylation state vectors may contain elements that are generally vectors of sites where methylation has or has not occurred (even if those sites are not CpG sites specifically).

As used interchangeably herein, the term “methylation fragment” or “nucleic acid methylation fragment” refers to a sequence of methylation states for each CpG site in a plurality of CpG sites, determined by a methylation sequencing of nucleic acids (e.g., a nucleic acid molecule and/or a nucleic acid fragment). In a methylation fragment, a location and methylation state for each CpG site in the nucleic acid fragment is determined based on the alignment of the sequence reads (e.g., obtained from sequencing of the nucleic acids) to a reference genome. A nucleic acid methylation fragment comprises a methylation state of each CpG site in a plurality of CpG sites (e.g., a methylation state vector), which specifies the location of the nucleic acid fragment in a reference genome (e.g., as specified by the position of the first CpG site in the nucleic acid fragment using a CpG index, or another similar metric) and the number of CpG sites in the nucleic acid fragment. Alignment of a sequence read to a reference genome, based on a methylation sequencing of a nucleic acid molecule, can be performed using a CpG index. As used herein, the term “CpG index” refers to a list of each CpG site in the plurality of CpG sites (e.g., CpG 1, CpG 2, CpG 3, etc.) in a reference genome, such as a human reference genome, which can be in electronic format. The CpG index further comprises a corresponding genomic location, in the corresponding reference genome, for each respective CpG site in the CpG index. Each CpG site in each respective nucleic acid methylation fragment is thus indexed to a specific location in the respective reference genome, which can be determined using the CpG index.

As used herein, the term “true positive” (TP) refers to a subject having a condition. “True positive” can refer to a subject that has a tumor, a cancer, a pre-cancerous condition (e.g., a pre-cancerous lesion), a localized or a metastasized cancer, or a non-malignant disease. “True positive” can refer to a subject having a condition and is identified as having the condition by an assay or method of the present disclosure. As used herein, the term “true negative” (TN) refers to a subject that does not have a condition or does not have a detectable condition. True negative can refer to a subject that does not have a disease or a detectable disease, such as a tumor, a cancer, a pre-cancerous condition (e.g., a pre-cancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 450 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the terms “sequencing” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequencing depth,” is interchangeably used with the term “coverage” and refers to the number of times a locus is covered by a consensus sequence read corresponding to a unique nucleic acid target molecule aligned to the locus; e.g., the sequencing depth is equal to the number of unique nucleic acid target molecules covering the locus. The locus can be as small as a nucleotide, or as large as a chromosome arm, or as large as an entire genome. Sequencing depth can be expressed as “Yx”, e.g., 50×, 100×, etc., where “Y” refers to the number of times a locus is covered with a sequence corresponding to a nucleic acid target; e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a locus or a haploid genome, or a whole genome, respectively, is sequenced. When a mean depth is quoted, the actual depth for different loci included in the dataset can span over a range of values. Ultra-deep sequencing can refer to at least 100× in sequencing depth at a locus.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. In some embodiments, a subject is a male or female of any stage (e.g., a man, a woman or a child). A subject from whom a sample is taken, or is treated by any of the methods or compositions described herein can be of any age and can be an adult, infant or child.

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “genomic” refers to a characteristic of the genome of an organism. Examples of genomic characteristics include, but are not limited to, those relating to the primary nucleic acid sequence of all or a portion of the genome (e.g., the presence or absence of a nucleotide polymorphism, indel, sequence rearrangement, mutational frequency, etc.), the copy number of one or more particular nucleotide sequences within the genome (e.g., copy number, allele frequency fractions, single chromosome or entire genome ploidy, etc.), the epigenetic status of all or a portion of the genome (e.g., covalent nucleic acid modifications such as methylation, histone modifications, nucleosome positioning, etc.), the expression profile of the organism's genome (e.g., gene expression levels, isotype expression levels, gene expression ratios, etc.).

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

### I.D. Example Analytics System

FIG. 8A is an exemplary flowchart of devices for sequencing nucleic acid samples according to one or more embodiments. This illustrative flowchart includes devices such as a sequencer 820 and an analytics system 800. The sequencer 820 and the analytics system 800 may work in tandem to perform one or more steps in the processes.

In various embodiments, the sequencer 820 receives an enriched nucleic acid sample 810. As shown in FIG. 8A, the sequencer 820 can include a graphical user interface 825 that enables user interactions with particular tasks (e.g., initiate sequencing or terminate sequencing) as well as one more loading stations 830 for loading a sequencing cartridge including the enriched fragment samples and/or for loading necessary buffers for performing the sequencing assays. Therefore, once a user of the sequencer 820 has provided the necessary reagents and sequencing cartridge to the loading station 830 of the sequencer 820, the user can initiate sequencing by interacting with the graphical user interface 825 of the sequencer 820. Once initiated, the sequencer 820 performs the sequencing and outputs the sequence reads of the enriched fragments from the nucleic acid sample 810.

In some embodiments, the sequencer 820 is communicatively coupled with the analytics system 800. The analytics system 800 includes some number of computing devices used for processing the sequence reads for various applications such as assessing methylation status at one or more CpG sites, variant calling or quality control. The sequencer 820 may provide the sequence reads in a BAM file format to the analytics system 800. The analytics system 800 can be communicatively coupled to the sequencer 820 through a wireless, wired, or a combination of wireless and wired communication technologies. Generally, the analytics system 800 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 800 may label a sequence read with one or more genes that align to the sequence read. In one embodiment, fragment length (or size) is be determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome can represent the likely location within the reference genome to which the nucleic acid fragment corresponds. An output file having SAM (sequence alignment map) format or BAM (binary) format may be generated and output for further analysis.

Referring now to FIG. 8B, FIG. 8B is a block diagram of an analytics system 800 for processing DNA samples according to one embodiment. The analytics system implements one or more computing devices for use in analyzing DNA samples. The analytics system 800 includes a sequence processor 840, sequence database 845, model database 855, models 850, parameter database 865, and score engine 860. In some embodiments, the analytics system 800 performs some or all of the processes described throughout this disclosure.

The sequence processor 840 generates methylation state vectors for fragments from a sample. At each CpG site on a fragment, the sequence processor 840 generates a methylation state vector for each fragment specifying a location of the fragment in the reference genome, a number of CpG sites in the fragment, and the methylation state of each CpG site in the fragment whether methylated, unmethylated, or indeterminate via the process 200 of FIG. 2A. The sequence processor 840 may store methylation state vectors for fragments in the sequence database 845. Data in the sequence database 845 may be organized such that the methylation state vectors from a sample are associated to one another.

Further, multiple different models 850 may be stored in the model database 855 or retrieved for use with test samples. In one example, a model is a trained cancer classifier for determining a cancer prediction for a test sample using a feature vector derived from informative fragments. The training and use of the cancer classifier will be further discussed in conjunction with Section IV. Cancer Classifier for Determining Cancer. The analytics system 800 may train the one or more models 850 and store various trained parameters in the parameter database 865. The analytics system 800 stores the models 850 along with functions in the model database 855.

During inference, the score engine 860 uses the one or more models 850 to return outputs. The score engine 860 accesses the models 850 in the model database 855 along with trained parameters from the parameter database 865. According to each model, the score engine receives an appropriate input for the model and calculates an output based on the received input, the parameters, and a function of each model relating the input and the output. In some use cases, the score engine 860 further calculates metrics correlating to a confidence in the calculated outputs from the model. In other use cases, the score engine 860 calculates other intermediary values for use in the model.

### II. Sample Sequencing & Processing

### II.A. Generating Methylation State Vectors for DNA Fragments

FIG. 2A is an exemplary flowchart describing a process 200 of sequencing a fragment of cfDNA to obtain a methylation state vector, according to one or more embodiments. In order to analyze DNA methylation, an analytics system first obtains 210 a sample from an individual comprising a plurality of cfDNA molecules. In additional embodiments, the process 200 may be applied to sequence other types of DNA molecules. The process 200 is an embodiment of sample sequencing 120 of FIG. 1.

From the sample, the analytics system can isolate 210 each cfDNA molecule. The cfDNA molecules can be treated 220 to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™-Gold, EZ DNA Methylation™-Direct or an EZ DNA Methylation™-Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, MA).

From the converted cfDNA molecules, a sequencing library can be prepared 230. During library preparation, unique molecular identifiers (UMI) can be added to the nucleic acid molecules (e.g., DNA molecules) through adapter ligation. The UMIs can be short nucleic acid sequences (e.g., 4-10 base pairs) that are added to ends of DNA fragments (e.g., DNA molecules fragmented by physical shearing, enzymatic digestion, and/or chemical fragmentation) during adapter ligation. UMIs can be degenerate base pairs that serve as a unique tag that can be used to identify sequence reads originating from a specific DNA fragment. During PCR amplification following adapter ligation, the UMIs can be replicated along with the attached DNA fragment. This can provide a way to identify sequence reads that came from the same original fragment in downstream analysis.

Optionally, the sequencing library may be enriched 235 for cfDNA molecules, or genomic regions, that are informative for cancer status using a plurality of hybridization probes. The hybridization probes are short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified CpG sites of interest to the researcher. Hybridization probes can be tiled across one or more target sequences at a coverage of 1×, 2×, 3×, 4×, 5×, 6×, 7×, 8×, 9×, 10×, or more than 10×. For example, hybridization probes tiled at a coverage of 2× comprises overlapping probes such that each portion of the target sequence is hybridized to 2 independent probes. Hybridization probes can be tiled across one or more target sequences at a coverage of less than 1×.

In one embodiment, the hybridization probes are designed to enrich for DNA molecules that have been treated (e.g., using bisulfite) for conversion of unmethylated cytosines to uracils. During enrichment, hybridization probes (also referred to herein as “probes”) can be used to target and pull down nucleic acid fragments informative for the presence or absence of cancer (or disease), cancer status, or a cancer classification (e.g., cancer class or tissue of origin). The probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10 s, 100 s, or 1000 s of base pairs. The probes can be designed based on a methylation site panel. The probes can be designed based on a panel of targeted genes to analyze particular mutations or target regions of the genome (e.g., of the human or another organism) that are suspected to correspond to certain cancers or other types of diseases. Moreover, the probes may cover overlapping portions of a target region.

Once prepared, the sequencing library or a portion thereof can be sequenced 240 to obtain a plurality of sequence reads. The sequence reads may be in a computer-readable, digital format for processing and interpretation by computer software. The sequence reads may be aligned to a reference genome to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. A sequence read can be comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome can represent the likely location within the reference genome to which the nucleic acid fragment corresponds. An output file having SAM (sequence alignment map) format or BAM (binary) format may be generated and output for further analysis such as methylation state determination.

From the sequence reads, the analytics system determines 250 a location and methylation state for each CpG site based on alignment to a reference genome. The analytics system generates 260 a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states can be states of methylated and unmethylated; whereas, an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses.

FIG. 2B is an exemplary illustration of the process 200 of FIG. 2A of sequencing a cfDNA molecule to obtain a methylation state vector, according to one or more embodiments. As an example, the analytics system receives a cfDNA molecule 212 that, in this example, contains three CpG sites. As shown, the first and third CpG sites of the cfDNA molecule 212 are methylated 214. During the treatment step 220, the cfDNA molecule 212 is converted to generate a converted cfDNA molecule 222. During the treatment 220, the second CpG site which was unmethylated has its cytosine converted to uracil. However, the first and third CpG sites were not converted.

After conversion, a sequencing library 230 is prepared and sequenced 240 to generate a sequence read 242. The analytics system aligns 250 the sequence read 242 to a reference genome 244. The reference genome 244 provides the context as to what position in a human genome the fragment cfDNA originates from. In this simplified example, the analytics system aligns 250 the sequence read 242 such that the three CpG sites correlate to CpG sites 23, 24, and 25 (arbitrary reference identifiers used for convenience of description). The analytics system can thus generate information both on methylation status of all CpG sites on the cfDNA molecule 212 and the position in the human genome that the CpG sites map to. As shown, the CpG sites on sequence read 242 which are methylated are read as cytosines. In this example, the cytosines appear in the sequence read 242 only in the first and third CpG site which allows one to infer that the first and third CpG sites in the original cfDNA molecule are methylated. Whereas, the second CpG site can be read as a thymine (U is converted to T during the sequencing process), and thus, one can infer that the second CpG site is unmethylated in the original cfDNA molecule. With these two pieces of information, the methylation status and location, the analytics system generates 260 a methylation state vector 252 for the fragment cfDNA 212. In this example, the resulting methylation state vector 252 is <M₂₃, U₂₄, M₂₅>, wherein M corresponds to a methylated CpG site, U corresponds to an unmethylated CpG site, and the subscript number corresponds to a position of each CpG site in the reference genome.

One or more alternative sequencing methods can be used for obtaining sequence reads from nucleic acids in a biological sample. The one or more sequencing methods can comprise any form of sequencing that can be used to obtain a number of sequence reads measured from nucleic acids (e.g., cell-free nucleic acids), including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single-molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and Nanopore sequencing can also be used to obtain sequence reads from the nucleic acids (e.g., cell-free nucleic acids) in the biological sample. Sequencing-by-synthesis and reversible terminator-based sequencing (e.g., Illumina's Genome Analyzer; Genome Analyzer II; HISEQ 2000; HISEQ 4500 (Illumina, San Diego Calif.)) can be used to obtain sequence reads from the cell-free nucleic acid obtained from a biological sample of a training subject in order to form the genotypic dataset. Millions of cell-free nucleic acid (e.g., DNA) fragments can be sequenced in parallel. In one example of this type of sequencing technology, a flow cell is used that contains an optically transparent slide with eight individual lanes on the surfaces of which are bound oligonucleotide anchors (e.g., adaptor primers). A cell-free nucleic acid sample can include a signal or tag that facilitates detection. The acquisition of sequence reads from the cell-free nucleic acid obtained from the biological sample can include obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

The one or more sequencing methods can comprise a whole-genome sequencing assay. A whole-genome sequencing assay can comprise a physical assay that generates sequence reads for a whole genome or a substantial portion of the whole genome which can be used to determine large variations such as copy number variations or copy number aberrations. Such a physical assay may employ whole-genome sequencing techniques or whole-exome sequencing techniques. A whole-genome sequencing assay can have an average sequencing depth of at least 1×, 2×, 3×, 4×, 5×, 6×, 7×, 8×, 9×, 10×, at least 20×, at least 30×, or at least 40× across the genome of the test subject. In some embodiments, the sequencing depth is about 30,000×. The one or more sequencing methods can comprise a targeted panel sequencing assay. A targeted panel sequencing assay can have an average sequencing depth of at least 50,000×, at least 55,000×, at least 60,000×, or at least 70,000× sequencing depth for the targeted panel of genes. The targeted panel of genes can comprise between 450 and 500 genes. The targeted panel of genes can comprise a range of 500±5 genes, a range of 500±10 genes, or a range of 500±25 genes.

The one or more sequencing methods can comprise paired-end sequencing. The one or more sequencing methods can generate a plurality of sequence reads. The plurality of sequence reads can have an average length ranging between 10 and 700, between 50 and 400, or between 100 and 300. The one or more sequencing methods can comprise a methylation sequencing assay. The methylation sequencing can be i) whole-genome methylation sequencing or ii) targeted DNA methylation sequencing using a plurality of nucleic acid probes. For example, the methylation sequencing is whole-genome bisulfite sequencing (e.g., WGBS). The methylation sequencing can be a targeted DNA methylation sequencing using a plurality of nucleic acid probes targeting the most informative regions of the methylome, a unique methylation database and prior prototype whole-genome and targeted sequencing assays.

The methylation sequencing can detect one or more 5-methylcytosine (5mC) and/or 5-hydroxymethylcytosine (5hmC) in respective nucleic acid methylation fragments. The methylation sequencing can comprise conversion of one or more unmethylated cytosines or one or more methylated cytosines, in respective nucleic acid methylation fragments, to a corresponding one or more uracils. The one or more uracils can be detected during the methylation sequencing as one or more corresponding thymines. The conversion of one or more unmethylated cytosines or one or more methylated cytosines can comprise a chemical conversion, an enzymatic conversion, or combinations thereof.

For example, bisulfite conversion involves converting cytosine to uracil while leaving methylated cytosines (e.g., 5-methylcytosine or 5-mC) intact. In some DNA, about 95% of cytosines may not methylated in the DNA, and the resulting DNA fragments may include many uracils which are represented by thymines. Enzymatic conversion processes may be used to treat the nucleic acids prior to sequencing, which can be performed in various ways. One example of a bisulfite-free conversion comprises a bisulfite-free and base-resolution sequencing method, TET-assisted pyridine borane sequencing (TAPS), for non-destructive and direct detection of 5-methylcytosine and 5-hydroxymethylcytosine without affecting unmodified cytosines. The methylation state of a CpG site in the corresponding plurality of CpG sites in the respective nucleic acid methylation fragment can be methylated when the CpG site is determined by the methylation sequencing to be methylated, and unmethylated when the CpG site is determined by the methylation sequencing to not be methylated.

A methylation sequencing assay (e.g., WGBS and/or targeted methylation sequencing) can have an average sequencing depth including but not limited to up to about 1,000×, 2,000×, 3,000×, 5,000×, 10,000×, 15,000×, 20,000×, or 30,000×. The methylation sequencing can have a sequencing depth that is greater than 30,000×, e.g., at least 40,000× or 50,000×. A whole-genome bisulfite sequencing method can have an average sequencing depth of between 20× and 50×, and a targeted methylation sequencing method has an average effective depth of between 100× and 1000×, where effective depth can be the equivalent whole-genome bisulfite sequencing coverage for obtaining the same number of sequence reads obtained by targeted methylation sequencing.

For further details regarding methylation sequencing (e.g., WGBS and/or targeted methylation sequencing), see, e.g., U.S. patent application Ser. No. 16/352,602, entitled “Methylation Fragment Anomaly Detection,” filed Mar. 13, 2019, and U.S. patent application Ser. No. 16/719,902, entitled “Systems and Methods for Estimating Cell Source Fractions Using Methylation Information,” filed Dec. 18, 2019, each of which is hereby incorporated by reference. Other methods for methylation sequencing, including those disclosed herein and/or any modifications, substitutions, or combinations thereof, can be used to obtain fragment methylation patterns. A methylation sequencing can be used to identify one or more methylation state vectors, as described, for example, in U.S. patent application Ser. No. 16/352,602, entitled “Anomalous Fragment Detection and Classification,” filed Mar. 13, 2019, or in accordance with any of the techniques disclosed in U.S. patent application Ser. No. 15/931,022, entitled “Model-Based Featurization and Classification,” filed May 13, 2020, each of which is hereby incorporated by reference.

The methylation sequencing of nucleic acids and the resulting one or more methylation state vectors can be used to obtain a plurality of nucleic acid methylation fragments. Each corresponding plurality of nucleic acid methylation fragments (e.g., for each respective genotypic dataset) can comprise more than 100 nucleic acid methylation fragments. An average number of nucleic acid methylation fragments across each corresponding plurality of nucleic acid methylation fragments can comprise 1000 or more nucleic acid methylation fragments, 5000 or more nucleic acid methylation fragments, 10,000 or more nucleic acid methylation fragments, 20,000 or more nucleic acid methylation fragments, or 30,000 or more nucleic acid methylation fragments. An average number of nucleic acid methylation fragments across each corresponding plurality of nucleic acid methylation fragments can be between 10,000 nucleic acid methylation fragments and 50,000 nucleic acid methylation fragments. The corresponding plurality of nucleic acid methylation fragments can comprise one thousand or more, ten thousand or more, 100 thousand or more, one million or more, ten million or more, 100 million or more, 500 million or more, one billion or more, two billion or more, three billion or more, four billion or more, five billion or more, six billion or more, seven billion or more, eight billion or more, nine billion or more, or 10 billion or more nucleic acid methylation fragments. An average length of a corresponding plurality of nucleic acid methylation fragments can be between 140 and 480 nucleotides.

Further details regarding methods for sequencing nucleic acids and methylation sequencing data are disclosed in U.S. patent application Ser. No. 17/191,914, titled “Systems and Methods for Cancer Condition Determination Using Autoencoders,” filed Mar. 4, 2021, which is hereby incorporated herein by reference in its entirety.

### III. WBC Contamination Detection

The analytics system implements WBC contamination detection to detect WBC contamination in samples used in the cancer classification workflow. In particular, the analytics system aims to prevent samples contaminated with WBC-shed DNA from downstream analyses, as the WBC-shed DNA can confound cancer detection when analyzed in conjunction with the cfDNA in the plasma, which may lead to false positive calls of cancer. In WBC contamination detection, the analytics system may determine whether a sample is contaminated by the WBC-shed DNA and may further determine a level of contamination. Such screening ensures training data used to train downstream models will not be skewed due to the WBC contamination. The screening also can reduce false positive predictions by withholding contaminated samples from further analyses. Upon detection of a contaminated sample, further remedial measures may be taken to address the contamination. Remedying sources of contamination thereby improves the physical assaying process and the sample processing.

Three different methodologies of WBC contamination detection are described herein this disclosure. A first methodology termed “Coverage-Based WBC Contamination Detection” aims to evaluate whether a sample has aberrant coverage at a set of genomic loci, which indicates WBC contamination. A second methodology termed “Methylation-Based WBC Contamination Detection” aims to deconvolve tissue type fractions based on methylation features derived from the sequence reads in a sample. Based on the deconvolved tissue type fractions, the analytic system may determine a distribution of tissue type fractions from a set of purified cfDNA samples to assess a likelihood that a test sample is contaminated. The purified cfDNA samples are purified to remove any non-cfDNA fragment, e.g., including WBC-derived fragments. A third methodology termed “Quantitative Coverage-Based WBC Contamination Detection” aims to evaluate an amount of WBC-shed DNA present in a sample based on maximizing a likelihood of observing coverages over a feature set of genomic loci. The principles described across these methodologies may be variably combined to form hybrid methodologies of assessing WBC contamination.

Each sample includes the sequence reads as derived from sequencing of DNA fragments contained in each physically collected biological sample. The sequence reads generally represent the amino acid sequences of DNA fragments, e.g., in the biological sample. The sequence reads may include methylation information, e.g., as described in FIGS. 2A & 2B. In some embodiments, the sequence reads may be derived from whole-genome bisulfite sequencing (WGBS) and/or targeted sequencing. In WGBS, there are sequence reads for each DNA fragment (or DNA molecule) contained in the biological sample. In WGBS, the sequence reads map to targeted genomic regions (or genomic loci) as pulled down from targeting probes. The sequence reads may otherwise include other types of sequencing information, e.g., single nucleotide polymorphisms, insertions or deletions, single tandem repeats, etc.

### III.A. Coverage-Based WBC Contamination Detection

FIGS. 3A-3C include flowcharts describing the first methodology of Coverage-Based WBC Contamination Detection. In particular, FIG. 3A illustrates a flowchart describing the identification of features for Coverage-Based WBC Contamination Detection, according to one or more embodiments. FIG. 3B illustrates a flowchart describing the training of a contamination model to predict WBC contamination based on coverage over a feature set of genomic loci, according to one or more embodiments. FIG. 3C illustrates a flowchart describing prediction of WBC contamination with the contamination model, according to one or more embodiments. The analytics system is described as performing each of the processes described in the flowcharts. Nonetheless, in other embodiments, different computing systems may perform each process.

FIG. 3A illustrates a flowchart describing the feature identification 300 for Coverage-Based WBC Contamination Detection, according to one or more embodiments.

The analytics system obtains 305 a first set of cfDNA samples from a first cohort of subjects and a second set of WBC samples from the first cohort of subjects. For example, each subject in the first cohort has provided at least one cfDNA sample and at least one WBC sample. A cfDNA sample includes sequence reads pertaining to cfDNA fragments. A WBC sample includes sequence reads pertaining to WBC-shed DNA fragments.

The analytics system determines 310, for each sample, a coverage of sequence reads overlapping each genomic locus in an initial set of genomic loci. The initial set of genomic loci may include up to all the genomic regions, e.g., all the targeted genomic regions in a targeted sequencing embodiment or all genomic regions in a WGBS embodiment. The coverage may be counted by identifying unique sequence reads that overlap partially, substantially, or wholly the genomic region. In some embodiments, a genomic region includes at least one CpG site. In other embodiments, a genomic region spans a set of CpG sites. In yet other embodiments, the genomic region is at least 50 bp, 100 bp, 150 bp, 200 bp, or 250 bp in length. Of the initial set of genomic regions, the analytics system may prune genomic regions that have variable coverage in cfDNA across samples. The analytics system may assess statistical variance in coverage across cfDNA samples to determine whether a particular genomic region has too much variability, e.g., above a statistical variance or spread in the distribution. The analytics system may further assess whether coverage is normally distributed, e.g., pruning or removing genomic regions with non-normal distributions.

As a working example, for Subject A's cfDNA sample, the analytics system determines a coverage for Genomic Locus 1 based on sequence reads in the cfDNA sample, a coverage for Genomic Locus 2 based on sequence reads in the cfDNA sample, and so on through the remaining genomic loci in the initial set. Likewise, for Subject A's WBC sample, the analytics system determines a coverage for Genomic Locus 1 based on sequence reads in the WBC sample, a coverage for Genomic Locus 2 based on sequence reads in the WBC sample, and so on through the remaining genomic loci in the initial set.

The analytics system may further normalize the coverages for each sample. For example, the analytics system determines a sequencing depth based on the sequence reads in the sample. With the sequencing depth, the analytics system may normalize each coverage by dividing by the sequencing depth. Normalizing the coverage ensures differential sequencing depth across samples does not skew analyses.

The analytics system determines 315, for each genomic locus, a ratio of coverage between the WBC samples and the cfDNA samples. The analytics system may, for each subject, calculate a ratio of coverage for each genomic locus between the WBC sample and the cfDNA sample. Following the example above, for Subject A, the analytics system may calculate a ratio of coverage for Genomic Locus 1 by dividing the coverage at Genomic Locus 1 from the WBC sample by the coverage at Genomic Locus 1 from the cfDNA sample, and so on with remaining genomic loci in the initial set. With each subject having its own ratios of coverage across the genomic loci, the analytics system may average the ratios of coverage to determine the aggregate ratio of coverage between the WBC samples and the cfDNA samples.

The analytics system determines 320 a feature set of genomic loci with ratios of coverage above a threshold for use in assessing WBC contamination. A genomic locus in the feature set may also be termed a “WBC contamination marker” (or “contamination marker,” more generally). The analytics system determines the feature set utilizing the threshold ratio. The analytics system may further rank the genomic loci based on the ratios of coverage and select a top number from the ranking.

FIG. 3B illustrates a flowchart describing the training 330 of a contamination model to predict WBC contamination based on coverage over a feature set of genomic loci, according to one or more embodiments.

The analytic system obtains 335 a set of cfDNA samples from non-cancer subjects including a first subset and a second subset. The cfDNA samples may be purified to ensure there's no confounding WBC-shed DNA signal, and may further be purified to ensure there's no hematological disease signal. The set of cfDNA samples from the non-cancer subjects may be split into a first and a second subset. The first subset may be used to train the contamination model to predict a contamination metric for samples with the second subset used to determine a contamination threshold for calling WBC contamination from the contamination metric. In some embodiments, the subsets can be overlapping, or the same.

The analytics system determines 340, for each sample in the first subset and the second subset, a coverage of sequence reads overlapping each genomic locus in the feature set. The coverage may be counted by identifying unique sequence reads that overlap partially, substantially, or wholly the genomic region. The coverages may be further normalized, e.g., as according to sequencing depth of the sample.

The analytics system generates 345, for each genomic locus in the feature set, a distribution of coverage based on the coverages of the samples of the first subset. A distribution for each genomic locus can be described by its mean and standard deviation. The distribution is useful in determining the likelihood of observing a coverage at the genomic locus in a test sample. Upon generation, there is a distribution of coverage for each genomic locus based on the samples from the non-cancer subjects.

The analytics system determines 350, for each sample of the second subset, a statistical likelihood of observing the coverage at each genomic locus based on the distribution of coverage for the genomic locus. For example, with Sample B of the second subset, the analytics system determines a statistical likelihood of observing the coverage at Genomic Locus 1 based on the distribution of coverage for Genomic Locus 1. In some embodiments, the statistical likelihood of a coverage is a z-score based on the distribution of coverage. The z-score indicates a number of standard deviations away from the mean of the distribution. In other embodiments, the statistical likelihood of a coverage is a p-value based on the distribution of coverage, indicating a likelihood of observing the coverage of the sample at that genomic locus or greater. The p-value is determined by the proportion of the distribution of coverage greater than the coverage of the sample.

The analytics system generates 355, for each sample of the second subset, a contamination metric by combining the statistical likelihoods across the genomic loci in the feature set. In embodiments with the z-score, the analytics system may sum the z-scores across the genomic loci in the feature set to generate the contamination metric for the sample. In some embodiments, the analytics system may sum the absolute value of z-scores. In some embodiments, the analytics system may sum the absolute value of z-scores above a threshold value to generate the contamination metric. In embodiments with the p-value score, the analytics system may generate the contamination metric as a truncated p-value product of the p-values across the genomic loci. The truncated p-value product may be calculated by combining p-values below some specified cut-off value.

The analytics system determines 360 a distribution of contamination metric for the second subset of samples. The analytics system may generate the distribution with the contamination metrics calculated for the second subset of samples. The distribution can be represented by a mean and standard deviation.

The analytics system determines 365 a contamination threshold based on the distribution of contamination metric. As noted, the second subset of samples are derived from non-cancer subjects (e.g., otherwise healthy subjects). The distribution thus represents the normal distribution of non-contaminated samples from non-cancer subjects. With the distribution, the analytics system can set a specificity threshold, e.g., 95%, 96%, 97%, 98%, 99%, 99.5%, etc. Based on this specificity threshold, the analytics system can determine a contamination threshold for use in calling whether there is WBC contamination. The contamination threshold splits the distribution such that having a contamination metric above the contamination threshold indicates that it is highly unlikely to naturally observe such a contamination metric, such that it is likely that the sample is WBC contaminated. In other words, the proportion of the distribution below the contamination threshold equals the specificity threshold. A contamination model may be generated to input the coverages over the feature set of genomic loci for a sample and determine whether the sample is WBC contaminated.

The analytics system may further evaluate the predictability of the feature set of the contamination model against a null distribution of other candidate sets. The analytics system may identify genomic loci from the initial set with equal coverage between cfDNA samples and WBC samples. From that set of genomic loci with equal coverage, the analytics system may randomly select candidate sets to compare predictability. Each candidate set of genomic loci may be of the same size as the feature set. For example, if the feature set contains 50 genomic loci (or contamination markers), then the candidate sets may also contain 50 genomic loci (or contamination markers). The analytic system may similarly train alternative contamination models according to the model training 330. With the trained contamination model trained with the feature set and the alternative contamination models, the analytic system may use a validation set of samples including samples with known WBC contamination (e.g., in silico or titrated from WBC samples). The analytics system can evaluate how many of the contaminated samples are correctly identified by the contamination model trained with the feature set and the alternative contamination models.

FIG. 3C illustrates a flowchart describing WBC contamination prediction 370 with the contamination model, according to one or more embodiments. In one or more embodiments, the contamination model includes the various distributions generated in the model training 330 of FIG. 3B. For example, the contamination model includes the distributions of coverage and the distribution of contamination metric. The contamination model may further include the contamination threshold for calling WBC contamination in a sample.

The analytics system obtains 375 a test sample from a test subject. The test sample may include sequence reads sequenced from a biological sample collected from the test subject. The test sample may be used in validation of the contamination model, or may be from a test subject to undergo cancer classification. The biological sample may be a blood sample that is processed to isolate plasma from the WBC buffy coat and the solid mass including red blood cell and other particulates in the blood. The sequence reads may correspond to the cfDNA fragments in the plasma. The analytics system applies the contamination model (e.g., as trained by the model training 330 of FIG. 3B) to the test sample to assess whether the sample has WBC contamination.

The analytics system determines 380 a coverage of sequence reads overlapping each genomic locus in the feature set. The coverage may be counted by identifying unique sequence reads that overlap partially, substantially, or wholly the genomic region. The coverages may be further normalized, e.g., as according to sequencing depth of the sample.

The analytics system determines 385 a statistical likelihood of observing the coverage at each genomic locus based on the distribution of coverage for the genomic locus. In some embodiments, the statistical likelihood of a coverage is a z-score based on the distribution of coverage. In other embodiments, the statistical likelihood of a coverage is a p-value based on the distribution of coverage, indicating a likelihood of observing the coverage of the sample at that genomic locus or greater.

The analytics system generates 390 a contamination metric for the test sample by combining the statistical likelihoods across the genomic loci. In embodiments with the z-score, the analytics system may sum the z-scores across the genomic loci in the feature set to generate the contamination metric for the sample. In some embodiments, the analytics system may sum the absolute value of z-scores. In some embodiments, the analytics system may sum the absolute value of z-scores above a threshold value to generate the contamination metric. In embodiments with the p-value score, the analytics system may generate the contamination metric as a truncated p-value product of the p-values across the genomic loci. The truncated p-value product may be calculated by combining p-values below some specified cut-off value.

The analytics system determines 395 whether the test sample has WBC contamination if the contamination metric for the test sample is at or above the contamination threshold. The contamination threshold is a value identified based on the distribution of contamination metric that ensures some level of specificity, which avoids false positive calling of WBC contamination.

Upon determining a test sample is WBC contaminated, the analytics system may implement remedial measures. For one, the analytics system may provide a notification to a healthcare provider indicating the detection of WBC contamination in the test sample. The notification may further recommend that the healthcare provider collects a subsequent biological sample for sequencing and analysis in the cancer classification workflow. The notification can further inform whether particular clinical supplies, particular clinicians, or sample processing techniques are more likely to result in WBC contamination. For two, the analytics system may withhold the test sample from further analyses in the cancer classification workflow to avoid false positive calls of cancer from confounding WBC-shed DNA. Withholding the sample from downstream analyses may entail filtering training samples, improving the training of the cancer classification models.

### III.B. Methylation-Based WBC Contamination Detection

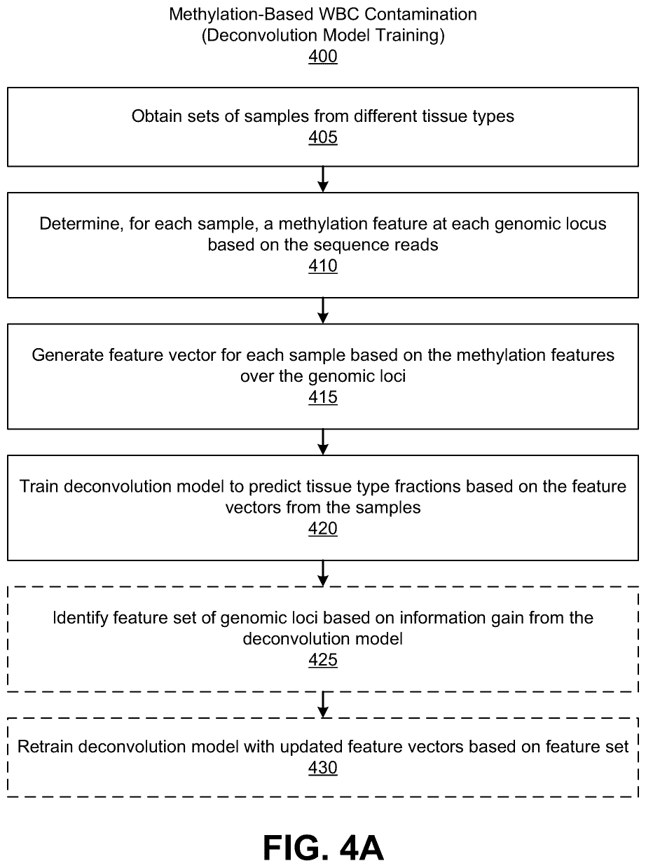

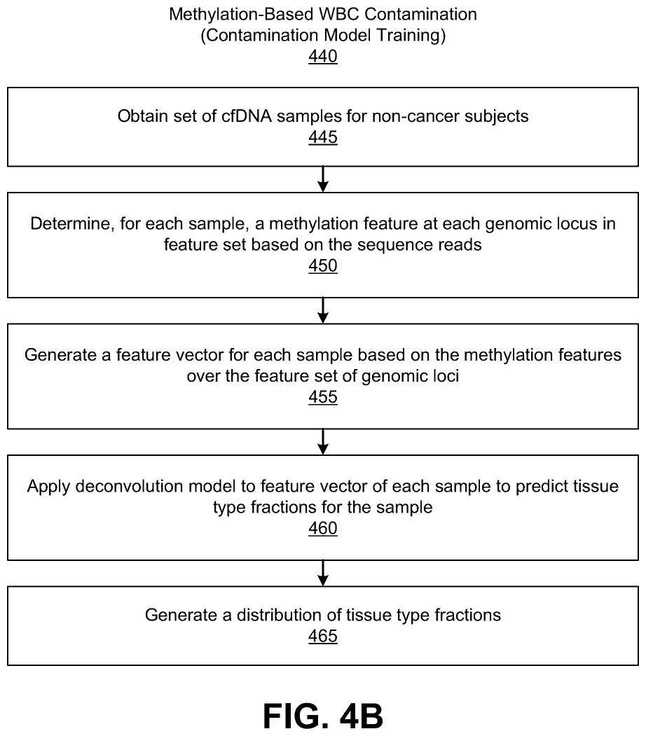

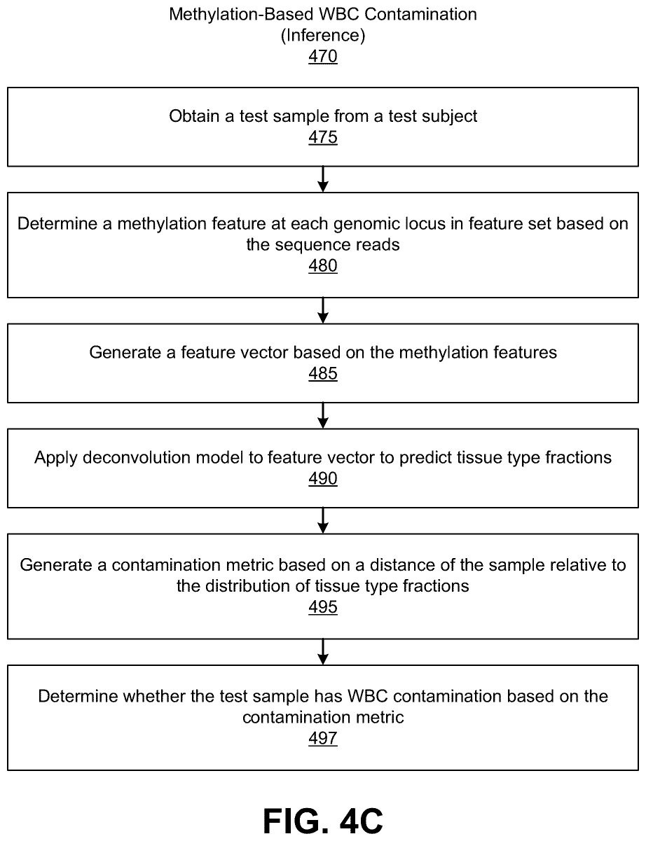

FIGS. 4A-4C include flowcharts describing the second methodology of Methylation-Based WBC Contamination Detection. In particular, FIG. 4A illustrates a flowchart describing the training 400 of a deconvolution model for Methylation-Based WBC Contamination Detection, according to one or more embodiments. FIG. 4B illustrates a flowchart describing the training 440 of a contamination model to predict WBC contamination based on predicted tissue type fractions, according to one or more embodiments. FIG. 4C illustrates a flowchart describing WBC contamination prediction 470 with the contamination model, according to one or more embodiments. The analytics system is described as performing each of the processes described in the flowcharts. Nonetheless, in other embodiments, different computing systems may perform each process.

FIG. 4A illustrates a flowchart describing the training 400 of a deconvolution model for Methylation-Based WBC Contamination Detection, according to one or more embodiments. The deconvolution model may be trained to input methylation information and to output tissue type fractions.

The analytics system obtains 405 sets of samples from different tissue types. Each set of samples may be labeled by a tissue type of the different tissue types. In some embodiments, a set of samples may be further segregated into subsets based on tissue sub-types. The samples of one tissue type are generally derived from biological samples with DNA fragments derived from cells of the tissue type. For example, the biological samples may be biopsy samples taken from particular tissues. Tissue types may also include cell types.

In some embodiments, the set of tissue types is selected as a combination of the following tissue types: erythrocyte progenitor cell type, megakaryocyte cell type, monocyte cell type, granulocyte cell type, lymphocyte cell type, epithelial cell type, vascular endothelial cell type, hepatocyte cell type, adipocyte cell type, endocrine cell type, and muscle cell type. In other embodiments, the set of tissue types is selected as a combination of the following tissue types: myeloid circulating tissue type (e.g., which may be inclusive of the erythrocyte progenitor cell type and the megakaryocyte cell type), myeloid non-circulating tissue type (e.g., which may be inclusive of the monocyte cell type and the granulocyte cell type), lymphocyte cell type, epithelial cell type, vascular endothelial cell type, and hepatocyte cell type.

The analytics system determines 410, for each sample, a methylation feature at each genomic locus based on the sequence reads. The genomic locus may include one or more methylation sites, e.g., CpG sites. The genomic loci may be an initial set of genomic loci, e.g., up to all genomic loci covered by the sequencing process. The sequence reads may further include methylation information, e.g., as determined by FIGS. 2A & 2B. A methylation feature characterizes the methylation status of one or more sequence reads overlapping the genomic locus. In some embodiments, the analytics system determines multiple methylation features at one or more of the genomic loci. In one example, the methylation feature may be a methylation density at the genomic locus. In another example, the methylation feature may be a count (or a proportion) of sequence reads overlapping the genomic locus that are highly methylated or highly unmethylated. In a third example, the methylation feature may be a count (or a proportion) of sequence reads having a particular methylation variant at the genomic locus.

The analytics system generates 415 a feature vector for each sample based on the methylation features over the genomic loci. The feature vector represents the methylation features as determined in step 410. For example, the feature vector may list all the methylation features as elements. In other examples, the analytics system may combine various methylation features to reduce dimensionality of the feature vector.

The analytics system trains 420 the deconvolution model to predict tissue type fractions based on the feature vectors from the samples. The deconvolution model may be a machine-learning model. The deconvolution model is generally trained to input the feature vectors of samples to predict the tissue type of the sample. Based on the accuracy of the prediction, the analytics system may adjust weights or other parameters in the deconvolution model to improve the accuracy of the prediction. In some embodiments, the analytics system may train the deconvolution model in multiple folds. In each fold, the analytics system adjusts weights and/or parameters in the deconvolution model using a subset of the training samples. The trained deconvolution model can input a feature vector representing the methylation information of the sequence reads and output a prediction of tissue type fractions. Each tissue type fraction is a fractional contribution of a tissue type to the DNA fragments (or sequence reads) present in the sample. For example, for a set of six tissue types, the deconvolution model is configured to output six tissue type fractions, one tissue type fraction for each tissue type. The sum of the tissue type fractions can equate to 100% or 1. The analytics system may further refine and optimize the deconvolution model.

The analytics system may identify 425 a feature set of genomic loci based on information gain from the deconvolution model. The analytics system may assess information gain by assessing the entropy of each feature (or methylation feature) in the feature vectors. For example, mutual information can provide a measure of the mutual dependence between two conditions of interest (e.g., tissue types) sampled simultaneously. A mutual information score can denote the discriminative power of a methylation feature for discriminating between the two tissue types. Information gain can be assessed during training of the deconvolution model, e.g., after each epoch of training. The analytics system can rank the features based on the information gain. The analytics system may identify the feature set as features having information gain above an information gain threshold. In other embodiments, the analytics system may identify the feature set as the top number of features ranked based on information gain. The analytics system may select the feature set based on other selection criteria. For example, the analytics system may identify features that are sufficiently spread throughout the genome. As another example, the analytics system may evaluate whether features are stable, e.g., whether the features are normally distributed in the overall population. The features set may indicate one or more methylation features at one or more genomic loci.

The analytics system may retrain 430 the deconvolution model with updated feature vectors based on the identified feature set. The analytics system may update feature vectors, e.g., retaining features in the feature set. For example, the feature selection may yield a set of genomic loci that are most discriminative in predicting tissue types, and the analytic system may update the feature vectors for the samples to only retain methylation feature(s) pertaining to that feature set of genomic loci. The analytics system may then retrain the deconvolution model using the updated feature vectors. The analytics system may further use a holdout set of samples to assess predictive accuracy of the deconvolution model.

FIG. 4B illustrates a flowchart describing the training 440 of a contamination model to predict WBC contamination based on predicted tissue type fractions, according to one or more embodiments.

The analytics system obtains 445 a set of cfDNA samples from non-cancer subjects. Each cfDNA sample may be purified to ensure DNA fragments and corresponding sequence reads are not contaminated with WBC-shed DNA.

The analytics system determines 450, for each sample, a methylation feature at each genomic locus in the feature set based on the sequence reads. The analytics system may determine multiple features per genomic locus. The feature set of genomic loci may be determined via assessing information gain, e.g., as described in FIG. 4A.

The analytics system generates 455 a feature vector for each sample based on the methylation features over the feature set of genomic loci. The feature vector may include the methylation features over the feature set of genomic loci, or may include some combined representation of the methylation features.

The analytics system applies 460 the deconvolution model to the feature vector of each sample to predict the tissue type fractions for the sample. The deconvolution model may be trained according to the training 400 described in FIG. 4A. Each tissue type fraction is a predicted fractional contribution of the tissue type in sourcing the DNA fragments (or sequence reads thereof) in the sample. The tissue type fractions may be represented in a vector.

The analytics system generates 465 a distribution of tissue type fractions for the samples. The distribution is a multivariate distribution in multivariate vector space. The number of variables equals the number of tissue types predicted. The distribution is formed by the predicted tissue type fractions of the samples. The distribution may be represented by a mean tissue type fractions as an average of tissue type fractions across the samples and may further be represented by principal axes in the vector space. The distribution represents a null distribution of cfDNA samples from the non-cancer population. The contamination model implements the distribution to calculate a p-value representing the likelihood of observing a predicted tissue type fraction for a test sample in the null distribution. Anything but a very low likelihood would be deemed within expectation of a cfDNA sample without WBC contamination. A very low likelihood would be deemed an outlier to the distribution and indicative of WBC contamination.

FIG. 4C illustrates a flowchart describing WBC contamination prediction 470 with the contamination model, according to one or more embodiments.

The analytics system obtains 475 a test sample from a test subject. The test sample may be used in validation of the contamination model, or may be from a test subject to undergo cancer classification. The test sample comprises sequence reads pertaining to DNA fragments in the biological sample collected from the test subject. In one or more embodiments, the test sample is to undergo cancer classification to determine whether the cfDNA fragments in the biological sample indicate presence or likelihood of presence of cancer. The analytics system applies the contamination model to predict whether the test sample is WBC contaminated to prevent confounding the cancer classification with WBC-shed DNA.

The analytics system determines 480 a methylation feature at each genomic locus in the feature set based on the sequence reads. The methylation features may be, for example, methylation density, count or proportion of sequence reads that are highly methylated, count or proportion of sequence reads that are highly unmethylated, count or proportion of sequence reads that have a particular methylation variant, etc.

The analytics system generates 485 a feature vector for the test sample based on the methylation features. The feature vector may list all the methylation features or may combine one or more of the methylation features.

The analytics system applies 490 the deconvolution model to the feature vector to predict tissue type fractions for the test sample. The tissue type fractions indicate fractional contribution of a plurality of tissue types sourcing the DNA fragments (and corresponding sequence reads) in the biological sample.

The analytics system generates 495 a contamination metric for the test sample based on a distance of the sample relative to the distribution of the tissue type fractions. In one or more embodiments, the distance may be calculated based on the mean tissue type fractions and one or more principal components describing the distribution of tissue type fractions. In one or more embodiments, the distance is a Mahalanobis distance:

$$Mahalanobis Distance = \sqrt{{( x - μ )}^{T} {\sum }^{- 1} ( x - μ )} \qquad ( 1 )$$

wherein x represents the tissue type fractions for a sample, μ represents the mean tissue type fractions of the null distribution, and Σ represents a positive-definite covariance matrix. The contamination metric may be represented as:

$$f ( x ) = \frac{exp exp ( - \frac{1}{2} {( x - μ )}^{T} {\sum }^{- 1} ( x - μ ) )}{\sqrt{{( 2 π )}^{k} \vert \sum \vert}} \qquad ( 2 )$$

which leverages the Mahalanobis distance.

The analytics system determines 497 whether the test sample has WBC contamination based on the contamination metric. In one or more embodiments, the contamination metric is a p-value indicating a likelihood of observing the predicted tissue type fractions in cfDNA samples of a non-cancer population. In such embodiments, the p-value is compared against a p-value threshold. If the p-value is below the p-value threshold, then the analytics system calls the test sample to be WBC contaminated. If the p-value is above the p-value threshold, then the analytics system calls the test sample to not be WBC contaminated.

Upon determining a test sample is WBC contaminated, the analytics system may implement remedial measures. For one, the analytics system may provide a notification to a healthcare provider indicating the detection of WBC contamination in the test sample. The notification may further recommend that the healthcare provider collects a subsequent biological sample for sequencing and analysis in the cancer classification workflow. The notification can further inform whether particular clinical supplies, particular clinicians, or sample processing techniques are more likely to result in WBC contamination. For two, the analytics system may withhold the test sample from further analyses in the cancer classification workflow to avoid false positive calls of cancer from confounding WBC-shed DNA. Withholding the sample from downstream analyses may entail filtering training samples, improving the training of the cancer classification models.

### III.C. Quantitative Coverage-Based WBC Contamination Detection

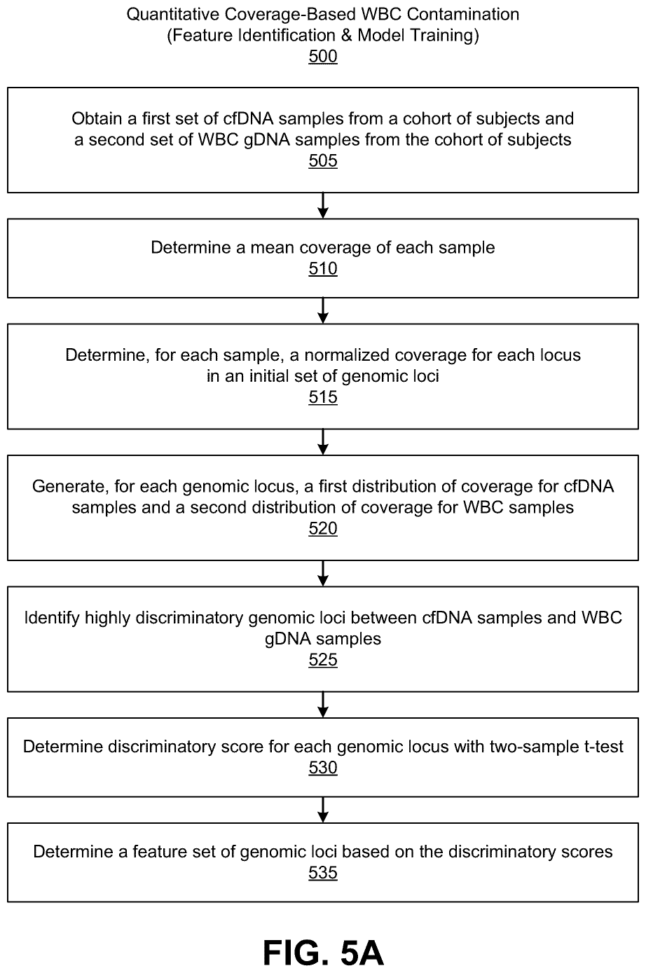

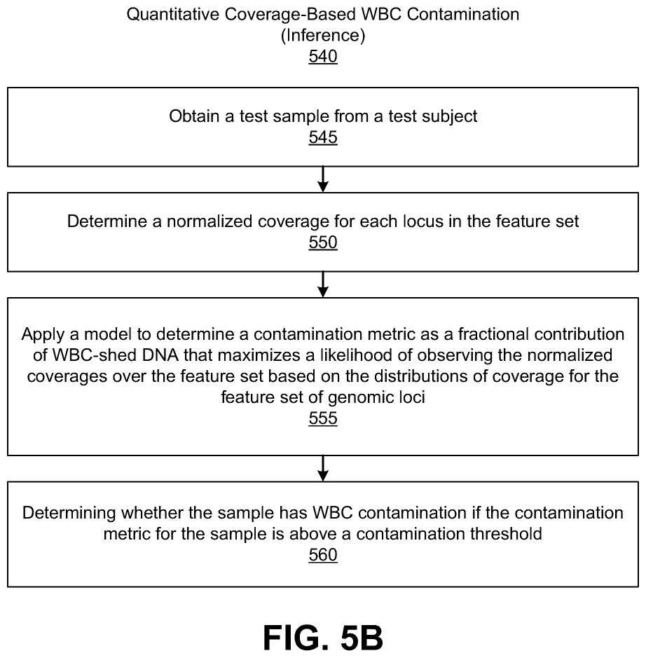

FIGS. 5A & 5B include flowcharts describing the third methodology of Quantitative Coverage-Based WBC Contamination Detection. In particular, FIG. 5A illustrates a flowchart describing the feature identification and training 500 of a contamination model for Quantitative Coverage-Based WBC Contamination Detection, according to one or more embodiments. FIG. 5B illustrates a flowchart describing WBC contamination prediction 540 with the contamination model, according to one or more embodiments. The analytics system is described as performing each of the processes described in the flowcharts. Nonetheless, in other embodiments, different computing systems may perform each process.

FIG. 5A illustrates a flowchart describing the feature identification and training 500 of a contamination model for Quantitative Coverage-Based WBC Contamination Detection, according to one or more embodiments.

The analytics system obtains 505 a first set of cfDNA samples from a cohort of subjects and a second set of WBC gDNA samples from the cohort of subjects. The cfDNA samples may be purified to ensure only cfDNA fragments compose the biological sample from which the sequence reads are derived. The WBC gDNA samples may be purified to ensure only WBC genomic DNA fragments compose the biological sample from which the sequence reads are derived. Each subject in the cohort may have provided at least one cfDNA sample and at least one WBC gDNA sample.

The analytics system determines 510 a mean coverage of each sample. In some embodiments, the analytics system determines the mean coverage over all genomic loci or markers sequenced for. The genomic loci may include binary markers and semibinary markers. A binary marker assesses whether sequence reads are one of two states at a genomic locus. A semibinary marker assesses whether there are sequence reads that cover one of plurality of states at a genomic locus. In some embodiments, the analytics system determines the mean coverage as an average over the binary markers.

The analytics system determines 515, for each sample, a normalized coverage for each locus (or marker) in an initial set of genomic loci. Each sample is separately normalized by its mean coverage. For example, with Sample C, the analytics system may normalize all coverages across the genomic loci by the mean coverage, yielding normalized coverages. To further explain, at Genomic Locus 1, the analytics system calculates a normalized coverage for Sample C by dividing the coverage at Genomic Locus 1 by the mean coverage of Sample C.

The analytics system generates 520, for each genomic locus, a first distribution of coverage for cfDNA samples and a second distribution of coverage for WBC gDNA samples. The analytics system may calculate various characteristics or statistics of the distributions. For example, the analytics system may assess normality of the distributions. The analytics system may further define the distributions based on mean and standard deviation.

The analytics system identifies 525 highly discriminatory genomic loci between cfDNA samples and WBC gDNA samples. The analytics system may assess the distance between the two distributions at each genomic locus. The distance may be calculated as the area under the curve for binary classification and/or as a Hodges-Lehmann estimator of difference. The analytics system may identify genomic loci to be highly discriminatory if the area under the curve is >0.99 or <0.01. Other thresholds may be used, e.g., >0.98 or <0.02; >0.95 or <0.05, etc. The analytics system may further assess whether there is at least some threshold difference in mean normalized coverage between the two distributions, e.g., >0.5.

The analytics system determines 530 a discriminatory score for each genomic locus with a two-sample t-test. The two-sample t-test evaluates the distinctiveness of pairwise samples for each subject. The discriminatory score can be corrected with Bonferroni correction. The discriminatory score may further be based on a difference in means between the two distributions. The analytics system may further assess goodness-of-fit of normality, i.e., whether the two distributions are sufficiently normal (Gaussian).

The analytics system determines 535 the feature set of genomic loci based on the discriminatory scores. The analytics system may rank the genomic loci based on discriminatory score. The analytics system may select genomic loci to form the feature set with discriminatory scores above a threshold. In other embodiments, the analytics system may select the top number of genomic loci ranked according to discriminatory scores to form the feature set. The top number may be 5, 10, 15, 20, 25, 30, 35, 40, 45, 50, etc. The top number may be chosen based on comparative validation tests on predictive accuracy of each candidate set. The contamination model includes the paired distributions of coverage from cfDNA samples and WBC gDNA samples over the feature set of genomic loci. For example, with 50 genomic loci in the feature set, the contamination model includes the first distribution of coverage for cfDNA samples and the second distribution of coverage for WBC gDNA samples for each of the 50 genomic loci, yielding a total of 50 distributions of coverage for cfDNA samples and 50 distributions of coverage for WBC gDNA samples.

In one or more embodiments, the feature set of genomic loci includes sequences annotated with respect to its genomic location, as described in Table 1. Based on this feature set of genomic loci, a panel of probes may target such genomic loci.

TABLE 1
Feature Set of Genomic Loci for Quantitative
Coverage-Based WBC Contamination Detection

| Genomic Locus | Chromosome | Start Position | End Position |
|---|---|---|---|
| NO. 1 | chr1 | 48463405 | 48463465 |
| NO. 2 | chr1 | 114302509 | 114302713 |
| NO. 3 | chr1 | 173991621 | 173991652 |
| NO. 4 | chr1 | 173991673 | 173991703 |
| NO. 5 | chr1 | 186344716 | 186344732 |
| NO. 6 | chr2 | 24306893 | 24306923 |
| NO. 7 | chr2 | 61990959 | 61991302 |
| NO. 8 | chr2 | 65086545 | 65086575 |
| NO. 9 | chr3 | 32612638 | 32612776 |
| NO. 10 | chr3 | 44802847 | 44803653 |
| NO. 11 | chr3 | 169482521 | 169482762 |
| NO. 12 | chr3 | 182510951 | 182511159 |
| NO. 13 | chr3 | 193721032 | 193721474 |
| NO. 14 | chr4 | 6717791 | 6717914 |
| NO. 15 | chr4 | 37688563 | 37688650 |
| NO. 16 | chr5 | 79551297 | 79551443 |
| NO. 17 | chr5 | 139726429 | 139726535 |
| NO. 18 | chr5 | 148724797 | 148724903 |
| NO. 19 | chr5 | 158635121 | 158635542 |
| NO. 20 | chr6 | 143266116 | 143266222 |
| NO. 21 | chr7 | 77167772 | 77167877 |
| NO. 22 | chr7 | 105752146 | 105752177 |
| NO. 23 | chr7 | 140098227 | 140098408 |
| NO. 24 | chr8 | 28258794 | 28258873 |
| NO. 25 | chr9 | 73028898 | 73029038 |
| NO. 26 | chr9 | 98189304 | 98189365 |
| NO. 27 | chr10 | 92617903 | 92618084 |
| NO. 28 | chr11 | 18720142 | 18720172 |
| NO. 29 | chr11 | 46264957 | 46265020 |
| NO. 30 | chr11 | 62192261 | 62192426 |
| NO. 31 | chr11 | 66079388 | 66079967 |
| NO. 32 | chr11 | 95522931 | 95522961 |
| NO. 33 | chr11 | 128391754 | 128392753 |
| NO. 34 | chr11 | 130083120 | 130083174 |
| NO. 35 | chr12 | 12877457 | 12877617 |
| NO. 36 | chr12 | 59989993 | 59990174 |
| NO. 37 | chr12 | 69080590 | 69080655 |
| NO. 38 | chr13 | 41345256 | 41345437 |
| NO. 39 | chr13 | 52585268 | 52585560 |
| NO. 40 | chr14 | 62161812 | 62161901 |
| NO. 41 | chr14 | 74111441 | 74111579 |
| NO. 42 | chr14 | 105781804 | 105781910 |
| NO. 43 | chr15 | 40074970 | 40075102 |
| NO. 44 | chr15 | 99191330 | 99191958 |
| NO. 45 | chr15 | 99192161 | 99192732 |
| NO. 46 | chr16 | 3156726 | 3156757 |
| NO. 47 | chr16 | 4852974 | 4853190 |
| NO. 48 | chr16 | 72699009 | 72699159 |
| NO. 49 | chr16 | 79804533 | 79804785 |
| NO. 50 | chr17 | 7199729 | 7199968 |
| NO. 51 | chr17 | 29421875 | 29422387 |
| NO. 52 | chr17 | 41277286 | 41277500 |
| NO. 53 | chr17 | 56429758 | 56429999 |
| NO. 54 | chr18 | 2571377 | 2571523 |
| NO. 55 | chr18 | 33077969 | 33078048 |
| NO. 56 | chr18 | 47792664 | 47792905 |
| NO. 57 | chr19 | 2273763 | 2274036 |
| NO. 58 | chr19 | 45908809 | 45908856 |
| NO. 59 | chr19 | 53030695 | 53031101 |
| NO. 60 | chr19 | 58859150 | 58859180 |
| NO. 61 | chr20 | 19997779 | 19997896 |
| NO. 62 | chr20 | 56784796 | 56785113 |
| NO. 63 | chr21 | 34777011 | 34777038 |
| NO. 64 | chr22 | 27068858 | 27069039 |

FIG. 5B illustrates a flowchart describing WBC contamination prediction 540 with the contamination model, according to one or more embodiments. In one or more embodiments, the contamination model includes the various distributions generated in the training 500 of FIG. 5A. For example, the contamination model includes the paired distributions of coverage across the feature set of genomic loci.

The analytics system obtains 545 a test sample from a test subject. The test sample may include sequence reads sequenced from a biological sample collected from the test subject. The test sample may be used in validation of the contamination model, or may be from a test subject to undergo cancer classification. The biological sample may be a blood sample that is processed to isolate plasma from the WBC buffy coat and the solid mass including red blood cell and other particulates in the blood. The sequence reads may correspond to the cfDNA fragments in the plasma. The analytics system applies the contamination model (e.g., as trained by the training 500 of FIG. 5A) to the test sample to assess whether the sample has WBC contamination.

The analytics system determines 550 a normalized coverage for each genomic locus in the feature set. The normalized coverage may be determined by first determining coverage at each genomic locus in the feature set, then normalizing the coverage by the mean coverage of the test sample. Normalization may alternatively be done according to sequencing depth.

The analytics system applies 555 the contamination model to determine a contamination metric as a fractional contribution of WBC gDNA that maximizes a likelihood of observing the normalized coverages over the feature set based on the distributions of coverage for the feature set of genomic loci. The contamination model may include a maximum likelihood function, e.g.:

$$Optimize π : {\sum }_{i = 1}^{1} log log ( N ( {x}_{i} \vert {μ}_{i} , {σ}_{i} ) ) \qquad ( 3 )$$

wherein i is any given genomic locus ranging from {1, 2, . . . , I}, π is the fractional contribution of WBC-shed DNA to the sample, and N is a combinative normal distribution of the paired distributions defined by μᵢ and σᵢ. Further:

$${μ}_{i} = π \cdot {}^{g} {μ}_{i} + ( 1 - π ) \cdot {}^{cf} {μ}_{i} \qquad ( 4 )$$

$${σ}_{i}^{2} = π \cdot {}^{g} {σ}_{i}^{2} + ( 1 - π ) \cdot {}^{cf} {σ}_{i}^{2} \qquad ( 5 )$$

wherein ^{g}μᵢ is the mean normalized coverage for target i in WBC gDNA, ^{cf}μᵢ is the mean normalized coverage for target i in cfDNA, ^{g}σᵢ is the standard deviation for target i in WBC gDNA, and ^{cf}σᵢ is the standard deviation for target i in cfDNA.

The analytics system determines 560 whether the test sample has WBC contamination if the contamination metric for the sample is above a contamination threshold. As the above contamination metric is calculated as a fractional contribution of gDNA to the test sample, the contamination threshold may be a tolerable amount of gDNA contribution. This contamination threshold may be set as 0.05, 0.04, 0.03, 0.02, etc.

Upon determining a test sample is WBC contaminated, the analytics system may implement remedial measures. For one, the analytics system may provide a notification to a healthcare provider indicating the detection of WBC contamination in the test sample. The notification may further recommend that the healthcare provider collects a subsequent biological sample for sequencing and analysis in the cancer classification workflow. The notification can further inform whether particular clinical supplies, particular clinicians, or sample processing techniques are more likely to result in WBC contamination. For two, the analytics system may withhold the test sample from further analyses in the cancer classification workflow to avoid false positive calls of cancer from confounding WBC-shed DNA. Withholding the sample from downstream analyses may entail filtering training samples, improving the training of the cancer classification models.

### IV. Cancer Classifier for Determining Cancer

Cancer classification involves extracting genetic features and applying one or more models to the extracted features to determine a cancer prediction. The analytics system aggregates extracted features into a feature vector which can then be input into a trained cancer prediction model to determine a cancer prediction based on the input feature vector. The cancer prediction may comprise one or more labels and/or one or more values. One label may be binary, indicating a presence or absence of cancer in the test subject. Another label may be multiclass, indicating one or more particular cancer types from a plurality of screened cancer types. One value may indicate a likelihood of presence of cancer. Another value may indicate a likelihood of absence of cancer. Yet another value may otherwise indicate another prognosis of the cancer. For example, the value may quantify a progression and/or an aggression of the cancer.

In one or more embodiments, the feature vectors input into the cancer classifier are based on set of informative fragments determined from the test sample.

In some embodiments, a cancer classifier may be a machine-learned model comprising a plurality of classification parameters and a function representing a relation between the feature vector as input and the cancer prediction as output. Inputting the feature vector into the function with the classification parameters yields the cancer prediction. The machine-learned model may be trained using training samples derived from individuals with known cancer diagnoses. The training samples may be divided into cohorts of varying labels. For example, there may be a cohort of training samples for each cancer type.

### IV.A. Identifying Informative Fragments

The analytics system can determine informative fragments for a sample using the sample's methylation state vectors. For each fragment in a sample, the analytics system can determine whether the fragment is an informative fragment using the methylation state vector corresponding to the fragment. In some embodiments, the analytics system calculates a p-value score for each methylation state vector describing a probability of observing that methylation state vector or other methylation state vectors even less probable in the healthy control group. The process for calculating a p-value score is further discussed below in Section IV.A.i. P-Value Filtering. The analytics system may determine fragments with a methylation state vector having below a threshold p-value score as informative fragments. In some embodiments, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining informative fragments. Examples of other probabilistic models include a mixture model, a deep probabilistic model, etc. In some embodiments, the analytics system may use any combination of the processes described below for identifying informative fragments. With the identified informative fragments, the analytics system may filter the set of methylation state vectors for a sample for use in other processes, e.g., for use in training and deploying a cancer classifier.

IV.A.i. P-Value Filtering

In some embodiments, the analytics system calculates a p-value score for each methylation state vector compared to methylation state vectors from fragments in a healthy control group. The p-value score can describe a probability of observing the methylation status matching that methylation state vector or other methylation state vectors even less probable in the healthy control group. In order to determine a DNA fragment to be informatively methylated, the analytics system can use a healthy control group with a majority of fragments that are normally methylated. When conducting this probabilistic analysis for determining informative fragments, the determination can hold weight in comparison with the group of control subjects that make up the healthy control group. To ensure robustness in the healthy control group, the analytics system may select some threshold number of healthy individuals to source samples including DNA fragments. FIG. 6A below describes the method of generating a data structure for a healthy control group with which the analytics system may calculate p-value scores. FIG. 6B describes the method of calculating a p-value score with the generated data structure.

FIG. 6A is a flowchart describing a process 600 of generating a data structure for a healthy control group, according to an embodiment. To create a healthy control group data structure, the analytics system can receive a plurality of DNA fragments (e.g., cfDNA) from a plurality of healthy individuals. The analytics system can generate 605 a methylation state vector for each fragment, for example via the process 200 of FIG. 2A.

With each fragment's methylation state vector, the analytics system can subdivide 610 the methylation state vector into strings of CpG sites. In some embodiments, the analytics system subdivides 610 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 can result in 4 strings of length 4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system tallies 615 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 610 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site in the reference genome. The analytics system creates 615 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2\hat{ }4 numbers to tally for strings of length 4. Increasing the maximum string length to 5 means that every CpG site has an additional 2\hat{ }4 or 16 numbers to tally, doubling the numbers to tally (and computer memory required) compared to the prior string length. Reducing string size can help keep the data structure creation and performance (e.g., use for later accessing as described below), in terms of computational and storage, reasonable. Second, a statistical consideration to limiting the maximum string length can be to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it uses a significant amount of data that may not be available, and thus can be too sparse for a model to perform appropriately. For example, calculating a probability of anomalousness/cancer conditioned on the prior 100 CpG sites can use counts of strings in the data structure of length 100, ideally some matching exactly the prior 100 methylation states. If only sparse counts of strings of length 100 are available, there can be insufficient data to determine whether a given string of length of 100 in a test sample is anomalous or not.

FIG. 6B is a flowchart describing a process 630 for identifying informatively methylated fragments from an individual, according to an embodiment. In process 630, the analytics system generates 640 methylation state vectors from cfDNA fragments of the subject, e.g., via the process 200 of FIG. 2A. The analytics system can handle each methylation state vector as follows.

For a given methylation state vector, the analytics system enumerates 645 all possibilities of methylation state vectors having the same starting CpG site and same length (i.e., set of CpG sites) in the methylation state vector. As each methylation state is generally either methylated or unmethylated there can be effectively two possible states at each CpG site, and thus the count of distinct possibilities of methylation state vectors can depend on a power of 2, such that a methylation state vector of length n would be associated with 2ⁿ possibilities of methylation state vectors. With methylation state vectors inclusive of indeterminate states for one or more CpG sites, the analytics system may enumerate 630 possibilities of methylation state vectors considering only CpG sites that have observed states.

The analytics system calculates 650 the probability of observing each possibility of methylation state vector for the identified starting CpG site and methylation state vector length by accessing the healthy control group data structure. In some embodiments, calculating the probability of observing a given possibility uses a Markov chain probability to model the joint probability calculation. The Markov model can be trained, at least in part, based upon evaluation of a methylation state of each CpG site in the corresponding plurality of CpG sites of the respective fragment (e.g., nucleic acid methylation fragment) across those nucleic acid methylation fragments in a healthy noncancer cohort dataset that have the corresponding plurality of CpG sites. For example, a Markov model (e.g., a Hidden Markov Model or HMM) is used to determine the probability that a sequence of methylation states (comprising, e.g., “M” or “U”) can be observed for a nucleic acid methylation fragment in a plurality of nucleic acid methylation fragments, given a set of probabilities that determine, for each state in the sequence, the likelihood of observing the next state in the sequence. The set of probabilities can be obtained by training the HMM. Such training can involve computing statistical parameters (e.g., the probability that a first state can transition to a second state (the transition probability) and/or the probability that a given methylation state can be observed for a respective CpG site (the emission probability)), given an initial training dataset of observed methylation state sequences (e.g., methylation patterns). HMMs can be trained using supervised training (e.g., using samples where the underlying sequence as well as the observed states are known) and/or unsupervised training (e.g., Viterbi learning, maximum likelihood estimation, expectation-maximization training, and/or Baum-Welch training). In other embodiments, calculation methods other than Markov chain probabilities are used to determine the probability of observing each possibility of methylation state vector. For example, such calculation method can include a learned representation. The p-value threshold can be between 0.01 and 0.10, or between 0.03 and 0.06. The p-value threshold can be 0.05. The p-value threshold can be less than 0.01, less than 0.001, or less than 0.0001.

The analytics system calculates 655 a p-value score for the methylation state vector using the calculated probabilities for each possibility. In some embodiments, this includes identifying the calculated probability corresponding to the possibility that matches the methylation state vector in question. Specifically, this can be the possibility having the same set of CpG sites, or similarly the same starting CpG site and length as the methylation state vector. The analytics system can sum the calculated probabilities of any possibilities having probabilities less than or equal to the identified probability to generate the p-value score.

This p-value can represent the probability of observing the methylation state vector of the fragment or other methylation state vectors even less probable in the healthy control group. A low p-value score can, thereby, generally correspond to a methylation state vector which is rare in a healthy individual, and which causes the fragment to be labeled an informative fragment, relative to the healthy control group. A high p-value score can generally relate to a methylation state vector is expected to be present, in a relative sense, in a healthy individual. If the healthy control group is a non-cancerous group, for example, a low p-value can indicate that the fragment has an informative methylation pattern relative to the non-cancer group, and therefore possibly indicative of the presence of cancer in the test subject.

As above, the analytics system can calculate p-value scores for each of a plurality of methylation state vectors, each representing a cfDNA fragment in the test sample. To identify which of the fragments have an informative methylation pattern, the analytics system may filter 665 the set of methylation state vectors based on their p-value scores. In some embodiments, filtering is performed by comparing the p-values scores against a threshold and keeping only those fragments below the threshold. This threshold p-value score can be on the order of 0.1, 0.01, 0.001, 0.0001, or similar.

According to example results from the process 600, the analytics system can yield a median (range) of 2,800 (1,500-12,000) fragments with informative methylation patterns for participants without cancer in training, and a median (range) of 3,000 (1,200-420,000) fragments with informative methylation patterns for participants with cancer in training. These filtered sets of fragments with informative methylation patterns may be used for the downstream analyses as described below in Sections IV.B & IV.C.

In some embodiments, the analytics system uses 660 a sliding window to determine possibilities of methylation state vectors and calculate p-values. Rather than enumerating possibilities and calculating p-values for entire methylation state vectors, the analytics system can enumerate possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window can identify the sequential set of CpG sites from the vector within the window starting from the first CpG site in the vector. The analytics system can calculate a p-value score for the window including the first CpG site. The analytics system can then “slide” the window to the second CpG site in the vector, and calculates another p-value score for the second window. Thus, for a window size l and methylation vector length m, each methylation state vector can generate m−l+1 p-value scores. After completing the p-value calculations for each portion of the vector, the lowest p-value score from all sliding windows can be taken as the overall p-value score for the methylation state vector. In other embodiments, the analytics system aggregates the p-value scores for the methylation state vectors to generate an overall p-value score.

Using the sliding window can help to reduce the number of enumerated possibilities of methylation state vectors and their corresponding probability calculations that would otherwise need to be performed. To give a realistic example, it can be for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations can enumerate 2\hat{ }5 (32) possibilities of methylation state vectors, which total results in 50×2\hat{ }5 (1.6×10\hat{ }3) probability calculations. This can result in a vast reduction of calculations to be performed, with no meaningful hit to the accurate identification of informative fragments.

In embodiments with indeterminate states, the analytics system may calculate a p-value score summing out CpG sites with indeterminates states in a fragment's methylation state vector. The analytics system can identify all possibilities that have consensus with the all methylation states of the methylation state vector excluding the indeterminate states. The analytics system may assign the probability to the methylation state vector as a sum of the probabilities of the identified possibilities. As an example, the analytics system can calculate a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states can use calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm operates in linear computational time.

In some embodiments, the computational burden of calculating probabilities and/or p-value scores may be further reduced by caching at least some calculations. For example, the analytics system may cache in transitory or persistent memory calculations of probabilities for possibilities of methylation state vectors (or windows thereof). If other fragments have the same CpG sites, caching the possibility probabilities can allow for efficient calculation of p-score values without needing to re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

One or more nucleic acid methylation fragments can be filtered prior to training region models or cancer classifier. Filtering nucleic acid methylation fragments can comprise removing, from the corresponding plurality of nucleic acid methylation fragments, each respective nucleic acid methylation fragment that fails to satisfy one or more selection criteria (e.g., below or above one selection criteria). The one or more selection criteria can comprise a p-value threshold. The output p-value of the respective nucleic acid methylation fragment can be determined, at least in part, based upon a comparison of the corresponding methylation pattern of the respective nucleic acid methylation fragment to a corresponding distribution of methylation patterns of those nucleic acid methylation fragments in a healthy noncancer cohort dataset that have the corresponding plurality of CpG sites of the respective nucleic acid methylation fragment.

Filtering a plurality of nucleic acid methylation fragments can comprise removing each respective nucleic acid methylation fragment that fails to satisfy a p-value threshold. The filter can be applied to the methylation pattern of each respective nucleic acid methylation fragment using the methylation patterns observed across the first plurality of nucleic acid methylation fragments. Each respective methylation pattern of each respective nucleic acid methylation fragment (e.g., Fragment One, . . . , Fragment N) can comprise a corresponding one or more methylation sites (e.g., CpG sites) identified with a methylation site identifier and a corresponding methylation pattern, represented as a sequence of 1's and 0's, where each “1” represents a methylated CpG site in the one or more CpG sites and each “0” represents an unmethylated CpG site in the one or more CpG sites. The methylation patterns observed across the first plurality of nucleic acid methylation fragments can be used to build a methylation state distribution for the CpG site states collectively represented by the first plurality of nucleic acid methylation fragments (e.g., CpG site A, CpG site B, . . . , CpG site ZZZ). Further details regarding processing of nucleic acid methylation fragments are disclosed in U.S. Provisional patent application Ser. No. 17/191,914, titled “Systems and Methods for Cancer Condition Determination Using Autoencoders,” filed Mar. 4, 2021, which is hereby incorporated herein by reference in its entirety.

The respective nucleic acid methylation fragment may fail to satisfy a selection criterion in the one or more selection criteria when the respective nucleic acid methylation fragment has an informative methylation score that is less than an informative methylation score threshold. In this situation, the informative methylation score can be determined by a mixture model. For example, a mixture model can detect an informative methylation pattern in a nucleic acid methylation fragment by determining the likelihood of a methylation state vector (e.g., a methylation pattern) for the respective nucleic acid methylation fragment based on the number of possible methylation state vectors of the same length and at the same corresponding genomic location. This can be executed by generating a plurality of possible methylation states for vectors of a specified length at each genomic location in a reference genome. Using the plurality of possible methylation states, the number of total possible methylation states and subsequently the probability of each predicted methylation state at the genomic location can be determined. The likelihood of a sample nucleic acid methylation fragment corresponding to a genomic location within the reference genome can then be determined by matching the sample nucleic acid methylation fragment to a predicted (e.g., possible) methylation state and retrieving the calculated probability of the predicted methylation state. An informative methylation score can then be calculated based on the probability of the sample nucleic acid methylation fragment.

The respective nucleic acid methylation fragment can fail to satisfy a selection criterion in the one or more selection criteria when the respective nucleic acid methylation fragment has less than a threshold number of residues. The threshold number of residues can be between 10 and 50, between 50 and 100, between 100 and 150, or more than 150. The threshold number of residues can be a fixed value between 20 and 90. The respective nucleic acid methylation fragment may fail to satisfy a selection criterion in the one or more selection criteria when the respective nucleic acid methylation fragment has less than a threshold number of CpG sites. The threshold number of CpG sites can be 4, 5, 6, 7, 8, 9, or 10. The respective nucleic acid methylation fragment can fail to satisfy a selection criterion in the one or more selection criteria when a genomic start position and a genomic end position of the respective nucleic acid methylation fragment indicates that the respective nucleic acid methylation fragment represents less than a threshold number of nucleotides in a human genome reference sequence.

The filtering can remove a nucleic acid methylation fragment in the corresponding plurality of nucleic acid methylation fragments that has the same corresponding methylation pattern and the same corresponding genomic start position and genomic end position as another nucleic acid methylation fragment in the corresponding plurality of nucleic acid methylation fragments. This filtering step can remove redundant fragments that are exact duplicates, including, in some instances, PCR duplicates. The filtering can remove a nucleic acid methylation fragment that has the same corresponding genomic start position and genomic end position and less than a threshold number of different methylation states as another nucleic acid methylation fragment in the corresponding plurality of nucleic acid methylation fragments. The threshold number of different methylation states used for retention of a nucleic acid methylation fragment can be 1, 2, 3, 4, 5, or more than 5. For example, a first nucleic acid methylation fragment having the same corresponding genomic start and end position as a second nucleic acid methylation fragment but having at least 1, at least 2, at least 3, at least 4, or at least 5 different methylation states at a respective CpG site (e.g., aligned to a reference genome) is retained. As another example, a first nucleic acid methylation fragment having the same methylation state vector (e.g., methylation pattern) but different corresponding genomic start and end positions as a second nucleic acid methylation fragment is also retained.

The filtering can remove assay artifacts in the plurality of nucleic acid methylation fragments. The removal of assay artifacts can comprise removing sequence reads obtained from sequenced hybridization probes and/or sequence reads obtained from sequences that failed to undergo conversion during bisulfite conversion. The filtering can remove contaminants (e.g., due to sequencing, nucleic acid isolation, and/or sample preparation).

The filtering can remove a subset of methylation fragments from the plurality of methylation fragments based on mutual information filtering of the respective methylation fragments against the cancer state across the plurality of training subjects. For example, mutual information can provide a measure of the mutual dependence between two conditions of interest sampled simultaneously. Mutual information can be determined by selecting an independent set of CpG sites (e.g., within all or a portion of a nucleic acid methylation fragment) from one or more datasets and comparing the probability of the methylation states for the set of CpG sites between two sample groups (e.g., subsets and/or groups of genotypic datasets, biological samples, and/or subjects). A mutual information score can denote the probability of the methylation pattern for a first condition versus a second condition at the respective region in the respective frame of the sliding window, thus indicating the discriminative power of the respective region. A mutual information score can be similarly calculated for each region in each frame of the sliding window as it progresses across the selected sets of CpG sites and/or the selected genomic regions. Further details regarding mutual information filtering are disclosed in U.S. patent application Ser. No. 17/119,606, titled “Cancer Classification using Patch Convolutional Neural Networks,” filed Dec. 11, 2020, which is hereby incorporated herein by reference in its entirety.

IV.A.ii. Hypermethylated Fragments and Hypomethylated Fragments

In some embodiments, the analytics system identifies 670 determines hypomethylated fragments or hypermethylated fragments from the filtered set as informative fragments. The analytics system identifies hypermethylated fragments having over a threshold number of CpG sites and over a threshold percentage of the CpG sites methylated. The analytics system identifies hypomethylated fragments having over the threshold number of CpG sites and over a threshold percentage of CpG sites unmethylated. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

### IV.B. Training of Cancer Classifier

FIG. 7A is a flowchart describing a process 700 of training a cancer classifier, according to an embodiment. The analytics system obtains 710 a plurality of training samples each having a set of informative fragments and a label of a cancer type. The plurality of training samples can include any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

The analytics system determines 720, for each training sample, a feature vector based on the set of informative fragments of the training sample. The analytics system can calculate an informative score for each CpG site in an initial set of CpG sites. The initial set of CpG sites may be all CpG sites in the human genome or some portion thereof—which may be on the order of 10⁴, 10⁵, 10⁶, 10⁷, 10⁸, etc. In one embodiment, the analytics system defines the informative score for the feature vector with a binary scoring based on whether there is an informative fragment in the set of informative fragments that encompasses the CpG site. In another embodiment, the analytics system defines the informative score based on a count of informative fragments overlapping the CpG site. In one example, the analytics system may use a trinary scoring assigning a first score for lack of presence of informative fragments, a second score for presence of a few informative fragments, and a third score for presence of more than a few informative fragments. For example, the analytics system counts 5 informative fragment in a sample that overlap the CpG site and calculates an informative score based on the count of 5.

Once all informative scores are determined for a training sample, the analytics system can determine the feature vector as a vector of elements including, for each element, one of the informative scores associated with one of the CpG sites in an initial set. The analytics system can normalize the informative scores of the feature vector based on a coverage of the sample. Here, coverage can refer to a median or average sequencing depth over all CpG sites covered by the initial set of CpG sites used in the classifier, or based on the set of informative fragments for a given training sample.

As an example, reference is now made to FIG. 7B illustrating a matrix of training feature vectors 722. In this example, the analytics system has identified CpG sites [K] 726 for consideration in generating feature vectors for the cancer classifier. The analytics system selects training samples [N] 724. The analytics system determines a first informative score 728 for a first arbitrary CpG site [k1] to be used in the feature vector for a training sample [n1]. The analytics system checks each informative fragment in the set of informative fragments. If the analytics system identifies at least one informative fragment that includes the first CpG site, then the analytics system determines the first informative score 728 for the first CpG site as 1, as illustrated in FIG. 7B. Considering a second arbitrary CpG site [k2], the analytics system similarly checks the set of informative fragments for at least one that includes the second CpG site [k2]. If the analytics system does not find any such informative fragment that includes the second CpG site, the analytics system determines a second informative score 729 for the second CpG site [k2] to be 0, as illustrated in FIG. 7B. Once the analytics system determines all the informative scores for the initial set of CpG sites, the analytics system determines the feature vector for the first training sample [n1] including the informative scores with the feature vector including the first informative score 728 of 1 for the first CpG site [k1] and the second informative score 729 of 0 for the second CpG site [k2] and subsequent informative scores, thus forming a feature vector [1, 0, . . . ].

Additional approaches to featurization of a sample can be found in: U.S. application Ser. No. 15/931,022 entitled “Model-Based Featurization and Classification;” U.S. application Ser. No. 16/579,805 entitled “Mixture Model for Targeted Sequencing;” U.S. application Ser. No. 16/352,602 entitled “Anomalous Fragment Detection and Classification;” and U.S. application Ser. No. 16/723,716 entitled “Source of Origin Deconvolution Based on Methylation Fragments in Cell-Free DNA Samples;” all of which are incorporated by reference in their entirety.

The analytics system may further limit the CpG sites considered for use in the cancer classifier. The analytics system computes 730, for each CpG site in the initial set of CpG sites, an information gain based on the feature vectors of the training samples. From step 720, each training sample has a feature vector that may contain an informative score all CpG sites in the initial set of CpG sites which could include up to all CpG sites in the human genome. However, some CpG sites in the initial set of CpG sites may not be as informative as others in distinguishing between cancer types, or may be duplicative with other CpG sites.

In one embodiment, the analytics system computes 730 an information gain for each cancer type and for each CpG site in the initial set to determine whether to include that CpG site in the classifier. The information gain is computed for training samples with a given cancer type compared to all other samples. For example, two random variables ‘informative fragment’ (‘IF’) and ‘cancer type’ (‘CT’) are used. In one embodiment, IF is a binary variable indicating whether there is an informative fragment overlapping a given CpG site in a given samples as determined for the informative score/feature vector above. CT is a random variable indicating whether the cancer is of a particular type. The analytics system computes the mutual information with respect to CT given AF. That is, how many bits of information about the cancer type are gained if it is known whether there is an informative fragment overlapping a particular CpG site. In practice, for a first cancer type, the analytics system computes pairwise mutual information gain against each other cancer type and sums the mutual information gain across all the other cancer types.

For a given cancer type, the analytics system can use this information to rank CpG sites based on how cancer specific they are. This procedure can be repeated for all cancer types under consideration. If a particular region is commonly informatively methylated in training samples of a given cancer but not in training samples of other cancer types or in healthy training samples, then CpG sites overlapped by those informative fragments can have high information gains for the given cancer type. The ranked CpG sites for each cancer type can be greedily added (selected) 740 to a selected set of CpG sites based on their rank for use in the cancer classifier.

In additional embodiments, the analytics system may consider other selection criteria for selecting informative CpG sites to be used in the cancer classifier. One selection criterion may be that the selected CpG sites are above a threshold separation from other selected CpG sites. For example, the selected CpG sites are to be over a threshold number of base pairs away from any other selected CpG site (e.g., 100 base pairs), such that CpG sites that are within the threshold separation are not both selected for consideration in the cancer classifier.

In one embodiment, according to the selected set of CpG sites from the initial set, the analytics system may modify 750 the feature vectors of the training samples as needed. For example, the analytics system may truncate feature vectors to remove informative scores corresponding to CpG sites not in the selected set of CpG sites.

With the feature vectors of the training samples, the analytics system may train the cancer classifier in any of a number of ways. The feature vectors may correspond to the initial set of CpG sites from step 720 or to the selected set of CpG sites from step 750. In one embodiment, the analytics system trains 760 a binary cancer classifier to distinguish between cancer and non-cancer based on the feature vectors of the training samples. In this manner, the analytics system uses training samples that include both non-cancer samples from healthy individuals and cancer samples from subjects. Each training sample can have one of the two labels “cancer” or “non-cancer.” In this embodiment, the classifier outputs a cancer prediction indicating the likelihood of the presence or absence of cancer.

In another embodiment, the analytics system trains 770 a multiclass cancer classifier to distinguish between many cancer types (also referred to as tissue of origin (TOO) labels). Cancer types can include one or more cancers and may include a non-cancer type (may also include any additional other diseases or genetic disorders, etc.). To do so, the analytics system can use the cancer type cohorts and may also include or not include a non-cancer type cohort. In this multi-cancer embodiment, the cancer classifier is trained to determine a cancer prediction (or, more specifically, a TOO prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample, also may be referred to as a TOO prediction indicating one or more TOO labels, e.g., a first TOO label with the highest prediction value, a second TOO label with the second highest prediction value, etc. Continuing with the example above and given the percentages, in this example the system may determine that the sample has breast cancer given that breast cancer has the highest likelihood.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier can be sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

The classifier can include a logistic regression algorithm, a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

### IV.C. Deployment of Cancer Classifier

During use of the cancer classifier, the analytics system can obtain a test sample from a subject of unknown cancer type. The analytics system may process the test sample comprised of DNA molecules with any combination of the processes 200 and 630 to achieve a set of informative fragments. The analytics system can determine a test feature vector for use by the cancer classifier according to similar principles discussed in the process 700. The analytics system can calculate an informative score for each CpG site in a plurality of CpG sites in use by the cancer classifier. For example, the cancer classifier receives as input feature vectors inclusive of informative scores for 1,000 selected CpG sites. The analytics system can thus determine a test feature vector inclusive of informative scores for the 1,000 selected CpG sites based on the set of informative fragments. The analytics system can calculate the informative scores in a same manner as the training samples. In some embodiments, the analytics system defines the informative score as a binary score based on whether there is a hypermethylated or hypomethylated fragment in the set of informative fragments that encompasses the CpG site.

The analytics system can then input the test feature vector into the cancer classifier. The function of the cancer classifier can then generate a cancer prediction based on the classification parameters trained in the process 700 and the test feature vector. In the first manner, the cancer prediction can be binary and selected from a group consisting of “cancer” or non-cancer;” in the second manner, the cancer prediction is selected from a group of many cancer types and “non-cancer.” In additional embodiments, the cancer prediction has predictions values for each of the many cancer types. Moreover, the analytics system may determine that the test sample is most likely to be of one of the cancer types. Following the example above with the cancer prediction for a test sample as 65% likelihood of breast cancer, 25% likelihood of lung cancer, and 10% likelihood of non-cancer, the analytics system may determine that the test sample is most likely to have breast cancer. In another example, where the cancer prediction is binary as 60% likelihood of non-cancer and 40% likelihood of cancer, the analytics system determines that the test sample is most likely not to have cancer. In additional embodiments, the cancer prediction with the highest likelihood may still be compared against a threshold (e.g., 40%, 50%, 60%, 70%) in order to call the test subject as having that cancer type. If the cancer prediction with the highest likelihood does not surpass that threshold, the analytics system may return an inconclusive result.

In additional embodiments, the analytics system chains a cancer classifier trained in step 760 of the process 700 with another cancer classifier trained in step 770 or the process 700. The analytics system can input the test feature vector into the cancer classifier trained as a binary classifier in step 760 of the process 700. The analytics system can receive an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types. The multiclass cancer classifier can receive the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%.

According to generalized embodiment of binary cancer classification, the analytics system can determine a cancer score for a test sample based on the test sample's sequencing data (e.g., methylation sequencing data, SNP sequencing data, other DNA sequencing data, RNA sequencing data, etc.). The analytics system can compare the cancer score for the test sample against a binary threshold cutoff for predicting whether the test sample likely has cancer. The binary threshold cutoff can be tuned using TOO thresholding based on one or more TOO subtype classes. The analytics system may further generate a feature vector for the test sample for use in the multiclass cancer classifier to determine a cancer prediction indicating one or more likely cancer types.

The classifier may be used to determine the disease state of a test subject, e.g., a subject whose disease status is unknown. The method can include obtaining a test genomic data construct (e.g., single time point test data), in electronic form, that includes a value for each genomic characteristic in the plurality of genomic characteristics of a corresponding plurality of nucleic acid fragments in a biological sample obtained from a test subject. The method can then include applying the test genomic data construct to the test classifier to thereby determine the state of the disease condition in the test subject. The test subject may not be previously diagnosed with the disease condition.

The classifier can be a temporal classifier that uses at least (i) a first test genomic data construct generated from a first biological sample acquired from a test subject at a first point in time, and (ii) a second test genomic data construct generated from a second biological sample acquired from a test subject at a second point in time.

The trained classifier can be used to determine the disease state of a test subject, e.g., a subject whose disease status is unknown. In this case, the method can include obtaining a test time-series data set, in electronic form, for a test subject, where the test time-series data set includes, for each respective time point in a plurality of time points, a corresponding test genotypic data construct including values for the plurality of genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points. The method can then include applying the test genotypic data construct to the test classifier to thereby determine the state of the disease condition in the test subject. The test subject may not be previously diagnosed with the disease condition.

### V. Applications

In some embodiments, the methods, analytics systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has cancer. In other embodiments, the likelihood or probability score can be assessed at multiple different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the probability score exceeds a threshold, a physician can prescribe an appropriate treatment. In additional embodiments, the methods, analytics system, and/or classifiers can be implemented to detect contamination sources in the sample processing and analysis workflow. Upon detection of contamination and/or contamination sources, the analytics system can aid in performing remedial measures to mitigate the contamination and the negative effects thereof (e.g., skewing results, biasing training of the classifiers, etc.).

### V.A. WBC Contamination

In some embodiments, the methods and/or classifier of the present invention are used to detect WBC contamination, e.g., as described by the various methodologies in Section III.

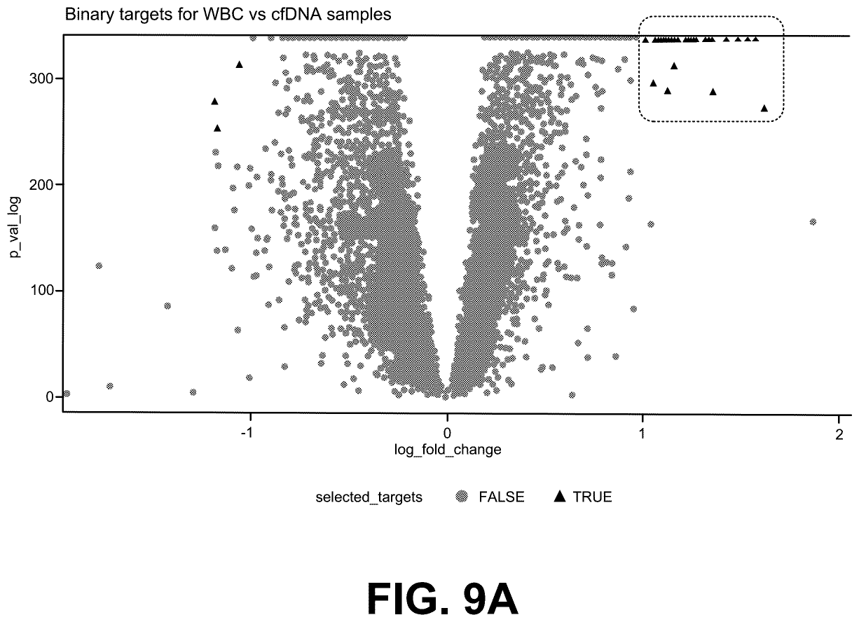

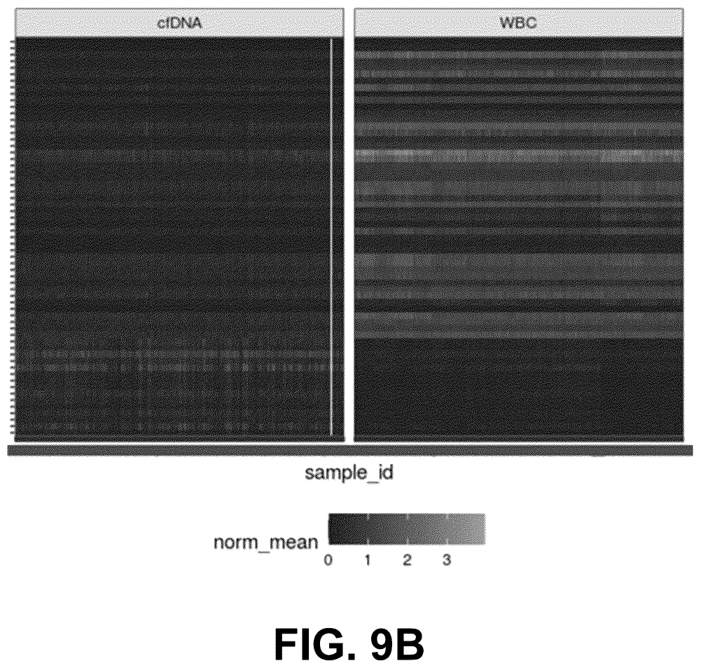

According to one or more example implementations of the first methodology of WBC contamination detection, FIG. 9A through FIG. 14 describe example results.

FIG. 9A illustrates a volcano plot of ratios of coverage for an initial set of genomic loci considered. The ratios of coverage are calculated by dividing coverages between WBC samples and cfDNA samples. The x-axis plots the log-fold-change of ratios of coverage. For example, a log-fold-change with a log scale of base 2 results in a value of 1.5 equating to a 2\hat{ }1.5 equal to approximately a 2.82 multiplicative factor. The y-axis plots significance of the genomic loci, i.e., as logarithmic of p-value. Higher up on the y-axis indicates higher statistical significance. Here, genomic loci were selected within the upper right square (e.g., denoted as triangles) as those having ratios of coverage above a threshold and high statistical significance.

FIG. 9B illustrates a visual comparison of differential coverage at the selected genomic loci between cfDNA samples and WBC samples. The genomic loci are listed vertically, e.g., along the y-axis, with samples strewn across horizontally, e.g., along the x-axis. The legend below indicates darker referring to low normalized coverage with lighter referring to high normalized coverage. Noticeably, there are consistent horizontal light bands on the WBC sample side that do not have corresponding light bands on the cfDNA sample side. This demonstrates the discriminatory power of the feature set of genomic loci.

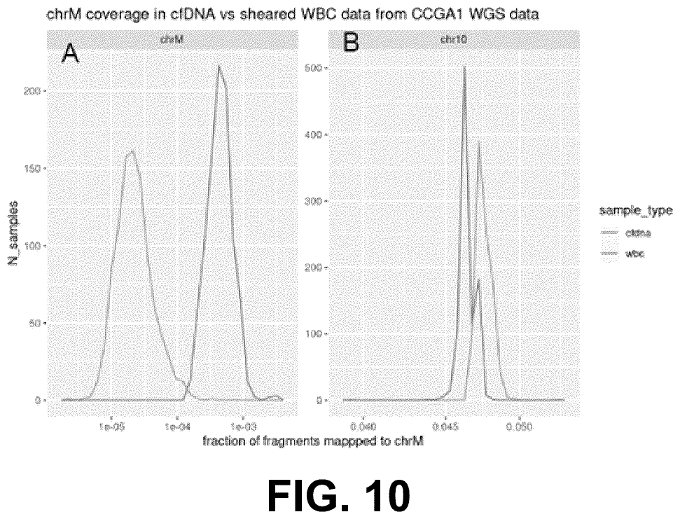

FIG. 10 illustrates the one genomic locus in mitochondrial DNA with stark difference in representation between cfDNA samples and WBC samples. Distribution of fragments mapping to chrM (mitochondrial DNA) and chr10 (control) in whole genome sequencing data for cfDNA and sheared white blood cell gDNA from CCGA1 samples. The left graph shows a proportion of cfDNA mapping to chrM (red and to the left) is significantly less (˜17×) than what is observed in WBC gDNA (blue and to the right) (note the log 10 scale on the x-axis). The right graph, as a control, shows the difference between the proportion of fragments mapping to chr10 between cfDNA and WBC DNA is much more similar (within 5%). The low proportion of cfDNA fragments mapping to chrM is likely due to the lack of nucleosomes in the mitochondria to protect this DNA from degradation in vivo after shedding from the cell, unlike the majority of cfDNA which is derived from nuclear DNA which is typically wrapped around nucleosomes. Such difference in coverage in chrM can be exploited for monitoring WBC contamination in clinical samples since gDNA lysed from WBC into plasma that has been collected in EDTA or STRECK tubes is not degraded as rapidly as in peripheral blood. As a result, an increased proportion of fragments mapping to chrM e.g., above 1e⁻⁴ may represent WBC contamination in the sample.

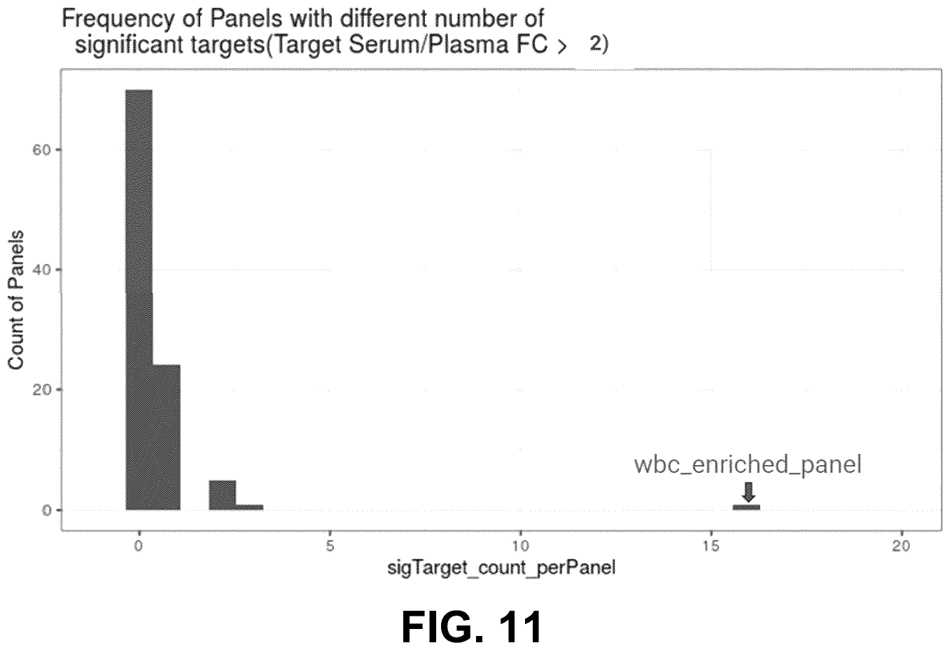

FIG. 11 illustrates a comparison of significant genomic loci in a feature set of genomic loci compared to randomly selected candidate sets. FIG. 11 illustrates a bar chart quantifying how many candidate sets (out of 100 candidate sets) had various numbers of significant genomic loci. The threshold to be considered a significant genomic loci is whether the ratio of coverage of the genomic locus by WBC samples to the coverage of the genomic locus by cfDNA samples was greater than 2. Nearly 70 of the candidate sets had zero significant genomic loci, and subsequently had little to no predictive ability in assessing presence of WBC contamination. There were around 22 candidate sets that had one significant genomic locus, some candidate sets with more than one significant genomic locus but less than five. The feature set (e.g., as identified by the feature identification 300 in FIG. 3A) yielded at least 15 significant genomic loci. This bar chart illustrates the improvement of the feature set compared to randomly selected markers or genomic loci.

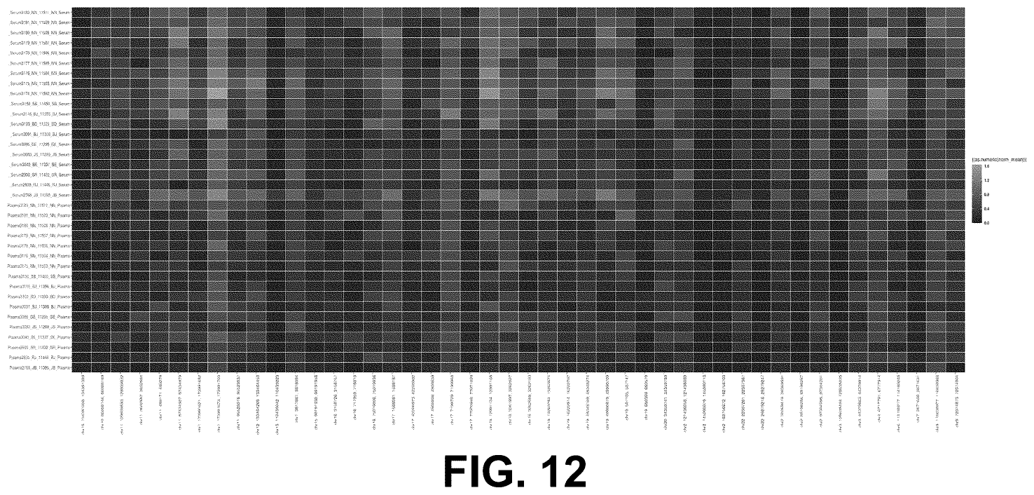

FIG. 12 illustrates example paired WBC samples and cfDNA samples from the same subjects assessed over the feature set of genomic loci (e.g., as identified by the feature identification 300 in FIG. 3A). Of note, there is not one target or genomic locus that can solely predict WBC contamination. Rather, across the various genomic loci, some paired samples have similar coverage in the WBC sample and cfDNA sample, which would not provide any discriminatory power. Nonetheless, the aggregation of the various genomic loci in the feature set work in conjunction to yield the predictive power of the coverage-based WBC contamination methodology.

In a first set of validation experiments, samples enriched with longer fragments (longest top 1% of sequence reads) were tested against the feature set of genomic loci for coverage-based WBC contamination against randomly selected candidate sets of genomic loci. The feature set (e.g., as identified in the feature identification 300 of FIG. 3A) identified 3 outlier samples out of 100 samples. The results of the candidate sets are summed in the following Table 2:

TABLE 2

| Number of Outlier Samples | Number of Candidate Sets |
|---|---|
| (out of 100 samples) | (500 total) |
| 0 | 262 |
| 1 | 220 |
| 2 | 17 |
| 3 | 1 |

The majority of the candidate sets predicted no outlier samples, i.e., no samples to be WBC contaminated, with just under half of the candidate sets predicting one outlier sample. About 3.5% of the candidate sets predicted two outlier samples, and only one candidate set out of the 500 (0.2%) predicted three outlier samples.

In a second set of validation experiments, samples enriched with longer fragments (longest top 1% of sequence reads) were tested against the feature set of genomic loci for coverage-based WBC contamination against randomly selected candidate sets of genomic loci. The feature set (e.g., as identified in the feature identification 300 of FIG. 3A) identified 7 outlier samples out of 62 samples. The results of the candidate sets are summed in the following Table 3:

TABLE 3

| Number of Outlier Samples | Number of Candidate Sets |
|---|---|
| (out of 100 samples) | (100 total) |
| 0 | 60 |
| 1 | 36 |
| 2 | 4 |

The majority of the candidate sets (60%) predicted no outlier samples, i.e., no samples to be WBC contaminated, approximately a third of the candidate sets (36%) predicting one outlier sample, and 4% of the candidate sets predicted two outlier samples. No candidate sets predicted beyond two outlier samples, and yet the feature set identified 7 outlier samples.

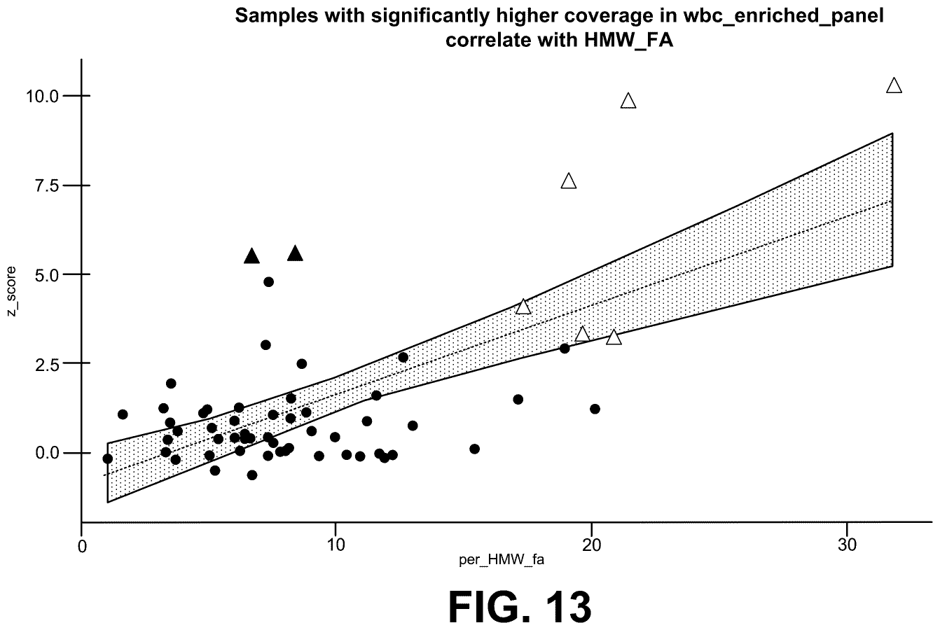

FIG. 13 illustrates samples identified as WBC contaminated by the coverage-based WBC contamination detection. Along the x-axis, samples are plotted based on high molecular weight. Along the y-axis, samples are plotted based on z-score as determined by the inference 370 in FIG. 3C. The white triangles refer to samples that were most confidently determined to be outliers, i.e., WBC contaminated, with the feature set of genomic loci (e.g., as identified according to feature identification 300 in FIG. 3A). The black triangles refer to samples that were, though identified as outliers by the feature set of genomic loci, were most confidently determined to be outliers by random candidate sets. Many of the white triangles were not identified as WBC contaminated by other random candidate sets.

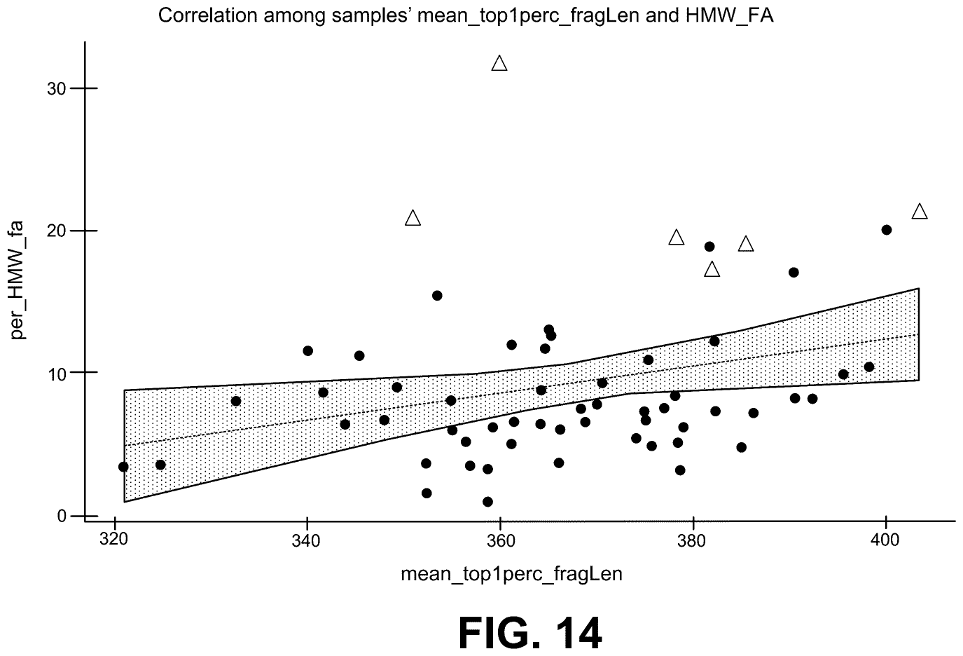

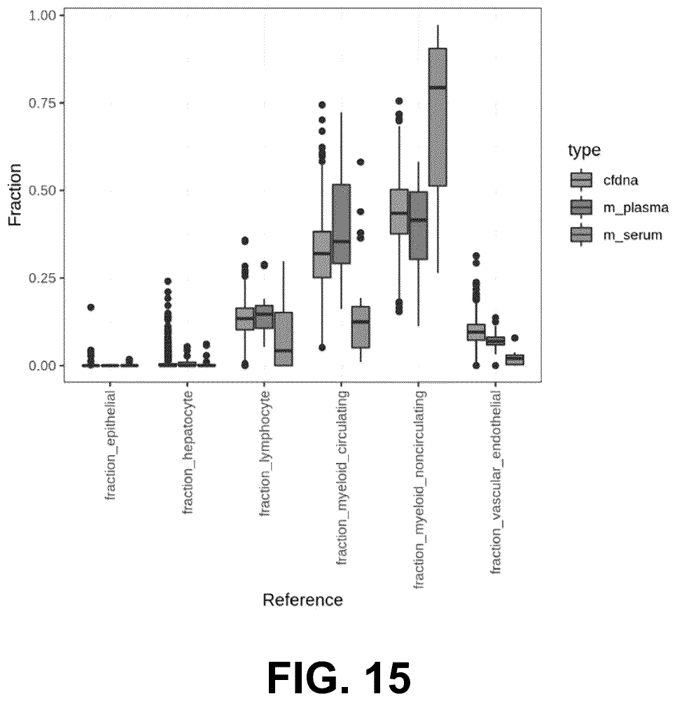

FIG. 14 illustrates samples with longest fragments (top 1%) identified as WBC contaminated by the coverage-based WBC contamination detection. Along the x-axis, samples are plotted based on overall fragment length in the sample. Along the y-axis, samples are plotted based on z-score as determined by the inference 370 in FIG. 3C. The white triangles refer to samples that were most confidently determined to be outliers, i.e., WBC contaminated, with the feature set of genomic loci (e.g., as identified according to feature identification 300 in FIG. 3A). Importantly, fragment length is not a reliable indicator to WBC contamination. Many samples above 360 in mean top 1% of fragment lengths were not WBC contaminated. And, moreover, some samples below the 360 mark were WBC contaminated. Utilizing a fragment length cutoff would not be accurate in WBC contamination detection.

According to one or more example implementations of the second methodology of WBC contamination detection, FIG. 15 through FIG. 19 describe example results.

FIG. 15 illustrates fractional contributions of various tissue types for three different sample types: cfDNA samples, purified plasma samples, and serum samples containing only WBC DNA. The tissue types include epithelial cell type, hepatocyte cell type, lymphocyte cell type, myeloid circulating tissue type, myeloid non-circulating tissue type, and vascular endothelial cell type. Of note, plasma fractional contributions are dissimilar from serum fractional contributions. This is a proof of concept to the implementation of the second methodology.

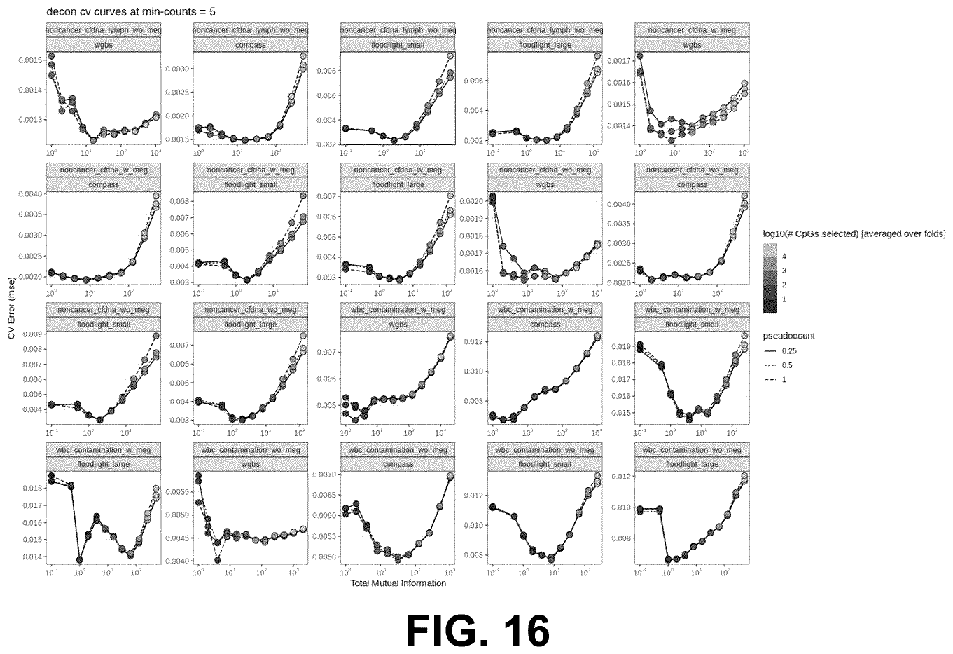

FIG. 16 illustrates information gain across a set of genomic loci evaluated for inclusion as part of the deconvolution model (e.g., as trained in FIG. 4A). The x-axis plots mutual information gained during training of the deconvolution model, and the y-axis plots predictive error (mean squared error). Each graph represents the mutual information gained for a single genomic locus or deconvolution marker.

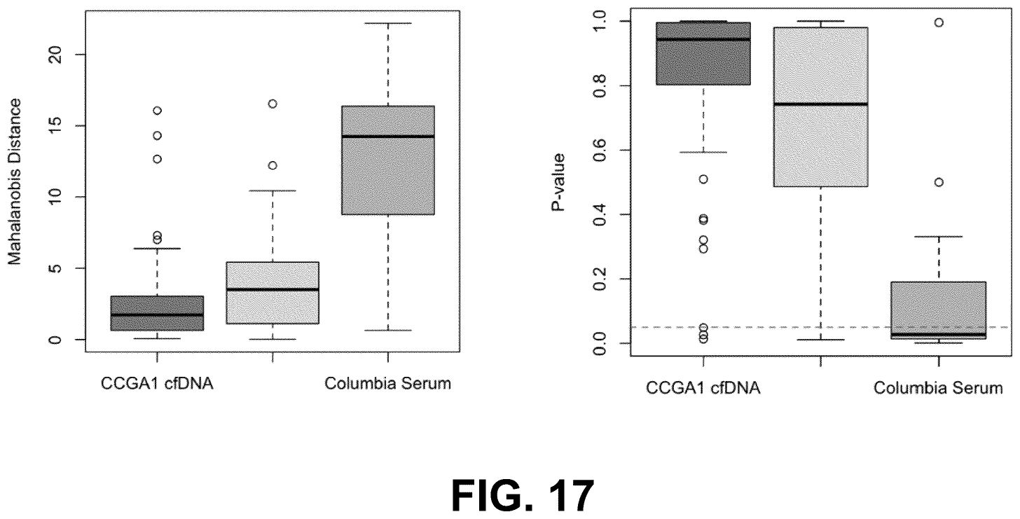

FIG. 17 illustrates box plots showing the predictive power of the methylation-based WBC contamination detection methodology. The left graph's y-axis plots Mahalanobis distance, i.e., distance from the mean of tissue type fractions for a null distribution of cfDNA samples from a non-cancer population. The right graph's y-axis plots p-value as a contamination metric for calling WBC contamination in samples. The contamination threshold is set at 0.05. The green box plots (on the left of each graph), labeled CCGA1 cfDNA, refers to cfDNA samples from the CCGA1 dataset. The Mahalanobis distance (used in calculating the contamination metric) is relatively low of the cfDNA samples, and correspondingly the contamination metric is generally very high, with only a select few outliers below the contamination threshold. These samples could have been WBC contaminated without any affirmative detection. The orange box plots (on the right of each graph), labeled Columbia Serum, refers to WBC gDNA samples. In contrast to the cfDNA samples, for the WBC samples, the Mahalanobis distance is large and the contamination metric as the p-value is generally lower. Of note, the majority of the WBC gDNA samples would have been called with WBC contamination with p-values below the contamination threshold of 0.05.

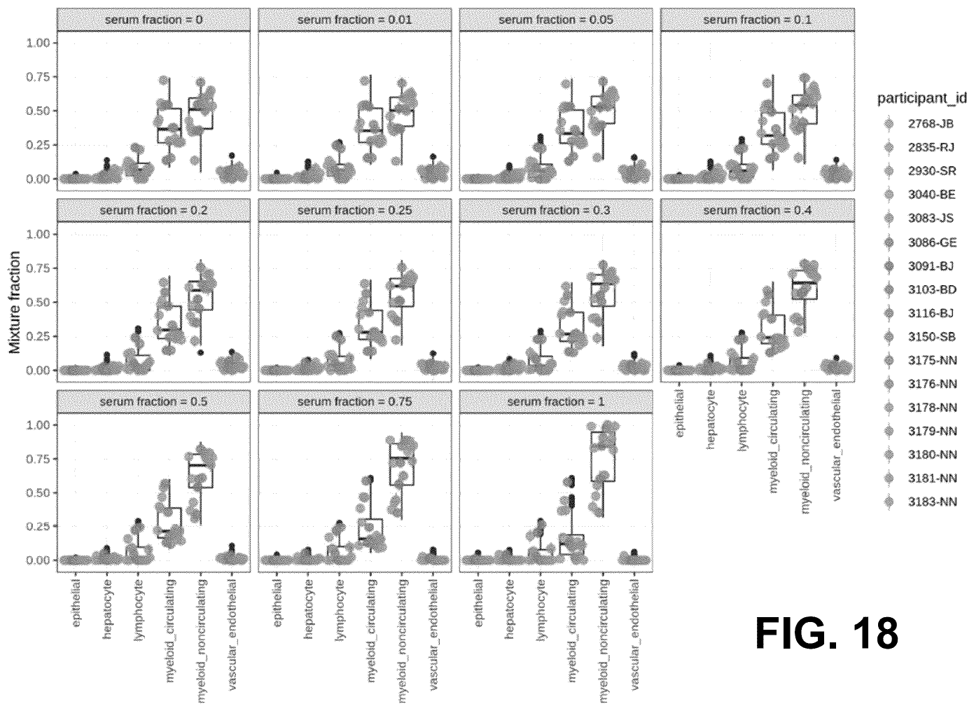

FIG. 18 illustrates titrated samples mixed with cFDNA and WBC serum, according to example implementations. Each graph has the same axes, x-axis indicating tissue types (epithelial cell type, hepatocyte cell type, lymphocyte cell type, myeloid circulating tissue type, myeloid non-circulating tissue type, and vascular endothelial cell type), with the y-axis indicating the fractional contribution of the various tissue types. The baseline experiment (top left graph with no added WBC serum) indicates, on average, 50% fractional contribution from myeloid non-circulating tissue type, ˜37.5% fractional contribution from myeloid circulative tissue type, ˜5% fractional contribution of lymphocyte cell type, and <5% fractional contribution for the remaining tissue types. As more Serum is titrated into the samples, there was an increase fractional contribution of myeloid non-circulating tissue type with decreases in fractional contribution of myeloid non-circulating tissue type and fractional contribution of lymphocyte cell type. These experiments validate expectations in FIG. 15.

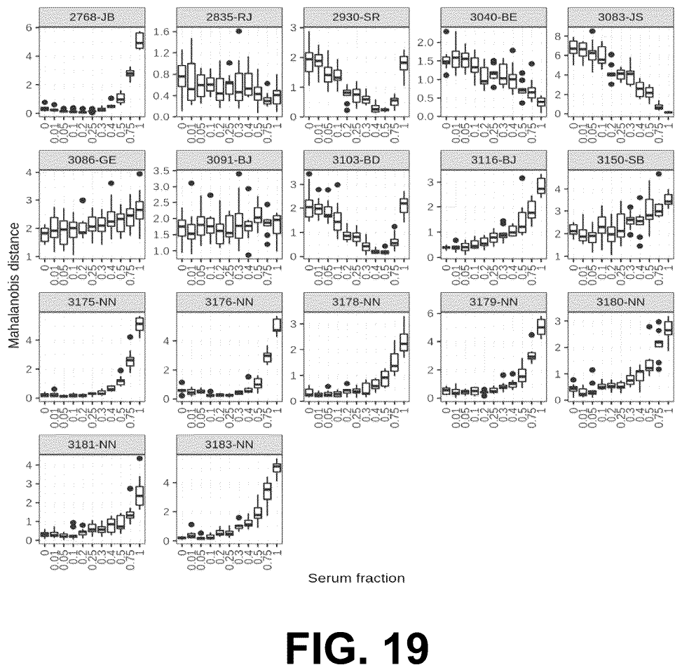

FIG. 19 illustrates titrated samples with calculated Mahalanobis distance, according to the second methodology. Each graph corresponds to a single subject with example titrations of WBC Serum into cfDNA Plasma. The x-axis denotes the titration level of WBC Serum, with the y-axis denoting Mahalanobis distance. The majority of samples demonstrate increasing Mahalanobis distance, indicating further departure from the average tissue type fractions in the null distribution of cfDNA samples from non-cancer subjects. This corroborates the predictive power of utilizing Mahalanobis distance in the contamination metric calculation.

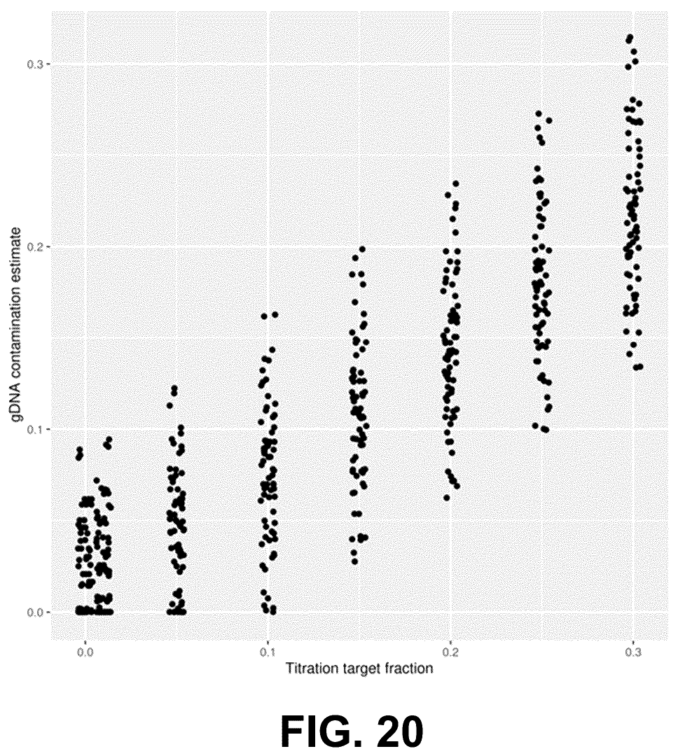

According to one or more example implementations of the third methodology of WBC contamination detection, FIG. 20 describes example results.

FIG. 20 illustrates titrated samples quantified for WBC contamination, according to the third methodology. Each dot represents a titrated sample comprising a set fraction of WBC serum DNA titrated into cfDNA plasma. The x-axis plots the fraction of WBC serum in each sample, whereas the y-axis plots the predicted gDNA contamination based on the model trained according to the third methodology described in FIGS. 5A & 5B. Of note, the model accurately predicted increased WBC gDNA contamination gradually with each titration class. For example, the spread of WBC gDNA contamination in the titration class of 0.10 of WBC gDNA was from 0.00 to ˜0.16, whereas the spread of WBC gDNA contamination in the titration class of 0.30 of WBC gDNA was from ˜0.13 to ˜3.2. The spreads are also fairly consistent across titration classes.

### V.B. Early Detection of Cancer

In some embodiments, the methods and/or classifier of the present invention are used to detect the presence or absence of cancer in a subject suspected of having cancer. For example, a classifier (e.g., as described above in Section IV and exampled in Section V) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e., binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50%5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

### V.C. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein). The classification may further quantify tumor burden to assess change over time.

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction, then the treatment is considered to have been successful. However, if the second cancer prediction increases compared to the first cancer prediction, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 50 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 5 months, at least once every 6 months, at least once a year, at least once every 2 years, at least once every 3 years, at least once every 4 years, or at least once every 5 years.

### V.D. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician can prescribe an appropriate treatment (e.g., a resection surgery, radiation therapy, chemotherapy, and/or immunotherapy).

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### V.E. Kit Implementation

Also disclosed herein are kits for performing the methods described above including the methods relating to the cancer classifier. The kits may include one or more collection vessels for collecting a sample from the individual comprising genetic material. The sample can include blood, plasma, serum, urine, fecal, saliva, other types of bodily fluids, or any combination thereof. Such kits can include reagents for isolating nucleic acids from the sample. The reagents can further include reagents for sequencing the nucleic acids including buffers and detection agents. In one or more embodiments, the kits may include one or more sequencing panels comprising probes for targeting particular genomic regions (e.g., one or more of the regions found in Table 1), particular mutations, particular genetic variants, or some combination thereof. In other embodiments, samples collected via the kit are provided to a sequencing laboratory that may use the sequencing panels to sequence the nucleic acids in the sample. The WBC contamination detection may be applied to various configurations of the kit, to minimize WBC contamination potentially originating from components of the kit. For example, experiments may be run comparing types of collection vessels. WBC contamination can be assessed and compared between the types of collection vessels to identify an optimal type that minimizes WBC contamination.

A kit can further include instructions for use of the reagents included in the kit. For example, a kit can include instructions for collecting the sample, extracting the nucleic acid from the test sample. Example instructions can be the order in which reagents are to be added, centrifugal speeds to be used to isolate nucleic acids from the test sample, how to amplify nucleic acids, how to sequence nucleic acids, or any combination thereof. The instructions may further illumine how to operate a computing device as the analytics system 200, for the purposes of performing the steps of any of the methods described.

In addition to the above components, the kit may include computer-readable storage media storing computer software for performing the various methods described throughout the disclosure. One form in which these instructions can be present is as printed information on a suitable medium or substrate, e.g., a piece or pieces of paper on which the information is printed, in the packaging of the kit, in a package insert. Yet another means would be a computer readable medium, e.g., diskette, CD, hard-drive, network data storage, on which the instructions have been stored in the form of computer code. Yet another means that can be present is a website address or QR code which can be used via the internet to access the information at a removed site.

### V.F. Contamination Source Detection & Mitigation

In some embodiments, the methods and/or classifier of the present invention are used to detect WBC contamination, e.g., as described by the various methodologies in Section III.

The analytics system may leverage the WBC contamination workflow to identify a source of the WBC contamination. To identify the source, the analytics system may isolate one or more variables of the sample processing workflow. The analytics system may process a first of samples with a first sample processing workflow and a second set of samples with a second sample processing workflow, with the second sample processing workflow different in the one or more variables being assessed. For example, the second sample processing workflow may include a different protocol, a different clinical product, or a different sequencing device. Protocols may include steps undertaken in processing the sample, e.g., centrifugation, storage temperature, storage duration, etc. Clinical products are manufactured products used in the sample processing workflow and may include, e.g., any vessel, any chemical, any compound, any buffer, any solution, any enzyme, or any other product used in the workflow. The sequencing device may generally include the sequencer, but may also include other devices related to the sequencing process. The sample processing workflow may further include other laboratory devices, e.g., centrifuge, storage devices, other laboratory devices for sample processing, etc.

The analytics system applies a contamination model (e.g., any of the contamination models described) to the samples from both sequencing workflows. The analytics system may determine an aggregate metric for each sequencing workflow based on the contamination metrics of the respective sample sets. For example, the aggregate metric may be an average, a weighted average, a count or a percentage of samples above a contamination threshold, or some combination thereof. The analytics system may compare the aggregate contamination metrics to concretely identify the second sample processing workflow as contributing to the WBC contamination. If there is a significant difference, then the analytics system may identify the source based on what variables were different between the first and the second sample processing workflows. Remedial measures may also be implemented.

With the contamination source identified, the analytics system may determine an optimal sample processing workflow that mitigates the WBC contamination. For example, through iterative testing, the analytics system may determine a set of protocols that minimize the WBC contamination, a set of clinical products that minimize the WBC contamination, a set of one or more sequencing devices that minimize the WBC contamination, or some combination thereof. The optimal sample processing workflow is applied to subsequent samples.

### VI. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

1.-18. (canceled)

19. A method for detecting white blood cell (WBC) contamination in a test sample comprising sequence reads corresponding to cell-free DNA (cfDNA) fragments, the method comprising:
determining, for each of a feature set of genomic loci as contamination markers, a coverage based on a count of the sequence reads of the cfDNA fragments that overlap a genomic locus;
determining, for each of the feature set of genomic loci, a statistical likelihood of observing the coverage at the genomic locus based on a distribution of coverage for the genomic locus generated from purified cfDNA samples;
generating a contamination metric for the test sample by combining the statistical likelihoods across the plurality of contamination markers;
determining whether the test sample has WBC contamination if the contamination metric for the test sample crosses a contamination threshold; and
in response to determining that the test sample has WBC contamination, performing one or more remedial measures.

20. The method of claim 19, further comprising identifying the contamination markers for detecting WBC contamination by:
obtaining a first set of cfDNA samples from a first cohort of subjects and a second set of WBC samples from the first cohort of subjects
determining, for each sample, a coverage of sequence reads overlapping each genomic locus in an initial set of genomic loci;
determining, for each genomic locus, a ratio of coverage between the WBC samples and the cfDNA samples; and
determining a feature set of genomic loci as contamination markers with ratios of coverage above a threshold.

21. The method of claim 19, further comprising training a contamination model for detecting white blood cell (WBC) contamination in a test sample comprising sequence reads corresponding to cell-free DNA (cfDNA) fragments, the method comprising:
obtaining a set of cfDNA samples from non-cancer subjects, each cfDNA sample comprising sequence reads corresponding to cfDNA fragments;
determining, for each sample, a coverage of sequence reads overlapping each genomic locus in a feature set of genomic loci as contamination markers;
generating, for each genomic locus in the feature set of genomic loci, a distribution of coverage based on coverage of a first subset of cfDNA samples;
determining, for each cfDNA sample of a second subset, a statistical likelihood of observing the coverage at each genomic locus based on the distribution of coverage for the genomic locus;
generating, for each cfDNA sample of the second subset, a contamination metric by combining the statistical likelihoods of the cfDNA sample across the genomic loci;
determining a distribution of contamination metric for the second subset of cfDNA samples; and
determining a contamination threshold based on the distribution of contamination metric,
wherein the contamination model comprises the distributions of coverage across the feature set of genomic loci, the distribution of contamination metric, and the contamination threshold.

22. (canceled)

23. The method of claim 19, wherein the purified cfDNA samples consist essentially of sequence reads corresponding to cfDNA fragments.

24. The method of claim 19, wherein the statistical likelihood of observing the coverage at each genomic locus is a z-score based on a mean and a standard deviation defining the distribution for the genomic locus.

25. The method of claim 24, wherein the contamination metric combines absolute values of the z-scores.

26. The method of claim 24, wherein determining whether the contamination metric for the test sample crosses the contamination threshold comprises determining whether the contamination metric is above the contamination threshold.

27. The method of claim 19, wherein the statistical likelihood of observing the coverage at each genomic locus is a p-value based on a mean and a standard deviation defining the distribution for the genomic locus.

28. The method of claim 27, wherein the contamination metric is a truncated p-value product of the p-values of the cfDNA sample across the genomic loci.

29. The method of claim 27, wherein determining whether the contamination metric for the test sample crosses the contamination threshold comprises determining whether the contamination metric is below the contamination threshold.

30. The method of claim 19, wherein performing the one or more remedial measures include any combination of:
providing a notification to a healthcare provider that the test sample has WBC contamination;
discarding the test sample;
labeling the test sample as contaminated;
providing a notification to a healthcare provider to collect a subsequent test sample;
providing a notification to a clinician of a likely source of WBC contamination; and
withholding the test sample from downstream analyses, optionally including cancer classification.

31.-46. (canceled)

47. A method for detecting white blood cell (WBC) contamination in a test sample comprising methylation sequence reads corresponding to cell-free DNA (cfDNA) fragments, the method comprising:
determining a methylation feature at each genomic locus in a feature set of genomic loci based on the methylation sequence reads;
generating a feature vector based on the methylation features over the feature set of genomic loci;
applying, to the feature vector of the test sample, a trained deconvolution model to predict tissue type fractions for the test sample;
generating a contamination metric for the test sample based on a distance of the tissue type fractions for the test sample relative to a distribution of tissue type fractions generated from cfDNA samples from non-cancer subjects, wherein the contamination metric indicates a likelihood that the cfDNA sample has WBC contamination;
determining whether the test sample has WBC contamination if the contamination metric for the test sample crosses a contamination threshold; and
in response to determining that the test sample has WBC contamination, performing one or more remedial measures.

48. The method of claim 47, further comprising training the deconvolution model configured to deconvolve tissue type fractional contribution by:
obtaining sets of cfDNA samples from different tissue types, wherein each cfDNA sample comprises methylation sequence reads corresponding to cfDNA fragments;
determining, for each cfDNA sample, a methylation feature at each genomic locus in an initial set of genomic loci based on the methylation sequence reads;
generating, for each sample, a feature vector based on the methylation features over the initial set of genomic loci; and
training the deconvolution model to predict tissue type fractions based on the feature vectors from the samples.

49. The method of claim 47, further comprising:
obtaining a set of cfDNA samples from non-cancer subjects, wherein each cfDNA sample comprises methylation sequence reads corresponding to cfDNA fragments;
determining, for each cfDNA sample, a methylation feature at each genomic locus in a feature set of genomic loci based on the methylation sequence reads;
generating, for each cfDNA sample, a feature vector based on the methylation features over the feature set of genomic loci;
applying, to the feature vector of each cfDNA sample, a trained deconvolution model to predict tissue type fractions for the cfDNA sample; and
building the distribution of the tissue type fractions for the cfDNA samples.

50. (canceled)

51. The method of claim 47, wherein the methylation feature at each genomic locus is one of:
methylation density across methylation sequence reads of the cfDNA sample at the genomic locus;
a count or a proportion of methylation sequence reads of the cfDNA sample that are highly methylated and overlap the genomic locus;
a count or a proportion of methylation sequence reads of the cfDNA sample that are highly unmethylated and overlap the genomic locus; and
a count or a proportion of methylation sequence reads having a particular methylation variant at the genomic locus.

52. The method of claim 47, wherein the distance is a Mahalanobis distance based on the distribution of tissue type fractions.

53. The method of claim 47, wherein the contamination metric is a p-value.

54.-70. (canceled)

71. A method for detecting white blood cell (WBC) contamination in a test sample comprising sequence reads corresponding to cell-free DNA (cfDNA) fragments, the method comprising:
determining a mean coverage of sequence reads overlapping a feature set of genomic loci;
determining a normalized coverage for each genomic locus in the feature set of genomic loci based on sequence reads overlapping the genomic locus normalized by the mean coverage of the sample;
applying a contamination model to determine a contamination metric as a fractional contribution of WBC-shed DNA to the test sample that maximizes a likelihood of observing the normalized coverages over the feature set based on distributions of coverage for cfDNA samples and for WBC samples for the feature set of genomic loci;
determining whether the test sample has WBC contamination if the contamination metric for the test sample crosses a contamination threshold; and
in response to determining that the test sample has WBC contamination, performing one or more remedial measures.

72. The method of claim 71, further comprising training the contamination model for detecting WBC contamination by:
obtaining a first set of cfDNA samples and a second set of WBC samples, each sample comprising sequence reads corresponding to DNA fragments;
determining, for each sample of the first set and the second set, a mean coverage of sequence reads overlapping an initial set of genomic loci;
determining, for each sample of the first set and the second set, a normalized coverage for each genomic locus in the initial set of genomic loci based on sequence reads overlapping the genomic locus normalized by the mean coverage of the sample; and
generating, for each genomic locus, a first distribution of coverage for cfDNA samples and a second distribution of coverage for WBC samples;
identifying highly discriminatory genomic loci between cfDNA samples and WBC samples based on the distributions of coverage;
determining a discriminatory score for each genomic locus with a two-sample t-test; and
determining a feature set of genomic loci as contamination markers based on the discriminatory scores.

73.-81. (canceled)

82. A method for training a cancer classifier with samples comprising sequence reads corresponding to cell-free DNA (cfDNA) fragments, the method comprising:
obtaining a first set of samples obtained from a first cohort of healthy subjects and a second set of samples obtained from a second cohort of subjects diagnosed with cancer;
applying a contamination model to the first set of samples and the second set of samples to determine a contamination metric for each sample indicating an amount of white blood cell (WBC) contamination in the sample;
determining one or more of the samples to be contaminated having a corresponding contamination metric above a contamination threshold;
filtering the contaminated samples, optionally wherein filtering comprises discard the contaminated samples;
determining a feature vector for each remaining sample based on the sequence reads of that sample; and
training the cancer classifier with the feature vectors for the remaining samples, wherein the trained cancer classifier is configured to predict likelihood of presence of cancer based on an input feature vector derived based on sequence reads in a test cfDNA sample.

83.-91. (canceled)
